(19) United States
(12) Patent Application Publication
(10) Pub. No.: US 2005/0272649 A1
(43) Pub. Date: Dec. 8, 2005

(54) Conjoint administration of morphogens and ACE inhibitors in treatment of chronic renal failure

(76) Inventors: Keith A. Hruska, St. Louis, MO (US); John McCartney, Holliston, MA (US); Marc F. Charette, Needham, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/650,326

(22) Filed: Aug. 28, 2003

Related U.S. Application Data
(60) Provisional application No. 60/406,431, filed on Aug. 28, 2002.

(51) Int. Cl. A61K 38/17; A61K 38/04; A61K 31/401

(52) U.S. Cl. 514/12; 514/423; 514/19

(57) ABSTRACT

The present invention provides reagents and methods for the treatment, and pharmaceuticals for use in the prevention and/or treatment, of chronic renal failure and other renal disorders in subjects (particularly mammalian subjects) renal replacement therapy. The methods involve the conjoint administration of ACE (Angiotensin-Converting Enzyme) inhibitors or Angiotensin II Receptor Antagonists (AIIRAs) with one or more OP/BMP family of proteins (morphogens, or inducers of morphogens, or agonists of the corresponding morphogen receptors, etc.). The invention also provides methods for implantation of renal cells induced with the conjoint administration of ACE inhibitors or AIIRAs with those morphogens.

### RELATED APPLICATIONS

This application claims priority to the filing date of U.S. Provisional Application No. 60/406,431, filed Aug. 28, 2002, entitled “Conjoint Administration of Morphogens and ACE Inhibitors In Treatment of Chronic Renal Failure,” the entire teachings of which are hereby incorporated by reference.

### GOVERNMENT FUNDING

A portion of this invention was made with U.S. government support under grant number DK59602, DK09976 from the National Institutes of Health (NIH), and under grant number DK20579 from the NIH supported Diabetes Research and Training Center (DRTC) of Washington University. The government may have certain rights in this invention.

### BACKGROUND OF THE INVENTION

The mammalian renal system serves primary roles both in the removal of catabolic waste products from the bloodstream and in the maintenance of fluid and electrolyte balances in the body. Renal failures are, therefore, life-threatening conditions in which the build-up of catabolites and other toxins, and/or the development of significant imbalances in electrolytes or fluids, may lead to the failure of other major organs systems and death. As a general matter, renal failure is classified as “acute” or “chronic.” As detailed below, the differences between these two conditions are not merely a matter of severity or rapidity but, rather, reflect differences in etiology, prognosis, and treatment.

Acute Renal Failure:

Acute renal failure is defined as an abrupt cessation or substantial reduction of renal function and, in as many as 90-95% of cases, may be secondary to trauma, surgery or another acute medical condition. Acute renal failure may be due to pre-renal causes (e.g., decreased cardiac output, hypovolemia, altered vascular resistance) or to post-renal causes (e.g., obstructions or constrictions of the ureters, bladder or urethra) which do not directly involve the kidneys and which, if treated quickly, will not entail significant loss of nephrons or other damage to the kidneys. Alternatively, acute renal failure may be due to intrinsic renal causes which involve a more direct insult or injury to the kidneys, and which may entail permanent damage to-the nephrons or other kidney structures. Intrinsic causes of acute renal failure include but are not limited to infectious diseases (e.g., various bacterial, viral or parasitic infections), inflammatory diseases (e.g., glomerulonephritis, systemic lupus erythematosus), ischemia (e.g., renal artery occlusion), toxic syndromes (e.g., heavy metal poisoning, side-effects of antimicrobial treatments or chemotherapy), and direct traumas.

The diagnosis and treatment of acute renal failure is as varied as its causes. In human patients, oliguria (urine output<400 ml/day) or anuria (urine output<50 ml/day) may be present in 50-70% of cases, BUN levels may climb 10-20 mg/dL/day or faster, plasma creatinine levels may climb 0.5-1.0 mg/dL/day, and metabolic acidosis is almost always present. If not treated, the electrolyte and fluid imbalances (e.g., hyperkalemia, acidosis, edema) associated with acute renal failure may lead to life-threatening arrhythmia, congestive heart failure, or multiple organ system failures. Present therapies are typically directed at the underlying causes of the acute renal failure (e.g., pre-renal, post-renal, or infectious causes) and management of the complications. Due to the severity of acute renal failure, episodes rarely last longer than several weeks without mortality and are treated on an in-patient basis.

Chronic Renal Failure:

Chronic renal failure (CRF) is the progressive loss of kidney function. The kidneys attempt to compensate for renal damage by hyperfiltration (excessive straining of the blood) within the remaining functional nephrons (filtering units that consist of a glomerulus and corresponding tubule). Over time, hyperfiltration causes further loss of function.

Chronic loss of function causes generalized wasting (shrinking in size) and progressive scarring within all parts of the kidneys. In time, overall scarring obscures the site of the initial damage. Yet, it is not until over 70% of the normal combined function of both kidneys is lost that most patients begin to experience symptoms of kidney failure. Thus, chronic renal failure may be defined as a progressive, permanent and significant reduction of the glomerular filtration rate (GFR) due to a significant and continuing loss of nephrons.

Chronic renal failure typically begins from a point at which a chronic renal insufficiency (i.e., a permanent decrease in renal function of at least 50-60%) has resulted from some insult to the renal tissues which has caused a significant loss of nephron units. The initial insult may or may not have been associated with an episode of acute renal failure. Irrespective of the nature of the initial insult, chronic renal failure manifests a “final common path” of signs and symptoms as nephrons are progressively lost and GFR progressively declines. This progressive deterioration in renal function is slow, typically spanning many years or decades in human patients, but seemingly inevitable.

The early stage of chronic renal failure typically begins when GFR has been reduced to approximately one-third of normal (e.g., 30-40 ml/min for an average human adult). As a result of the significant nephron loss, and in an apparent “attempt” to maintain the overall GFR with fewer nephrons, the average single nephron GFR (SNGFR) is increased by adaptations of the remaining nephrons. at both the structural and functional level. One structural manifestation of this adaptation, readily detectable by microscopic examination of biopsy samples, is a “compensatory hypertrophy” of both the glomeruli and the tubules of the kidney, a process which literally increases the volume of filtrate which can be produced by each remaining nephron by literal enlargement of the glomeruli and tubules. Indeed, as a result of the hypertrophy or dilation of the collecting ducts, the urine of subjects with chronic renal failure often contains broad “casts,” typically 2-6 times normal diameter, which aid in diagnosis and have also been referred to as “renal failure casts.” At the same time, there are functional changes in the remaining nephrons, such as decreased absorption or increased secretion of normally excreted solutes, which may be responses to hormonal or paracrine changes elsewhere in the body (e.g., increasing levels of parathyroid hormone (PTH) in response to changes in serum levels of calcium and phosphate).

These adaptations in early stage chronic renal failure are not successful in completely restoring GFR or other parameters of renal function and, in fact, subject the remaining nephrons to increased risk of loss. For example, the increased SNGFR is associated with mechanical stresses on the glomerulus due to hyper-tension and hyper-perfusion. The loss of integrity of podocyte junctures leads to increased permeability of the glomerulus to macromolecules or “leakiness” of the glomerular capsule. Proliferative effects are also observed in mesangial, epithelial and endothelial cells, as well as increases in the deposition of collagen and other matrix proteins. Sclerosis of both the glomeruli and tubules is another common symptom of the hypertrophied nephrons and the risk of coagulation in the glomerulus is increased. In particular, these adaptations of the remaining nephrons, by pushing the SNGFR well beyond its normal level, actually decrease the capacity of the remaining nephrons to respond to acute changes in water, solute, or acid loads and, therefore, actually increase the probability of additional nephron loss.

As chronic renal failure progresses, and GFR continues to decline to less than 10% of normal (e.g., 5-10 mL/min), the subject enters end-stage renal disease (ESRD). During this phase, the inability of the remaining nephrons to adequately remove waste products from the blood, while retaining useful products and maintaining fluid and electrolyte balance, leads to a rapid decline in which many organ systems, and particularly the cardiovascular system, may begin to fail. For example, BUN and creatinine levels may be expected to rise and, at BUN levels of 60-100 mg/dL and serum creatinine levels of 8-12 mg/dL, a uremic syndrome will typically develop in which the kidneys can no longer remove the end products of nitrogen metabolism. At this point, renal failure will rapidly progress to death unless the subject receives renal replacement therapy (i.e., chronic hemodialysis, continuous peritoneal dialysis, or kidney transplantation).

Approximately 600 patients per million receive chronic dialysis each year in the United States, at an average cost approaching $60,000-$80,000 per patient per year. Of the new cases of end-stage renal disease each year, approximately 28-33% are due to diabetic nephropathy (or diabetic glomerulopathy or diabetic renal hypertrophy), 24-29% are due to hypertensive nephrosclerosis (or hypertensive glomerulosclerosis), and 15-22% are due to glomerulonephritis.

The 5-year survival rate for all chronic dialysis patients is approximately 40%, but for patients over 65, the rate drops to approximately 20%.

Morphogens and Growth Factors:

A great many proteins have now been identified which appear to act as morphogenetic or growth factors, regulating cell proliferation or differentiation. Typically these growth factors exert their effects on specific sets or subsets of cells or tissues. Thus, for example, epidermal growth factors, nerve growth factors, fibroblast growth factors, various hormones, and many other proteins inducing or inhibiting cell proliferation or differentiation have been identified and shown to affect some subgroup of cells or tissues.

One group of morphogenetic proteins, referred to herein as “morphogens,” includes members of the family of osteogenic proteins/bone morphogenetic proteins (OP/BMPs) which were initially identified by their ability to induce ectopic, endochondral bone morphogenesis.

Subsequent characterization of the nucleic acid and amino acid sequences of the BMPs has shown them to be a subgroup of the TGF-β superfamily of growth factors. Members of this morphogen family have now been shown to include the mammalian osteogenic protein-1 (OP-1, also known as BMP-7), osteogenic protein-2 (OP-2), osteogenic protein-3 (OP-3), BMP-2 (also known as BMP-2A or CBMP-2A), BMP-3, BMP-4 (also known as BMP-2B or CBMP-2B), BMP-5, BMP-6, Vgr-1, and GDF-1, as well as the Xenopus homologue Vgl and the Drosophila homologues DPP and 60A. Members of this family encode secreted polypeptides, that share common structural features and that are similarly processed from pro-proteins to yield carboxy terminal mature proteins having a conserved pattern of cysteines. The active forms of these proteins are either disulfide-bonded homodimers of a single family member, or heterodimers of two different members (see, e.g., Massague (1990) Annu. Rev. Cell Biol. 6:597; Sampath, et al., (1990) J. Biol. Chem. 265:13198).

The members of the morphogen family of proteins are expressed in a variety of tissues during development. BMP-3 for, example, has been shown to be expressed in developing human lung and kidney (Vukicevic et al. (1994) J. Histochem. Cytochem. 42:869-875), BMP-4 has been-shown to be expressed in the developing limbs, heart, facial processes and condensed mesenchyme associated with early whisker follicles in embryonic mice (Jones, et al. (1991) Development 111: 531-542), and OP-1 (BMP-7) has been shown immunohistochemically to be associated with basement membranes in human embryos, including those of the developing lungs, pancreas, skin, and convoluted tubules of kidneys (Vukicevic, et al. (1994) Biochem. Biophys. Res. Commun. 198: 693-700). Some of the morphogens (e.g., OP-2 and BMP-2) were not detected in analyses of adult tissues, suggesting only an early developmental role for these morphogens (Ozkaynak, et. al. (1992) J. Biol. Chem. 267: 25220-25227). In contrast, high levels of murine OP-1 expression have been observed in adult mouse kidneys (Ozkaynak, et al. (1991) Biochem. Biophys. Res. Commun. 179: 116-123). This suggests a possible role for OP-1 synthesized in the kidney as a paracrine regulator of bone growth, and would be consistent with the role of the kidneys in both calcium regulation and bone homeostasis.

A great variety of growth factors have been considered which may participate in the regulation of the growth and repair of renal tissues (reviewed in, e.g., Toback (1992) Kidney Intl. 41: 226-246). For example, EGF, TGF-α, TGF-β, IGF-I, IGMI, PDGF, FGF, Renin/Angiotensin II, IL-1 and OP-1 have all been found to be expressed by various adult renal cells or tissues and to have effects on renal cell proliferation or differentiation (see, Toback (1992) supra, Ozkaynak, et al. (1991) supra). In addition, several of these have been found to be expressed in the developing kidney, including IGF-I, TGF-β and OP-1 (reviewed in, e.g., Bard, et al. (1994) Mech. Development 48: 3-11).

Interestingly, TGF-β has been shown in a murine metanephric organ culture system to retard overall growth and segmental differentiation of all segments of developing nephrons except the thick ascending limb-early distal tubules (Avner and Sweeney (1990) Pediatr. Nephrol. 4: 372-377). In addition, TGF-β expression has been found to be increased in several models of renal disease, suggesting that TGF-β mediated increases in the synthesis of extracellular matrix components may be involved in the etiology of diabetic nephropathy (or diabetic glomerulopathy or diabetic renal hypertrophy), renal fibrosis, glomerulosclerosis and glomerulonephritis, interstitial fibrosis, and hypertensive nephrosclerosis (Shankland, et al. (1994) Kidney Intl. 46: 430-442; Yamamoto, et al. (1994) Kidney Intl. 45:916-927; Yamamoto, et al. (1993) PNAS 90: 1814 Tamaki, et al. (1994) Kidney Intl. 45:525-536; Border, et al. (1990) Nature 346: 371-374; Hamaguchi, et al. (1995) Hypertension 26: 199-207).

Also of interest is the fact that serum levels of human growth hormone (GH) are elevated in subjects with chronic renal failure (Wright et al. (1968) Lancet 2: 798; Samaan and Freeman (1970) Metabolism 19: 102). Recombinant GH has been shown to help maintain protein balance in malnourished chronic renal failure patients, and to promote “catch-up” growth in children with chronic renal failure. It has been suggested that these effects are mediated by IGF-I (see, e.g., Kopple (1992) Miner. Electrolyte Metab. 18: 269-275). Although some studies have found that the administration of IGF-I increases renal plasma flow and GFR in chronic renal failure patients (e.g., Guler, et al. (1989) PNAS 86:2868-2872; Hirschberg, et al. (1993) Kidney Intl. 43:387-397), other studies have found that this effect is merely transient (Miller, et al. (1994) Kidney Intl. 46: 201-207).

Thus, although some growth factors have been shown to be expressed in both developing and adult renal tissues, and although at least one has been shown to increase renal function in the short term, none has yet been shown to be of therapeutic benefit in preventing, inhibiting, or delaying the progressive loss of renal function that characterizes chronic renal failure. A need remains, therefore, for treatments which will prevent the progressive loss of renal function which causes hundreds of thousand of patients to become dependent upon chronic dialysis, and which results in the premature deaths of tens of thousands each year.

### SUMMARY OF THE INVENTION

The present invention is directed to methods of treatment, and pharmaceutical preparations for use in the treatment, of vertebrate subjects (preferably mammalian subjects) in, or at risk of, chronic renal failure, or at risk of the need for renal replacement therapy by using a combination of an Angiotensin-Converting Enzyme inhibitor (ACEI) and a morphogen. Suitable subjects include subjects already afflicted with chronic renal failure, or which have already received renal replacement therapy, as well as any subject reasonably expected to suffer a progressive loss of renal function associated with progressive loss of functioning nephron units. Whether a particular subject is at risk is a determination which may routinely be made by one of ordinary skill in the relevant medical or veterinary art. Subjects in, or at risk of, chronic renal failure, or at risk of the need for renal replacement therapy, include but are not limited to the following: subjects which may be regarded as afflicted with chronic renal failure, end-stage renal disease, chronic diabetic nephropathy, hypertensive nephrosclerosis, chronic glomerulonephritis, hereditary nephritis, and/or renal dysplasia; subjects having a biopsy indicating glomerular hypertrophy, tubular hypertrophy, chronic glomerulosclerosis, and/or chronic tubulointerstitial sclerosis; subjects having an-ultrasound, MRI, CAT scan, or other non-invasive examination indicating renal fibrosis; subject shaving an unusual number of broad casts present in urinary sediment; subjects having a GFR which is chronically less than about 50%, and more particularly less than about 40%, 30% or 20%, of the expected GFR for the subject; human male subjects weighing at least about 50 kg and having a GFR which is chronically less than about 50 ml/min, and more particularly less than about 40 ml/min, 30 ml/min or 20 ml/min; human female subjects weighing at least about 40 kg and having a GFR which is chronically less than about 40 mL/min, and more particularly less than about 30 mL/min, 20 ml/min or 10 ml/min; subjects possessing a number of functional nephron units which is less than about 50%, and more particularly less than about 40%, 30% or 20%, of the number of functional nephron units possessed by a healthy but otherwise similar subject; subjects which have a single kidney; and subjects which are kidney transplant recipients.

The methods and compositions of this invention capitalize in part upon the discovery that certain morphogens of eukaryotic origin and inhibitors of ACE (Angiotensin-Converting Enzyme) may be used conjointly as therapeutic agents in the treatment of subjects at risk, as defined herein, of chronic renal failure or the need for renal replacement therapy. Generally, these proteins are members of the osteogenic protein/bone morphogenetic protein (OP/BMP) family of proteins or analogs thereof. In a preferred embodiment, the ACE inhibitor is enalapril.

Thus, useful OP/BMP morphogens of the invention include polypeptides, or functional variants of polypeptides, comprising at least the C-terminal six- or seven-cysteine domain of a mammalian protein selected from OP-1, OP-2, OP-3, BMP2, BMP3, BMP4, BMP5, BMP6, BMP9, and proteins which exhibit at least 70% or, more preferably, 75% or 80%, 85%, 90%, 95%, 99% amino acid sequence homology, or at least 50% identity, more preferably 55%, 60%, 65%, 70%, 80%, 90%, 99% or more identity, with the amino acid sequence of the seven-cysteine domain of any of the morphogens described above, such as human OP-1; and which are (a) capable of inducing chondrogenesis in the Reddi-Sampath ectopic bone assay (Sampath and Reddi (1981), Proc. Natl. Acad. Sci. USA 78: 7599-7603) or a substantially equivalent assay, (b) capable of significantly preventing, inhibiting, delaying or alleviating the progressive loss of renal function in a standard animal model of chronicrenal failure, or (c) capable of causing a clinically significant improvement in a standard marker of renal function when administered to a mammal in, or at risk of, chronic renal failure. More generally speaking, the invention provides for the use of “morphogens” which are dimeric proteins that induce morphogenesis of one or more eukaryotic (e.g., mammalian) cells, tissues or organs.

Of particular interest herein are morphogens that induce morphogenesis at least of mammalian renal tissue, including formation of functional renal epithelium and, in particular, functional glomerular and tubular epithelium. Morphogens comprise a pair of polypeptides that, when folded, adopt a configuration suitable for the resulting dimeric protein to elicit morphogenetic responses in cells and tissues displaying receptors specific for said morphogen. That is, morphogens generally induce all of the following biological functions in a morphogenically permissive environment: stimulating proliferation of progenitor cells; stimulating the differentiation of progenitor cells; stimulating the proliferation of differentiated cells; and supporting the growth and maintenance of differentiated cells. “Progenitor” cells are uncommitted cells that are competent to differentiate into one or more specific types of differentiated cells, depending on their genomic repertoire and the tissue specificity of the permissive environment in which morphogenesis is induced. Morphogens further can delay or mitigate the onset of senescence- or quiescence-associated loss of phenotype and/or tissue function. Morphogens still further can stimulate phenotypic expression of differentiated cells, including expression of metabolic and/or functional, e.g., secretory, properties thereof. In addition, morphogens can induce redifferentiation of committed cells under appropriate environmental conditions. As noted above, morphogens that induce proliferation and/or differentiation at least of mammalian renal tissue, and/or support the growth, maintenance and/or functional properties of mammalian nephrons, are of particular interest herein.

In preferred embodiments, the pair of morphogen polypeptides have amino acid sequence search comprising a sequence that shares a defined relationship with an amino acid sequence of a reference morphogen. Herein, preferred morphogen polypeptides share a defined relationship with a sequence present in morphogenically active human OP-1. However, anyone or more of the naturally occurring or biosynthetic sequences disclosed herein similarly could be used as a reference sequence. Preferred morphogen polypeptides share a defined relationship with at least the C-terminal six cysteine domain of human OP-1, residues 43-139 of SEQ ID NO: 1. Preferably, morphogen polypeptides share a defined relationship with at least the C-terminal seven cysteine domain of human OP-1, residues 38-139 of SEQ ID NO: 1. That is, preferred morphogen polypeptides in a dimeric protein with morphogenic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric morphogen protein, including their ability to form such intra- or inter-chain disulfide bonds as may be necessary for morphogenic activity.

Functionally equivalent sequences further include those wherein one or more amino acid residues differ from the corresponding residues of a reference morphogen sequence, e.g., the C-terminal seven cysteine domain (or “skeleton”) of human OP-1, provided that this difference does not destroy morphogenic activity. Accordingly, conservative substitutions of corresponding amino acids in the reference sequence are preferred. Amino acid residues that are “conservative substitutions” for corresponding residues in a reference sequence are those that are physically or functionally similar to the corresponding reference residues, e.g., that have similar size, shape, electric charge, chemical properties including the ability to form covalent or hydrogen bonds, or the like. Particularly preferred conservative substitutions are those fulfilling the criteria defined for an “accepted point mutation” in Dayhoff et al. (1978), 5 Atlas of Protein Sequence and Structure, Suppl. 3, ch. 22 (pp. 354-352), Nad. Biomed. Res. Found., Washington, D.C. (see below), the teachings of which are incorporated by reference herein.

In certain embodiments, a polypeptide suspected of being functionally equivalent to a reference morphogen polypeptide is aligned therewith using the method of Needleman, et al. (1970), J. Mol. Biol. 48: 443-453, implemented conveniently by computer programs such as the Align program or other improved successors/variants (DNAstar, Inc.). For example, the MegAlign program of the Lasergene 5.0 (DNAStar, Inc.) offers several multi-sequence alignment methods (J. Hein method, see Hein, J. J. (1990). “Unified approach to alignment and phylogenies.” In Methods in Enzymology, Vol. 183: pp. 626-645; Clustal V method, see Higgins, D. G. and P. M. Sharp (1989). “Fast and sensitive multiple sequence alignments on a microcomputer.” CABIOS, Vol. 5, No. 2: pp. 151-153; Clustal W method, see J. D. Thompson, et al. (1994). Nucleic Acids Research, Vol 22, pp. 4673-80), each with different algorithms, and each offers user opportunities to define parameters such as gaps. Specifically, gaps and insertions are arranged to achieve the highest degree of correlation between the amino acids of the two sequences being compared, with user specified penalties—the so-called Gap Penalty (the amount deducted from the alignment score for each gap in the alignment. Gaps of different sizes carry the same penalty), and the Gap Length Penalty (the value deducted from the alignment score after first multiplying it by the length of gaps. Longer gaps have a greater penalty than shorter gaps). The aligned sequences will then be used to calculate a percent identity (homology) between the candidate and reference sequences. Sequence homology between a section of the aligned sequences can also be generated. In a preferred embodiment, each amino acid of a gap or insertion counts as a mismatch for measuring % identity purpose.

Of particular interest herein are morphogens, which, when provided to the kidney of a mammal, induce or maintain the normal state of differentiation and growth of nephron units. Of still more particular interest herein are morphogens which, when administered to a mammal, prevent, inhibit or delay the development of compensatory hypertrophy, including glomerular hypertrophy and/or tubular hypertrophy. Such morphogens can be used to treat a mammal in, or at risk of, chronic renal failure by preventing, inhibiting or delaying the progressive loss of functional nephron units and the consequent progressive loss of renal function.

The present invention alternatively can be practiced with methods and compositions comprising a morphogen stimulating agent or morphogen inducer in lieu of a morphogen. A “morphogen inducer” is a compound that stimulates in vivo production, e.g., expression, of a therapeutically effective concentration of an endogenous morphogen in the body of a mammal sufficient to regenerate or maintain renal tissue and/or to inhibit additional loss thereof. Such compounds are understood to include substances which, when administered to a mammal, act on cells of tissue(s) or organ(s) that normally are competent to produce and/or secrete a morphogen encoded within the genome of the mammal, and which cause the endogenous level of the morphogen in the mammal's body to be altered. Endogenous or administered morphogens can act as endocrine, paracrine or autocrine factors. That is, endogenous morphogens can be synthesized by the cells in which morphogenetic responses are induced, by neighboring cells, or by cells of a distant tissue, in which circumstances the secreted endogenous morphogen is transported to the site of morphogenesis, e.g., by the individual's bloodstream. In preferred embodiments, the agent stimulates expression and/or secretion of an endogenous morphogen so as to increase amounts thereof in renal tissues.

In still other embodiments, an agent which acts as an agonist of a morphogen receptor may be administered instead of the morphogen itself. An “agonist” of a receptor means a compound which binds to the receptor and for which such binding has a similar functional result as binding of the natural, endogenous ligand of the receptor. That is, the compound must, upon interaction with the receptor, produce the same or substantially similar transmembrane and/or intracellular effects as the endogenous ligand. Thus, an agonist of a morphogen receptor binds to the receptor and such binding has the same or a similar functional result as morphogen binding (e.g., induction of morphogenesis). The activity or potency of an agonist can be less than that of the natural ligand, in which case the agonist is said to be a “partial agonist,” or it can be equal to or greater than that of the natural ligand, in which case it is said to be a “full agonist.” Thus, for example, a small peptide or other molecule which can mimic the activity of a morphogen in binding to and activating the morphogen's receptor may be employed as an equivalent of the morphogen. Preferably the agonist is a full agonist, but partial morphogen receptor agonists may also be advantageously employed. Methods of identifying such agonists are known in the art and include assays for compounds which induce morphogen-mediated responses (e.g., induction of differentiation of metanephric mesenchyme, induction of endochondral bone formation, and the like). Such an agent may also be referred to as a morphogen “mimic,” “mimetic,” or “analog.”

The OP/BMP morphogens, or the morphogen inducers/agonists of morphogen receptors, or ACEI of the invention, may be administered by any route of administration which is compatible with the selected agent, and may be formulated with any pharmaceutically acceptable carrier appropriate to the route of administration. Preferred routes of administration are parenteral and, in particular, intravenous, intraperitoneal, and renal intracapsular. Treatments are also preferably conducted over an extended period on an out-patient basis. Daily dosages of the morphogens are expected to be in the range of about 0.01-1000 μg/kg body weight, and more preferably about 10-300 μg/kg body weight, although precise dosages will vary depending upon the particular therapeutic agent employed and the particular subject's medical condition and history.

Finally, in yet further embodiments, renal cells may be implanted into the kidney of a subject in, or at risk of, chronic renal failure, or at risk of needing renal replacement therapy, in order to serve as a source of morphogen and/or to provide a source of additional functional renal tissue. Preferably, the cells are induced to undergo metanephric differentiation by treatment with a morphogen (e.g., OP-1) either before or after implantation. These cells may be renal mesenchymal progenitor cells, or renal mesenchymal progenitor cells which have been induced to undergo metanephric differentiation. The cells may be derived from a donor (e.g., a tissue-type matched donor, sibling, identical twin), or may be derived from a tissue culture (e.g., undifferentiated renal mesenchyme culture, fetal renal tissue culture), or may be explanted from the subject and then be re-implanted after proliferation and/or differentiation.

The methods of the present invention are useful in preventing, inhibiting or delaying the progressive loss of functional nephron units, and the consequent progressive loss of renal function, which typify chronic renal failure. As such they are of great value in preventing or delaying the need for chronic dialysis or renal replacement therapy in subjects with chronic renal insufficiency, or reducing the necessary frequency of chronic renal dialysis in subjects with end-stage renal disease. As such, they are useful in prolonging the lives, and in maintaining the quality of life, of subjects at risk of, or already afflicted with, chronic renal failure.

In a related aspect, the invention also contemplates conjoint administration of Angiotensin II Receptor Antagonists/Blockers (AIIRAs) with certain protein-based morphogens to subjects in, or at risk of, chronic renal failure, in order to reduce mortality and/or morbidity rates, and to prevent, inhibit, delay or alleviate the progressive loss of renal function which characterizes chronic renal failure. Alternatively, or in addition, conjoint administration of angiotensin II receptor blockers with the morphogens of the present invention can prevent, inhibit or delay the progressive loss of functional nephron units and the progressive decline in glomerular filtration rate (GFR) which slowly but inevitably leads to the need for renal replacement therapy (i.e., renal transplant or chronic dialysis) or death. In preferred embodiments, the therapeutic agents of the invention are members of the osteogenic protein/bone morphogenetic protein (OP/BMP) family within the TGF-superfamily of proteins, and angiotensin II receptor blockers.

### BRIEF DESCRIPTION OF THE FIGURES

FIG. 1 The long term streptozotocin induced model of diabetic nephropathy. DM was induced at week 0 and the rats maintained as described in methods. (A) At 16 weeks, kidney weights had increased 1.8 fold in DM compared to normal (1.42±0.02 versus 0.81±0.02 g, p<0.01). (B) GFR also increased 3.2 fold compare to normal (1.56±0.27 versus 0.49±0.04 ml/mim/100 g body wt, p<0.01). After 16 weeks of vehicle treatment, kidney weights had not changed significantly (A), but the GFR was decreased 75% to even lower than normal at 32 weeks (0.34±0.02 versus 0.55±0.02) (B).

FIG. 2 Effects of BMP-7 and enalapril treatments on DM induced renal hypertrophy. DM was induced at week 0. Treatment of BMP-7, or enalapril, or vehicle was began at week 16 and finished at week 32. At 16 weeks, kidney weight increased to 1.42 in DM compared to normal. After 16 weeks of vehicle treatment, the kidney weight was still elevated (1.44±0.04 g). The kidney weights of the BMP-7 and enalapril treated groups were significantly decreased to 1.10±0.03 in BMP-7 high dose group and 1.09±0.04 in enalapril treated group, p<0.01 compared to DM vehicle treated.

FIG. 3 Effects of BMP-7 and enalapril treatments on GFR. In DM rats the GFR was increased 3.2-fold compare to normal at 16 weeks (1.56±0.27 versus 0.49±0.04 ml/mim/100 g body wt, p<0.01), but by 32 weeks the GFR was decreased to lower than normal during vehicle treatment (0.34±0.02 versus 0.55±0.02). The BMP-7 and enalapril treatments restored GFR to normal or slightly above normal. The GFR of the BMP-7 high dose animals were significantly greater than the GFR of DM group (vehicle treated) (0.70±0.08 versus 0.34±0.02, p<0.05). There was a dose-dependent organization of the GFR in the BMP-7 treated groups (0.59±0.07, 0.62±0.09, and 0.70±0.08, respectively).

FIG. 4 Effects of BMP-7 and enalapril treatments on urine protein excretion in DM. DM was induced at week 0. Treatment of BMP-7, or Enalapril, or vehicle was began at week 16 and finished at week 32. Diabetic rats exhibited a pronounced increase in albumin excretion rate compared with nondiabetic rats at both 16 weeks (35.63±13.35 versus 3.76±0.39 mg/day) and 32 weeks (174.4±52.50 versus 8.24±1.28, p<0.01). This response was markedly reduced by BMP-7 and enalapril treatment (p<0.01, DM versus BMP10; p<0.001, DM versus BMP30, BMP100 and Enalapril). There was a dose-dependent inverse order in the levels of urinary protein in the BMP-7 treated groups from low to high dose (59.46±21.84, 33.02±9.11, and 14.27±3.50, respectively). The Enalapril treatment group had urinary protein excretion levels similar to the intermediate dose BMP-7 group.

FIG. 5 Coronal sections of kidneys stained with periodic acid Schiff to highlight basement membranes and mesangial matrix. Panel A is a section of a kidney from a 16 week normal animal. B is a section of a kidney from a 16 week diabetic animal. Glomerular hypertrophy and early increases in mesangial matrix were present. There was evidence of glomerular (arrowhead) and tubular (arrow) basement membrane thickening. C is a section of a kidney from a 32 week diabetic vehicle-treated animal. One glomerulus is sclerotic and both are hypertrophied. There are segments of collapsed glomerular tuft with sparseness of normal cellular elements. D is a section of a kidney from an animal treated with BMP-7 30 μg/kg body wt IV twice a week. E is a section of a kidney from an animal treated with BMP-7 100 μg/kg body wt IV twice a week. F is a section of a kidney from an animal treated with Enalapril 100 mg/l in drinking water. All of the three BMP-7 dosages and enalapril treatment decreased glomerular sclerosis, but the enalapril treatment had more mesangial matrix accumulation.

FIG. 6 Coronal sections of kidneys stained with Gomori's Trichrome for collagen. With this stain, collagen fibrils stain blue, whereas the cells stain red. A and B are sections from kidneys of two normal control animals maintained in the animal facility with the diabetic animals for 32 weeks. C is a section from a diabetic vehicle-treated animal.

Arrow shows early interstitial matrix accumulation. The glomeruli are hypertrophic and one has segmental sclerosis. D, E, and F are sections of kidneys from animals treated with BMP-7 10, 30, and 100 μg/kg body wt. IV twice a week, respectively. Kidneys from the 10 μg/kg body wt. dose animals had greater mesangial matrix accumulation than the two higher doses which were very similar to normal except for residual glomerular hypertrophy.

FIG. 7 Effects of BMP-7 and enalapril treatments on glomerular and interstitial areas in DM rats. (A) Glomerular area. Diabetic rats had a significantly larger glomerular area than normal control rats (1.28±0.03 versus 0.90±0.02×104 μm², p<0.001). All the treatment groups partially reversed the glomerular hypertrophy (p<0.001). (B) Interstitial area. The cortical interstitial area was increased from 9.0±0.6% sections in nondiabetic rat kidneys to 13.1±0.7% in the DM rats. The BMP-7 high dose and enalapril treatment significantly decreased interstitial area (10.7±0.3%, p<0.05; 10.3±0.4%, p<0.01, respectively) when compared to vehicle treated DM.

FIG. 8 Effects of BMP-7 and enalapril treatments on glomerulosclerosis. Diabetic rats exhibited a significant increase in prevalence of sclerotic glomeruli compared with nondiabetic rats at 32 weeks (10.7±4.0% versus 0.7±0.2%, p<0.001). This response was markedly reduced by BMP-7 and enalapril treatment. There was a dose-dependent ordering in the BMP-7 treated groups (4.1±1.4%, p<0.05; 3.5±0.8%, p<0.01; and 2.1±0.3%, p<0.001, respectively). The effect of high dose BMP-7 was significantly better than that of Enalapril.

FIG. 9 Effects of Enalapril and BMP-7 therapies on systolic blood pressure of diabetic rats. Blood pressures were obtained by the artery cuff method. By 16 weeks the diabetic animals were significantly hypertensive. The hypertension was stable in vehicle treated rats until week 28 when systolic blood pressures began to increase again. Over two months Enalapril therapy restored blood pressure to normal. BMP-7 therapy did not affect blood pressure until the last four weeks of therapy.

FIG. 10 Loss of BMP-7 renal expression in diabetes and restoration with therapy. (A) The 32-week vehicle treated diabetic kidneys had complete loss of BMP-7 message. Both BMP-7 and Enalapril therapy restored the normal distribution of BMP-7 expression. The levels of BMP-7 message in the BMP-7 and Enalapril treated kidneys appeared to be higher than the normal animals. (B) Glomerular expression of BMP-7 during BMP-7 therapy. Bright and dark field sections demonstrate significant BMP-7 expression in a glomerulus of a BMP-7 treated animal. These results were consistent in three separate experiments with different kidneys.

FIG. 11 Induction of Wnt 4 expression by diabetes and effects of Enalapril and BMP-7 therapy. Wnt4 was not significantly expressed in normal kidneys. However, there was generalized renal expression of Wnt4 in vehicle treated diabetic rats. The expression was both in tubular epithelial cells and the glomeruli. BMP-7 therapy and Enalapril therapy had no effect on Wnt4 expression. The results were consistent in three separate experiments involving different kidneys.

FIG. 12 Schematic diagram of the five-sixths nephrectomy (5/6 NPX) Chronic Renal Failure (CRF) injury model.

FIG. 13 OP-1 did not dramatically lower the blood pressure level in the 5/6 nephrectomy model of Chronic Renal Failure.

FIG. 14 OP-1 significantly reduced the proteinuria level in the 5/6 nephrectomy model of Chronic Renal Failure.

FIG. 15 In animals conjointly administered with morphogen (OP-1) and ACE inhibitor (enalapril), there is no additional benefit in reducing the blood pressure of nephrectomized animals to normal level as compared to animals treated by the ACE inhibitor (enalapril) alone.

FIG. 16 Conjoint administration of morphogen (OP-1) and ACEI (enalapril) is more effective in reducing the proteinuria level in nephrectomized animals than ACEI treatment alone.

FIG. 17 Schematic diagram of the Unilateral Ureteral Obstruction (UUO) Renal Fibrosis Model.

FIG. 18 Morphogen OP-1/BMP-7 Inhibits renal fibrosis in the Unilateral Ureteral Obstruction model.

FIG. 19 The mechanism of morphogen (OP-1)-induced renal protection is associated with prevention of tubular atrophy, an effect not shared with ACEI enalapril.

FIG. 20 Both morphogen and ACEI improves renal function as measured by GFR. However, morphogen OP-1 is more efficacious than ACEI enalapril in improving the glomerular filtration rate as evidenced by the inulin clearance rate in the Unilateral Ureteral Obstruction model.

FIG. 21 Morphogen (OP-1), but not ACE inhibitor, significantly reduced the loss of medullary tissue in the kidney in the Unilateral Ureteral Obstruction model.

FIG. 22 Schematic diagram of Streptozotocin-induced rat model of diabetic nephropathy.

FIG. 23 Morphogen (OP-1), but not ACEI enalapril, significantly increased the glomerular filtration rate (GFR) in the Streptozotocin-induced rat model of diabetic nephropathy.

FIG. 24 Administration of morphogen (OP-1) alone, or ACEI enalapril alone, or conjoint administration of morphogen and ACEI significantly reduced the proteinuria level in the Streptozotocin-induced rat model of diabetic nephropathy.

FIG. 25 Schematic diagram of the Alloxan-induced rat model of diabetic nephropathy.

FIG. 26 Morphogen OP-1 dramatically reduces the serum creatinine level, while ACEI elalapril reduces the serum creatinine level at a lesser degree in the Alloxan-induced rat model of diabetic nephropathy.

FIG. 27 Morphogen OP-1 and ACEI enalapril both reduce the preteinuria level. However, conjoint administration of morphogen and ACEI synergistically reduce the preteinuria level in the Alloxan-induced rat model of diabetic nephropathy.

FIG. 28 Comparison of percent amino acid sequence homology/similarity and percent identity within the C-terminal seven cysteine skeletons of various representative members of the TGF-β superfamily proteins, using OP-1 as the reference sequence. The percent homologies recited in the figure are based on aligning the sequences using the MegaAlign Program (DNAstar, Inc.).

### DETAILED DESCRIPTION OF THE INVENTION

I. Overview

The present invention depends, in part, upon the surprising discovery that conjoint administration of ACE inhibitors with certain protein-based morphogens to subjects in, or at risk of, chronic renal failure, can reduce mortality and/or morbidity rates, and prevent, inhibit, delay or alleviate the progressive loss of renal function which characterizes chronic renal failure. Alternatively, or in addition, conjoint administration of ACE inhibitors with the morphogens of the present invention can prevent, inhibit or delay the progressive loss of functional nephron units and the progressive decline in glomerular filtration rate (GFR) which slowly but inevitably leads to the need for renal replacement therapy (i.e., renal transplant or chronic dialysis) or death. In preferred embodiments, the therapeutic agents of the invention are members of the osteogenic protein/bone morphogenetic protein (OP/BMP) family within the TGF-β superfamily of proteins, and inhibitors of the ACE family of proteins.

The present invention also contemplates conjoint administration of Angiotensin II Receptor Antagonists/Blockers (AIIRAs) with certain protein-based morphogens to subjects in, or at risk of, chronic renal failure, in order to reduce mortality and/or morbidity rates, and to prevent, inhibit, delay or alleviate the progressive loss of renal function which characterizes chronic renal failure. Alternatively, or in addition, conjoint administration of angiotensin II receptor blockers with the morphogens of the present invention can prevent, inhibit or delay the progressive loss of functional nephron units and the progressive decline in glomerular filtration rate (GFR) which slowly but inevitably leads to the need for renal replacement therapy (i.e., renal transplant or chronic dialysis) or death. In preferred embodiments, the therapeutic agents of the invention are members of the osteogenic protein/bone morphogenetic protein (OP/BMP) family within the TGF-β superfamily of proteins, and angiotensin II receptor blockers.

II. Definitions

In order to more clearly and concisely point out the subject matter of the claimed invention, the following definitions are provided for specific terms used in the following written description and appended claims.

OP/BMP morphogen. As used herein, the term “OP/BMP morphogen” means a polypeptide, or a functional variant of a polypeptide, comprising at least the C-terminal six- or seven-cysteine domain of a mammalian protein selected from the group consisting of OP-1, OP-2, OP-3, BMP2, BMP3, BMP4, BMP5, BMP6, BMP9, and proteins which exhibit at least 65% or, more preferably, 70%, 75% 80%, 85%, 90%, or 95% amino acid sequence homology, or at least 50%, more preferably 55%, 60%, 70%, 80%, 90%, 99% or more identity, with the amino acid sequence of the seven-cysteine domain of any one of the morphogens described above, such as human OP-1 (SEQ ID NO: 1); and which is (a) capable of inducing chondrogenesis in the Reddi-Sampath ectopic bone assay (Sampath and Reddi (1981), Proc. Natl. Acad. Sci. (USA) 78: 7599-7603) or a substantially equivalent assay, (b) capable of significantly preventing, inhibiting, delaying or alleviating the progressive loss of renal function in a standard animal model of chronic renal failure, or (c) capable of causing a clinically significant improvement in a standard marker of renal function when administered to a mammal in, or at risk of, chronic renal failure.

As used herein, “amino acid sequence homology” or a percentage “homology” between two amino acid sequences is understood herein to include both amino acid sequence identity and conserved substitution. Thus, as used herein, a percentage “homology” between two amino acid sequences indicates the percentage of amino acid residues which are identical or are conserved substitution between the sequences. “Conservative substitutions” of amino acids fulfill the criteria defined for an “accepted point mutation” in Dayhoff et al. (1978), Atlas of Protein Sequence and Structure Vol. 5 (Suppl. 3), pp. 354-352, Natl. Biomed. Res. Found., Washington, D.C. Thus, “conservative substitutions” are residues that are physically or functionally similar to the corresponding reference residues, having similar size, shape, electric charge, and/or chemical properties such as the ability to form covalent or hydrogen bonds, or the like. Examples of preferred conservative substitutions include the substitution of one amino acid for another with similar characteristics, e.g., substitutions within the following groups: (a) Ser, Thr, Pro, Ala, Gly; (b) Asn, Asp, Glu, Gln; (c) His, Arg, Lys; (d) Met, Ile, Leu, Val; (e) Phe, Tyr, Trp. In a most preferred embodiment, conservative substitutions include the substitution of one amino acid for another within the following groups: (a) glycine, alanine; (b) valine, isoleucine, leucine; (c) aspartic acid, glutamic acid; (d) asparagine, glutamine; (e) serine, threonine; (f) lysine, arginine, histidine; and (g) phenylalanine, tyrosine. See FIG. 84 of Dayhoff et al. (1978), Atlas of Protein Sequence and Structure Vol. 5 (Suppl. 3), pp. 354-352, Natl. Biomed. Res. Found., Washington, D.C. The term “conservative substitution” or “conservative variation” also includes the use of a substituted amino acid in place of an unsubstituted parent amino acid in a given polypeptide chain, provided that the resulting substituted polypeptide chain also has therapeutic efficacy in the present invention.

As used herein, a therapeutic agent (morphogen and/or ACEI) of the invention is said to have “therapeutic efficacy,” and an amount of the agent is said to be “therapeutically effective,” if administration of that amount of the agent is sufficient to cause a clinically significant improvement in a standard marker of renal function when administered to a mammalian subject (e.g., a human patient) in, or at risk of, chronic renal failure. Such markers of renal function are well known in the medical literature and include, without being limited to, rates of increase in BUN levels, rates of increase in serum creatinine, static measurements of BUN, static measurements of serum creatinine, glomerular filtration rates (GFR), ratios of BUN/creatinine, serum concentrations of sodium (Na⁺), urine/plasma ratios for creatinine, urine/plasma ratios for urea, urine osmolarity, daily urine output, and the like (see, for example, Brenner and Lazarus (1994), in Harrison's Principles of internal Medicine, 13th edition, Isselbacher et al., eds., McGraw Hill Text, New York; Luke and Strom (1994), in Internal Medicine, 4th Edition, J. H. Stein, ed., Mosby-Year Book, Inc. St. Louis).

As used herein, “conjoint administration” means administration of two or more agents to a subject of interest as part of a single therapeutic regimen. The administration(s) can be either simultaneous or sequential, i.e., administering one agent followed by administering of a second (and/or a third one, etc.) at a later time, as long as the agents administered co-exist in the subject being treated, or at least one agent will have the opportunity to act upon the same target tissues of other agents while said target tissues are still under the influence of said other agents. In a preferred embodiment, agents to be administered can be included in a single pharmaceutical composition and administered together. In another preferred embodiment, the agents are administered simultaneously, including through separate routes. In yet another preferred embodiment, one or more agents are administered continuously, while other agents are administered only at predetermined intervals (such as a single large dosage, or twice a week at smaller dosages, etc.). For example, the morphogens can be administered three times a week through direct injection, while the ACE inhibitors can be continuously released by an implant.

The route of administration can be the same or different, depending on needs or suitable methods of administration for each agent. Any suitable route of administration may be employed for providing the patient with effective dosages of an ACEI and a morphogen. For example, oral, rectal, parenteral, transdermal, subcutaneous, intramuscular, inhalation and the like may be employed. Dosage forms include tablets, troches, dispersions, suspensions, solutions, capsules, patches and the like. In certain embodiments, the morphogen can be administered via direct injection, while the ACE inhibitors can be administered through drinking water.

As used herein, “prevent” or “prevention” means reducing the probability/risk of developing a condition in a subject (cell, tissue, organ, or organism, etc.), or delaying the onset of a condition in the subject, or to lessening the severity of one or more symptoms of a condition that may develop in the subject, or any combination thereof.

Filtration. The kidneys' main function is to remove toxins (uremic wastes) that accumulate in the blood as a result of the body's metabolism. The body continuously uses digested elements from foods and stored nutrients to perform normal bodily functions. The by-products of nutrient metabolism and cell function are filtered from the blood by the kidneys, which excrete (discharge) wastes as urine.

Every day, approximately 200 liters of blood flow to the kidneys where 2 liters of waste are filtered out.

BUN and Creatinine. The concentration in the blood (blood level) of blood urea nitrogen (BUN), known as urea, and creatinine (Cr) can be measured by routine laboratory tests. BUN and creatinine levels indicate the general function of the kidneys. BUN is a metabolic by-product of protein-rich food such as meat, poultry, and certain vegetables. BUN is filtered out of the blood by the kidneys and excreted in the urine. Creatinine is continuously generated by normal cell metabolism within the muscles. Creatinine is also filtered out of the blood by the kidneys and excreted in the urine.

The amounts of BUN and creatinine in the blood are equal to the amount excreted by the kidneys. The blood levels of BUN and Cr remain unchanged unless there is sudden deterioration of renal (i.e., kidney) function. If the kidneys are suddenly unable to function, BUN and Cr increase daily. This condition is known as acute renal failure. Chronic renal failure is a condition distinguished by a gradual increase in BUN and Cr over a long period of time.

Measurement of Kidney Function. When renal function decreases, blood levels of Cr and BUN increase because the kidneys are unable to clean the blood effectively. Factors not related to the kidneys also impact BUN and Cr levels. Creatinine, in particular, is affected by age, sex, weight, and muscle mass.

Renal function is measured to evaluate the rate at which both kidneys are able to clean the blood. To measure renal function, a 24-hour urine sample must be collected. It is of importance that the 24-hour sample is complete (i.e., no urine is missing), so that true renal function will not be underestimated.

The amount of Cr in the urine sample is compared to the blood level of Cr. This figure is known as creatinine clearance (CrCl), the rate at which both kidneys clean the blood. The normal CrCl is about 90 to 130 milliliters per minute (mL/min). Many people gradually lose renal function as they age. Alternative renal function measurements rely on tables or formulas that take into consideration age, body weight, sex, and blood creatinine.

Some health care facilities in the United States offer the Glofil-125 assay to evaluate renal function. Sodium iothalamate 1-125 (a radiopharmaceutical) is injected into the skin, and blood and urine samples are obtained to determine renal function. The test is easy to perform, is more sensitive than blood creatinine measurements, and provides results within 2 to 3 hours. Measurements of renal function determine the severity of kidney impairment. It is important to monitor renal function over time to document the rate of deterioration or improvement with treatment.

Glomerular Filtration Rate (GFR). The “glomerular filtration rate” or “GFR” is proportional to the rate of clearance into urine of a plasma-borne substance which is not bound by serum proteins, is freely filtered across glomeruli, and is neither secreted nor reabsorbed by the renal tubules. Thus, as used herein, GFR preferably is defined by the following equation.
GFR=U_{conc}V/P_{conc}
where U_{conc} is the urine concentration of the marker, P_{conc} is the plasma concentration of the marker, and V is the urine flow rate in ml/min. Optionally, GFR is corrected for body surface area. Thus, the GFR values used herein may be regarded as being in units of ml/min/1.73 m².

The preferred measure of GFR is the clearance of insulin but, because of the difficulty of measuring the concentrations of this substance, the clearance of creatinine is typically used in clinical settings. For example, for an average size, healthy human male (70 kg, 20-40 yrs), a typical GFR measured by creatinine clearance is expected to be approximately 125 ml/min with plasma concentrations of creatinine of 0.7-1.5 mg/dL. For a comparable, average size woman, a typical GFR measured by creatinine clearance is expected to be approximately 115 ml/min with creatinine levels of 0.5-1.3 mg/dL. During times of good health, human GFR values are relatively stable until about age 40, when GFR typically begins to decrease with age. For subjects surviving to age 85-90, GFR may be reduced to 50% of the comparable values at 40.

Expected Glomerular Filtration Rate (GFRₑₓₚ). An estimate of the “expected GFR” or “GFRxp” may be provided based upon considerations of a subject's age, weight, sex, body surface area, and degree of musculature, and the plasma concentration of some marker compound (e.g., creatinine) as determined by a blood test. Thus, as an example, an expected GFR or GFRₑₓₚ maybe estimated as:
GFRₑₓₚ=(140−age)×weight (kg)/[72×P_{conc} (mg/dl)]

This estimate does not take into consideration such factors as surface area, degree of musculature, or percentage body fat. Nonetheless, using plasma creatinine levels as the marker, this formula has been employed for human males as an inexpensive means of estimating GFR. Because creatinine is produced by striated muscle, the expected GFR or GFRₑₓₚ of human female subjects is estimated by the same equation multiplied by 0.85 to account for expected differences in muscle mass. (See Lemann, et al. (1990) Am. J. Kidney Dis. 16 (3): 236).

Broad Cast. Microscopic examination of urinary sediment for the presence of formed elements is a standard procedure in urinalysis. Amongst the formed elements which may be present in urine are cylindrical masses of agglutinated materials that typically represent a mold or “cast” of the lumen of a distal convoluted tubule or collecting tubule. In healthy human subjects, such casts typically have a diameter of 15-25 μm. In subjects with chronic renal failure, however, hypertrophy of the tubules may result in the presence of “broad casts” or “renal failure casts” which are 2-6 times the diameter of normal casts and often have a homogeneous waxy appearance. Thus, as used herein, a “broad cast” means a urinary sediment cast having a diameter of 2-6 times normal, or about 30-150 μm for human casts.

Chronic. As used herein with respect to clinical indications such as urinary casts, measured GFR, or other markers of renal function, “chronic” means persisting for a period of at least three, and more preferably, at least six months. Thus, for example, a subject with a measured GFR chronically below 50% of GFRₑₓₚ is a subject in which the GFR has be enmeasured and found to be below 50% of GFRₑₓₚ in at least two measurements separated by at least three, and more preferably, by at least six months, and for which there is no medically sound reason to believe that GFR was substantially (e.g., 10%) higher during the intervening period.

Subjects in, or at risk of, chronic renal failure. As used herein, a subject is said to be in, or at risk of chronic renal failure, or at risk of the need for renal replacement therapy, if the subject is reasonably expected to suffer a progressive loss of renal function associated with progressive loss of functioning nephron units. Whether a particular subject is in, or at risk of, chronic renal failure is a determination which may routinely be made by one of ordinary skill in the relevant medical or veterinary art. Subjects in, or at risk of, chronic renal failure, or at risk of the need for renal replacement therapy, include but are not limited to the following: subjects which may be regarded as afflicted with chronic renal failure, end-stage renal disease, chronic diabetic nephropathy, hypertensive nephrosclerosis, chronic glomerulonephritis, hereditary nephritis, and/or renal dysplasia; subjects having a biopsy indicating glomerular hypertrophy, tubular hypertrophy, chronic glomerulosclerosis, and/or chronic tubulointerstitial sclerosis; subjects having an ultrasound, NMR, CAT scan, or other non-invasive examination indicating renal fibrosis; subjects having an unusual number of broad casts present in urinary sediment; subjects having agar which is chronically less than about 50%, and more particularly less than about 40%, 30% or 20%, of the expected GFR for the subject; human male subjects weighing at least about 50 kg and having a GFR which is chronically less than about 50 ml/min, and more particularly less than about 40 ml/min, 30 ml/min or 20 ml/min; human female subjects weighing at least about 40 kg and having a GFR which is chronically less than about 40 ml/min, and more particularly less than about 30 ml/min, 20 ml/min or 10 ml/min; subjects possessing a number of functional nephron units which is less than about 50%, and more particularly less than about 40%, 30% or 20%, of the number of functional nephron units possessed by a healthy but otherwise similar subject; subjects which have a single kidney; and subjects which are kidney transplant recipients.

III. Description of the Preferred Embodiments

A. Therapeutic Agents

The morphogens of the present invention are naturally occurring proteins, or functional variants of naturally occurring proteins, in the osteogenic protein/bone morphogenetic protein (OP/BMP) family within the TGF-β superfamily of proteins.

The ACE inhibitors of the invention are generally small organic molecules. “Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules.

(i) OP/BMP Family of Morphogens

The “OP/BMP family” of proteins forms a distinct subgroup, within the loose evolutionary grouping of sequence-related proteins known as the TGF-β superfamily. Members of this protein family comprise secreted polypeptides that share common structural features, and that are similarly processed from a pro-protein to yield a carboxy-terminal mature protein. This family of proteins is also referred to as morphogens. As noted above, a protein is morphogenic as defined herein if it induces the developmental cascade of cellular and molecular events that culminate in the formation of new, organ-specific tissue.

It has been discovered that morphogens enhance survival of neurons and maintain neural pathways. As described herein, morphogens are capable of enhancing survival of neurons, stimulating neuronal CAM expression, maintaining the phenotypic expression of differentiated neurons, inducing the redifferentiation of transformed cells of neural origin, and stimulating axonal growth over breaks in neural processes, particularly large gaps in axons. Morphogens also protect against tissue destruction associated with immunologically related nerve tissue damage. In addition, morphogens may be used as part of a method for monitoring the viability of nerve tissue in a mammal.

In a preferred embodiment, a morphogen is a dimeric protein, each polypeptide component of which has a sequence that corresponds to, or is functionally equivalent to, at least the conserved C-terminal six or seven cysteine skeleton of human OP-1, included in SEQ ID NO: 2, and/or which shares 70% amino acid sequence homology or 50% identity with OP-1 in this region. The morphogens are generally competent to induce a cascade of events including the following, in a morphogenically permissive environment: stimulating proliferation of progenitor cells; stimulating the differentiation of progenitor cells; stimulating the proliferation of differentiated cells; and supporting the growth and maintenance of differentiated cells. Under appropriate conditions, morphogens are also competent to induce redifferentiation of cells that have undergone abnormal differentiation. Details of how the morphogens useful in this invention were identified, as well as a description on how to make, use and test them for morphogenic activity are disclosed in numerous publications, including U.S. Pat. Nos. 5,011,691 and 5,266,683, and the international patent application publications WO 92/15323; WO 93/04692; and WO 94/03200, each of which are incorporated by reference herein. As disclosed therein, the morphogens can be purified from naturally sourced material or recombinantly produced from prokaryotic or eukaryotic host cells, using the genetic sequences disclosed therein. Alternatively, novel morphogenic sequences can be identified following the procedures disclosed therein.

The naturally occurring morphogens share substantial amino acid sequence homology in their C-terminal sequences (sharing, e.g., a six or seven cysteine skeleton sequence). Typically, a naturally occurring morphogen is translated as a precursor, having an N-terminal signal peptide sequence, typically less than about 35 residues in length, followed by a “pro” domain that is cleaved to yield the mature polypeptide, which includes the biologically active C-terminal skeleton sequence. The signal peptide is cleaved rapidly upon translation, at a cleavage site that can be predicted in a given sequence using the method of Von Heijne, Nucleic Acids Research 14: 4683-4691 (1986). The “pro” domain is variable both in sequence and in length, ranging from approximately 200 to over 400 residues. The pro domain is cleaved to yield the “mature” C-terminal domain of approximately 115-180 residues, which includes the conserved six- or seven-cysteine C-terminal domain of 97-106 residues. The pro polypeptide typically is about three times larger than the fully processed, mature C-terminal polypeptide. Under native conditions, the protein is secreted as a mature dimer and the cleaved pro polypeptide is thought to remain associated therewith to form a protein complex, presumably to improve the solubility of the mature dimeric protein. The complexed form of a morphogen is generally observed to be more soluble than the mature form under physiological conditions.

As used herein, the “pro form” of an OP/BMP family member refers to a protein comprising a folded pair of polypeptides, each comprising a pro domain in either covalent or noncovalent association with the mature domains of the OP/BMP polypeptide. The pro form appears to be the primary form secreted from cultured mammalian cells. The “mature form” of the protein refers to mature C-terminal domain which is not associated, either covalently or noncovalently, with the pro domain. Any preparation of OP-1 is considered to contain mature form when the amount of pro domain in the preparation is no more than 5% of the amount of “mature” C-terminal domain.

Natural-sourced morphogenic protein in its mature, native form, is typically a glycosylated dimer, having an apparent molecular weight of about 30-36 kDa as determined by SDS-PAGE. When reduced, the 30 kDa protein gives rise to two glycosylated polypeptide subunits having apparent molecular weights in the range of about 16 kDa and about 18 kDa. The unglycosylated dimeric protein, which also has morphogenic activity, typically has an apparent molecular weight in the range of about 27 kDa. When reduced, the 27 kDa protein gives rise to two unglycosylated polypeptides having molecular weights typically in the range of about 14 kDa to about 16 kDa.

OP/BMP family members useful herein include any of the known naturally occurring native proteins including allelic, phylogenetic counterpart and other variants thereof, whether naturally sourced or biosynthetically produced (e.g., including “muteins” or “mutant proteins”), as well as new, active members of the OP/BMP family of proteins. Particularly useful sequences include those comprising the C-terminal seven cysteine domains of mammalian, preferably human, OP-1, OP-2, OP-3, BMP2, BMP3, BMP4, BMP5, BMP6, BMP8 and BMP9. Other proteins useful in the practice of the invention include active forms of DPP, Vgl, Vgr-1, 60A, GDF-1, GDF-3, GDF-5, GDF-6, GDF-7, BMP10, BMP11; BMP13, BMP15, UNIVIN, NODAL, SCREW, ADMP or NURAL and amino acid sequence variants thereof. In one preferred embodiment, the morphogens of the invention are selected from any one of OP-1, OP-2, OP-3, BMP2, BMP3, BMP4, BMP5, BMP6, and BMP9.

In preferred embodiments, each of the polypeptide subunits of a dimeric morphogenic protein as defined herein comprises an amino acid sequence sharing a defined relationship with an amino acid sequence of a reference morphogen. In one embodiment, preferred morphogenic polypeptide chains share a defined relationship with a sequence present in morphogenically active full-length human OP-1, SEQ ID NO: 3. However, any one or more of the naturally occurring or biosynthetic morphogenic proteins disclosed herein similarly could be used as a reference sequence. Preferred morphogenic polypeptide chains share a defined relationship with at least the C-terminal six cysteine skeleton of human OP-1, residues 335-431 of SEQ ID NO: 3 (or residues 38-139 of SEQ ID NO: 1). Preferably, morphogenic proteins share a defined relationship with at least the C-terminal seven cysteine skeleton of human OP-1, residues 330-431 of SEQ ID NO: 3 (or residues 38-139 of SEQ ID NO: 1).

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric morphogen protein, including their ability to form such intra- or inter-chain disulfide bonds as may be necessary for morphogenic activity. For example naturally occurring morphogens have been described in which at least one internal deletion (of one residue; BMP2) or insertion (of four residues; GDF-1) is present but does not abrogate biological activity. Functionally equivalent sequences further include those wherein one or more amino acid residues differ from the corresponding residue of a reference sequence, e.g., the C-terminal seven cysteine skeleton of human OP-1, provided that this difference does not destroy tissue morphogenic activity. Accordingly, conservative substitutions of corresponding amino acids in the reference sequence are preferred. Amino acid residues that are “conservative substitutions” for corresponding residues in a reference sequence are those that are physically or functionally similar to the corresponding reference residues, e.g., that have similar size, shape, electric charge, chemical properties including the ability to form covalent or hydrogen bonds, or the like. Particularly preferred conservative substitutions are those fulfilling the criteria defined for an accepted point mutation in Dayhoff, et al., 5 ATLAS OF PROTEIN SEQUENCE AND STRUCTURE, Suppl. 3, ch. 22 pp. 354-352 (1978), Natl. Biomed. Res. Found., Washington, D.C. (supra), the teachings of which are incorporated by reference herein. The term “conservative substitution” also includes the use of a synthetic or derivatized amino acid in place of the corresponding natural parent amino acid, provided that antibodies raised to the resulting variant polypeptide also immunoreact with the corresponding naturally sourced morphogen polypeptide.

Among the morphogens useful in this invention are proteins originally identified as osteogenic proteins, such as the OP-1, OP-2 and CBMP2 proteins, as well as amino acid sequence-related proteins such as DPP (from Drosophila), Vgl (from Xenopus), Vgr-1 (from mouse, see U.S. Pat. No. 5,011,691 to Oppermann et al.), GDF-1 (from mouse, see Lee (1991) PNAS 88: 4250-4254), all of which are presented in Table II), and the recently identified 60A protein (from Drosophila, see Wharton et al. (1991) PNAS 88: 9214-9218). As mentioned before, the members of this family, which include members of the TGF-β super-family of proteins, share substantial amino acid sequence homology in their C-terminal regions. The proteins are translated as a precursor, having an N-terminal signal peptide sequence, typically less than about 30 residues, followed by a “pro” domain that is cleaved to yield the mature sequence. The signal peptide is cleaved rapidly upon translation, at a cleavage site that can be predicted in a given sequence using the method of Von Heijne ((1986) Nucleic Acids Research 14:4683-4691.)

Table I below summarizes various naturally occurring members of the OP/BMP family identified to date, including their nomenclature as used herein, their Sequence listing references, and publication sources for the amino acid sequences for the full length proteins not included in the Sequence listing. Each of the generic terms set forth in Table I is intended and should be understood to embrace the therapeutic effective proteins expressed from nucleic acids encoding the identified sequence mentioned below and set forth in the Sequence listing, or an active fragment or precursor thereof, or a functional equivalent thereof such as a naturally occurring or biosynthetic variant. Naturally occurring variants include allelic variant forms isolated from other individuals of a single biological species, as well as species variants (homologous) isolated from phylogenetically distinct biological species.

TABLE I
Exemplary Morphogens

| | |
|---|---|
| “OP-1” | Refers generically to the group of morphogenically active proteins expressed |
| | from part or all of a DNA sequence encoding OP-1 protein, |
| | including allelic and species variants thereof, e.g., human OP-1 |
| | (“hOP-1”, SEQ ID NOs: 1, mature protein amino acid sequence), or |
| | mouse OP-1 (“mOP-1”, SEQ ID NO: 4, mature protein amino acid |
| | sequence.) The conserved seven cysteine skeleton is defined by |
| | residues 38 to 139 of SEQ ID NOs: 1 and 4. The cDNA sequences |
| | and the amino acids encoding the full length proteins are provided in |
| | SEQ ID NOs: 17 and 3 (hOP-1) and SEQ ID NOs: 18 and 19 (mOP- |
| | 1). The mature proteins are defined by residues 293-431 (hOP-1) and |
| | 292-430 (mOP-1). The “pro” regions of the proteins, cleaved to yield |
| | the mature, morphogenically active proteins are defined essentially by |
| | residues 30-292 (hOP-1) and residues 30-291 (mOP-1). |
| “OP-2” | refers generically to the group of active proteins expressed from part |
| | or all of a DNA sequence encoding OP-2 protein, including allelic and |
| | species variants thereof, e.g., human OP-2 (“hOP-2”, SEQ ID No: 5, |
| | mature protein amino acid sequence) or mouse OP-2 (“mOP-2”, SEQ |
| | ID No: 6, mature protein amino acid sequence). The conserved seven |
| | cysteine skeleton is defined by residues 38 to 139 of SEQ ID NOs: 5 |
| | and 6. The cDNA sequences and the amino acids encoding the full |
| | length proteins are provided in SEQ ID NOs: 20 and 21 (hOP-2) and |
| | SEQ ID NOs: 22 and 23 (mOP-2). The mature proteins are defined |
| | essentially by residues 264-402 (hOP-2) and 261-399 (mOP-2). The |
| | “pro”regions of the proteins, cleaved to yield the mature, |
| | morphogenically active proteins likely are defined essentially by |
| | residues 18-263 (hOP-2) and residues 18-260 (mOP-2). Another |
| | cleavage site also occurs 21 residues upstream for both OP-2 proteins. |
| “CBMP2” | refers generically to the morphogenically active proteins expressed |
| | from a DNA sequence encoding the CBMP2 proteins, including |
| | allelic and species variants thereof, e.g., human CBMP2A |
| | (“CBMP2A(fx)”), SEQ ID NO: 10) or human CBMP2B DNA |
| | (“CBMP2B(fx)”), SEQ ID NO: 11). The amino acid sequence for the |
| | full length proteins, referred to in the literature as BMP2A and |
| | BMP2B, or BMP2 and BMP4, appear in Wozney, et al. (1988) |
| | Science 242: 1528-1534, the content of which is incorporated by |
| | reference herein. The pro-domain for BMP2 (BMP2A) likely includes |
| | residues 25-248 or 25-282; the mature protein, residues 249-396 or |
| | 283-396. The pro-domain for BMP4 (BMP2B) likely includes |
| | residues 25-256 or 25-292; the mature protein, residues 257-408 or |
| | 293-408. |
| “DPP(fx)” | refers to protein sequences encoded by the Drosophila DPP gene |
| | (DPP protein, see SEQ ID NO: 7) and defining the conserved seven |
| | cysteine skeleton. The amino acid sequence for the full length protein |
| | appears in Padgett, et al (1987) Nature 325: 81-84, the content of |
| | which is incorporated by reference herein. The pro-domain likely |
| | extends from the signal peptide cleavage site to residue 456; mature |
| | protein likely is defined by residues 457-588. The sequence of |
| | DPP(fx) is shown in Table II. |
| “Vg1(fx)” | refers to protein sequences encoded by the Xenopus Vg1 gene (Vg1 |
| | protein, see SEQ ID NO: 8) and defining the conserved seven cysteine |
| | skeleton. The amino acid sequence for the full length protein appears |
| | in Weeks (1987) Cell 51: 861-867, the content of which is |
| | incorporated by reference herein. The pro-domain likely extends from |
| | the signal peptide cleavage site to residue 246; the mature protein |
| | likely is defined by residues 247-360. The sequence of Vg1(fx) is |
| | shown in Table II. |
| ““Vgr-1(fx)” | refers to protein sequences encoded by the murine vgr-1 gene (Vgr-1 |
| | protein, see SEQ ID NO: 9) and defining the conserved seven cysteine |
| | skeleton. The amino acid sequence for the full length protein appears |
| | in Lyons, et al., (1989) PNAS 86: 4554-4558, the content of which is |
| | incorporated by reference herein. The pro-domain likely extends from |
| | the signal peptide cleavage site to residue 299; the mature protein |
| | likely is defined by residues 300-438. The sequence of Vgr-1(fx) is |
| | shown in Table II. |
| “GDF-1(fx)” | refers to protein sequences encoded by the human GDF-1 gene (GDF- |
| | 1 protein, see SEQ ID NO: 13) and defining the conserved seven |
| | cysteine skeleton. The amino acid sequence for the full length protein |
| | is provided in SEQ ID NO: 13. The pro-domain likely extends from |
| | the signal peptide cleavage site to residue 214; the mature protein |
| | likely is defined by residues 215-372. The sequence of GDF-1(fx) is |
| | shown in Table II. |
| “60A” | refers generically to the morphogenically active proteins expressed |
| | from part or all of a DNA sequence (from the Drosophila 60A gene) |
| | encoding the 60A proteins (see SEQ ID NO: 14). “60A(fx)” refers to |
| | the protein sequences defining the conserved seven cysteine skeleton |
| | (residues 354 to 455 of SEQ ID NO: 14). The pro-domain likely |
| | extends from the signal peptide cleavage site to residue 324; the |
| | mature protein likely is defined by residues 325-455. The sequence of |
| | 60A(fx) is shown in Table II. |
| “BMP3(fx)” | refers to protein sequences encoded by the human BMP3 gene (BMP3 |
| | protein, see SEQ ID NO: 12) and defining the conserved seven |
| | cysteine skeleton. The amino acid sequence for the full length protein |
| | appears in Wozney et al. (1988) Science 242: 1528-1534, the content |
| | of which is incorporated by reference herein. The pro-domain likely |
| | extends from the signal peptide cleavage site to residue 290; the |
| | mature protein likely, is defined by residues 291-472. The sequence of |
| | BMP3(fx) is shown in Table II. |
| “BMP5(fx)” | refers to protein sequences encoded by the human BMP5 gene (BMP5 |
| | protein, see SEQ ID NO: 15) and defining the conserved seven |
| | cysteine skeleton. The amino acid sequence for the full length protein |
| | appears in Celeste, et al. (1991) PNAS 87: 9843-9847, the content of |
| | which is incorporated by reference herein. The pro-domain likely |
| | extends from the signal peptide cleavage site to residue 316; the |
| | mature protein likely is defined by residues 317-454. The sequence of |
| | BMP5(fx) is shown in Table II. |
| “BMP6(fx)” | refers to protein sequences encoded by the human BMP6 gene (BMP6 |
| | protein, see SEQ ID NO: 16) and defining the conserved seven |
| | cysteine skeleton. The amino acid sequence for the full length protein |
| | appears in Celeste, et al. (1990) PNAS 87: 9843-9847, the content of |
| | which is incorporated by reference herein. The pro-domain likely |
| | includes extends from the signal peptide cleavage site to residue 374; |
| | the mature sequence likely includes residues 375-513. The sequence |
| | of BMP6(fx) is shown in Table II. |

The OP-2 proteins have an “additional” cysteine residue in this region (e.g., see residue 41 of SEQ ID NOs: 21 and 23), in addition to the conserved cysteine skeleton in common with the other proteins in this family. The GDF-1 protein has a four amino acid insert within the conserved skeleton (compare SEQ ID NO: 19 with SEQ ID NO: 13) but this insert likely does not interfere with the relationship of the cysteines in the folded structure. In addition, the CBMP2 proteins are missing one amino acid residue within the cysteine skeleton.

The morphogens are inactive when reduced, but are active as oxidized homodimers and when oxidized in combination with other morphogens of this invention (e.g., as heterodimers). Thus, as defined herein, a morphogen is a dimeric protein comprising a pair of polypeptide chains, wherein each polypeptide chain comprises at least the C-terminal six cysteine skeleton defined by residues 43-139 of SEQ ID NO: 1, including functionally equivalent arrangements of these cysteines (e.g., amino acid insertions or deletions which alter the linear arrangement of the cysteines in the sequence but not their relationship in the folded structure), such that, when the polypeptide chains are folded, the dimeric protein species comprising the pair of polypeptide chains has the appropriate three-dimensional structure, including the appropriate intra-or inter-chain disulfide bonds such that the protein is capable of acting as a morphogen as defined herein. Specifically, the morphogens generally are capable of all of the following biological functions in a morphogenically permissive environment: stimulating proliferation of progenitor cells; stimulating the differentiation of progenitor cells; stimulating the proliferation of differentiated cells; and supporting the growth and maintenance of differentiated cells, including the “redifferentiation” of transformed cells. In addition, it is also anticipated that these morphogens are capable of inducing redifferentiation of committed cells under appropriate environmental conditions.

The following publications disclose published morphogen polypeptide sequences, as well as relevant chemical and physical properties, of naturally occurring and/or synthetic morphogens: OP-1 and OP-2: U.S. Pat. No. 5,011,691, U.S. Pat. No. 5,266,683, Ozkaynak, et al., EMBO J. 9: 2085-2093 (1990); OP-3: WO 94/10203 (PCT US93/10520); BMP-2, BMP-3, and BMP-4: WO 88/00205, Wozney, et al., Science 242: 1528-1534 (1988); BMP-5 and BMP-6: Celeste, et al., PNAS 87: 9843-9847 (1991); Vgrl: Lyons, et al., PNAS 86: 45544558 (1989); DPP: Padgett, et al., Nature 325: 81-84 (1987); Vg-1: Weeks Cell 51: 861-867 (1987); BMP-9: WO 95/33830 (PCT/US95/07084); BMP-10: WO 94/26893 (PCT/US94/05290); BMP-11: WO 94/26892 (PCT/US94/05288); BMP-12: WO 95/16035 (PCT/US94/14030); BMP-13: WO 95/16035 (PCT/US94/14030); GDF-1: WO 92/00382 (PCT/US91/04096) and Lee, et al., PNAS 88:4250-4254 (1991); GDF-8: WO 94/21681 (PCT/US94/03019); GDF-9: WO 94/15966 (PCT/US94/00685); GDF-10: WO 95/10539 (PCT/US94/11440); GDF-11: WO 96/01845 (PCT/US95/08543); BMP-15: WO 96/36710 (PCT/US96/06540); MP121: WO 96/01316 (PCT/EP95/02552); GDF-5 (CDMP-1, MP52): WO 94/15949 (PCT/US94/00657) and WO 96/14335 (PCT/US94/12814) and WO 93/16099 (PCT/EP93/00350); GDF-6 (CDMP-2, BMP-13): WO 95/01801 (PCT/US94/07762) and WO 96/14335 and WO 95/10635 (PCT/US94/14030); GDF-7 (CDMP-3, BMP-12): WO 95/10802 (PCT/US94/07799) and WO 95/10635 (PCT/US94/14030). In another embodiment, useful proteins include biologically active biosynthetic constructs, including novel biosynthetic morphogenic proteins and chimeric proteins designed using sequences from two or more known morphogens. See also the biosynthetic constructs disclosed in U.S. Pat. No. 5,011,691 (e.g., COP-1, COP-3, COP-4, COP-5, COP-7, and COP-16). The disclosure of all cited references describing morphogens and other related proteins are incorporated herein by reference.

In certain preferred embodiments, useful morphogenic proteins include those in which the amino acid sequences comprise a sequence sharing at least 70% amino acid sequence homology (identity or conserved substitution), and preferably 80%, 85%, 90%, 95% or 99% homology, with a reference morphogenic protein selected from the exemplary naturally occurring morphogenic proteins listed herein. Preferably, the reference protein is human OP-1, and the reference sequence thereof is the C-terminal seven cysteine skeleton present in osteogenically active forms of human OP-1, residues 330-431 of SEQ ID NO: 3 (or residues 38-139 of SEQ ID NO: 1). Useful morphogenic proteins accordingly include allelic, phylogenetic counterpart and other variants of the preferred reference sequence, whether naturally occurring or biosynthetically produced (e.g., including “muteins” or “mutant proteins”), as well as novel members of the general morphogenic family of proteins including those set forth and identified above. Certain particularly preferred morphogenic polypeptides share at least 50% amino acid identity with the preferred reference sequence of human OP-1, or any of the other morphogens described above, still more preferably at least 55%, 60%, 65%, 70%, 80%, 85%, 90%, 95%, 99% or more amino acid identity therewith.

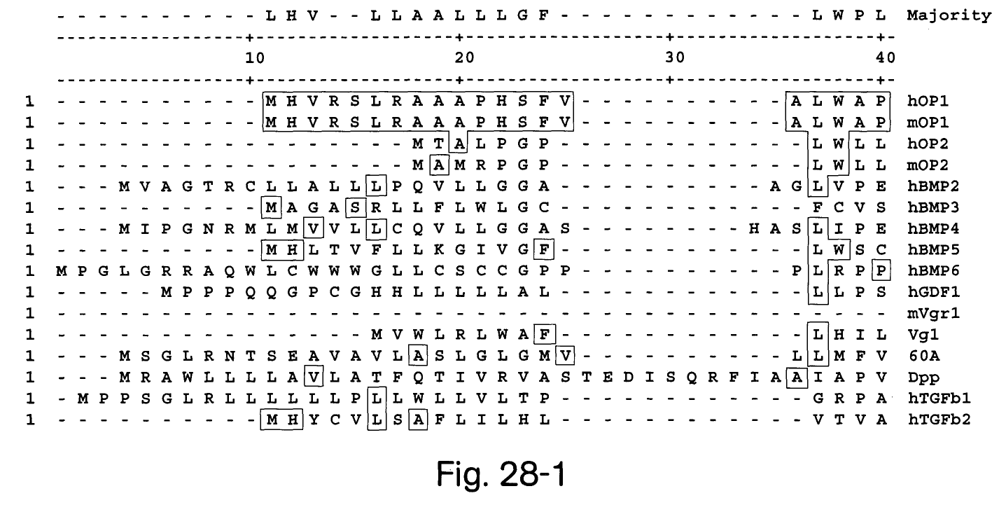

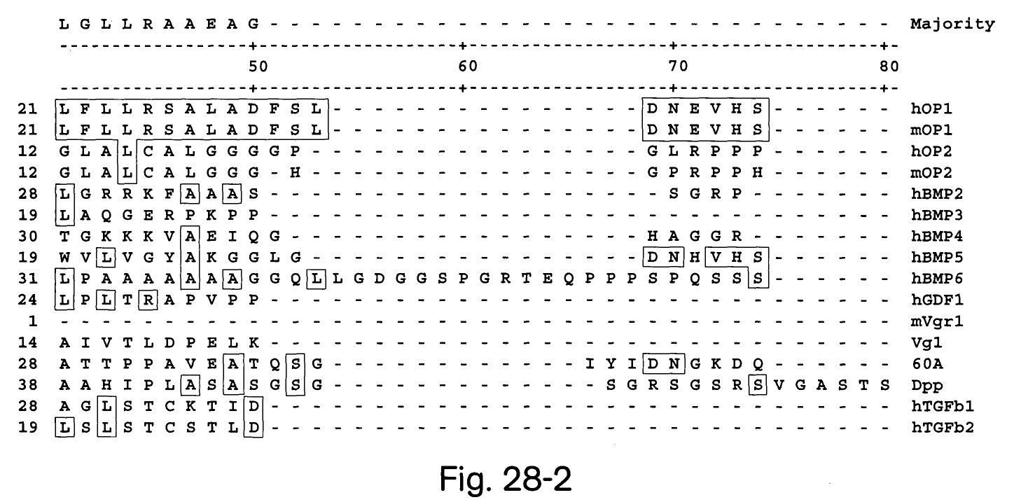

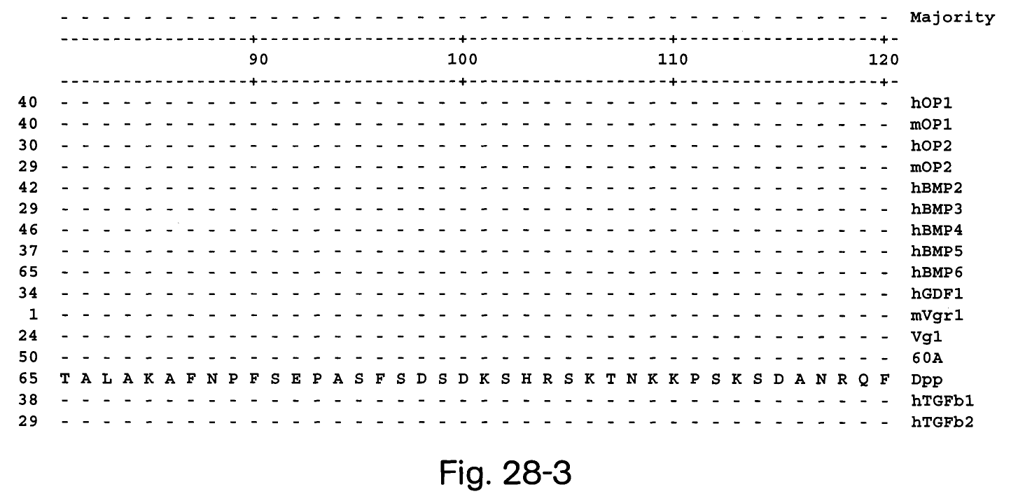

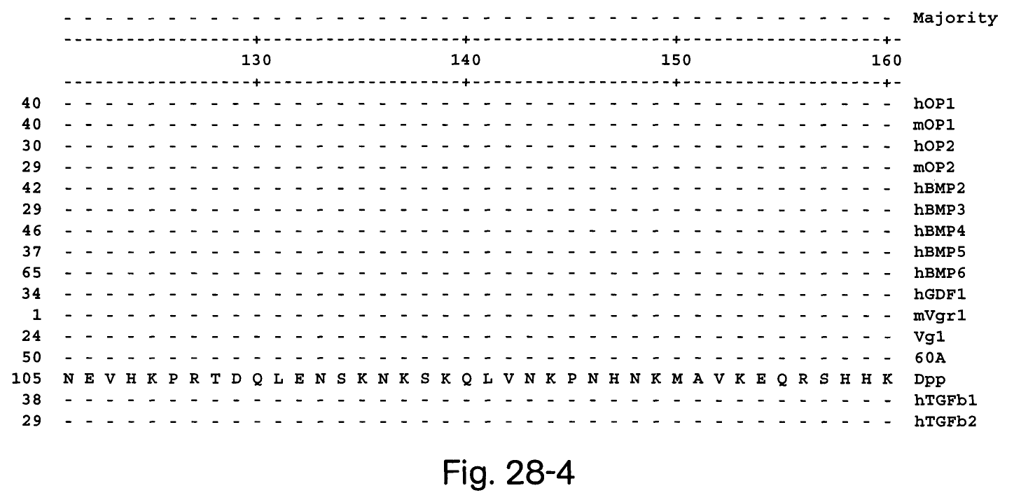

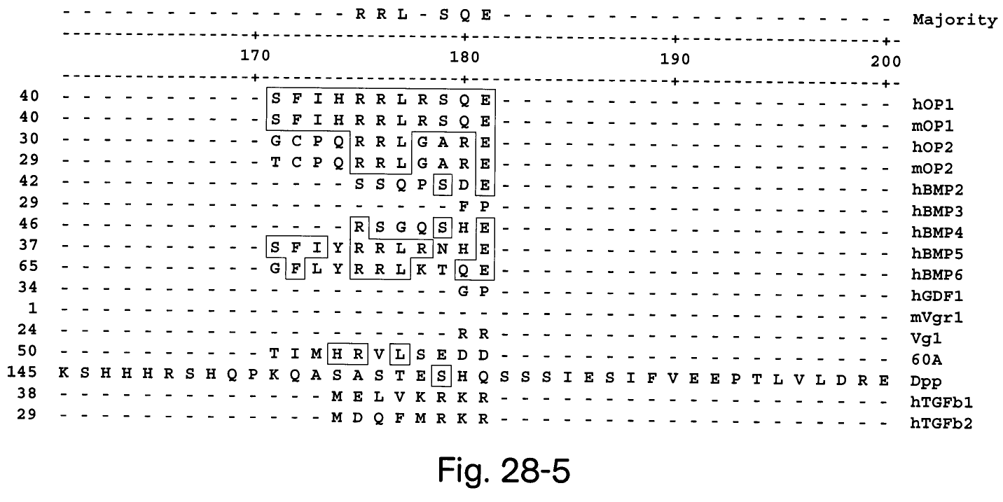

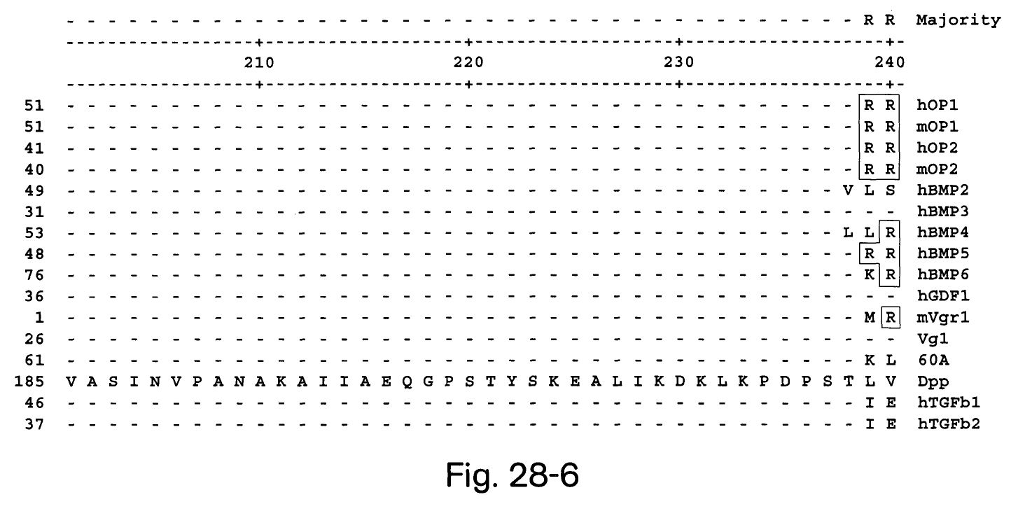

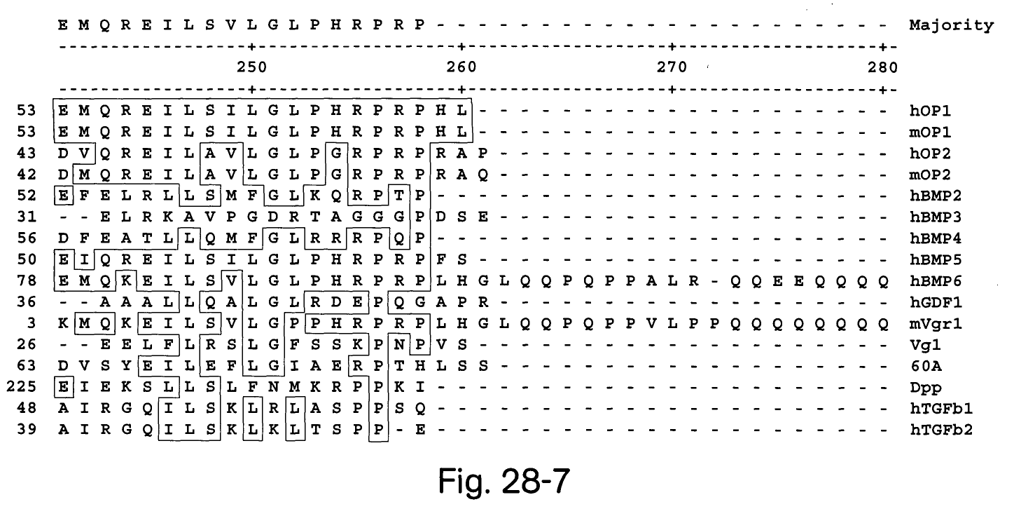

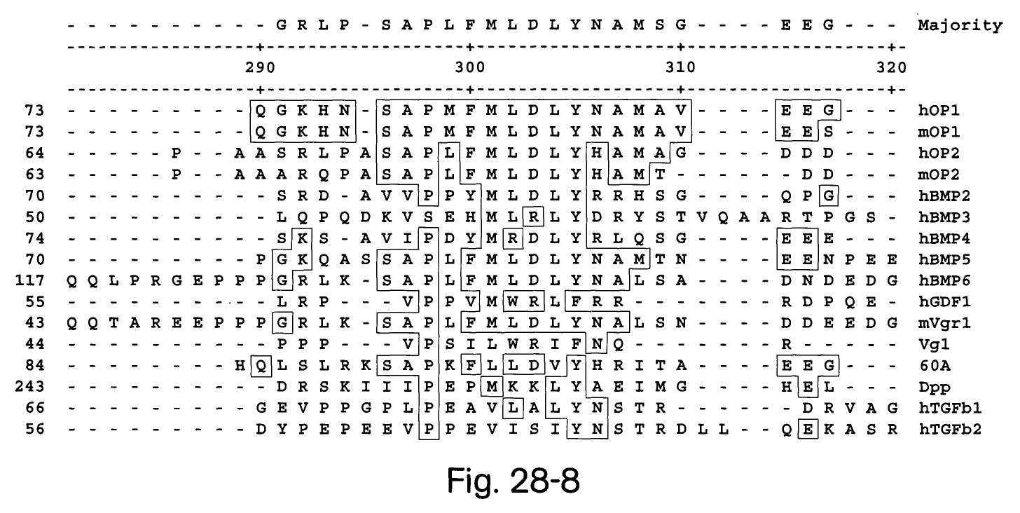

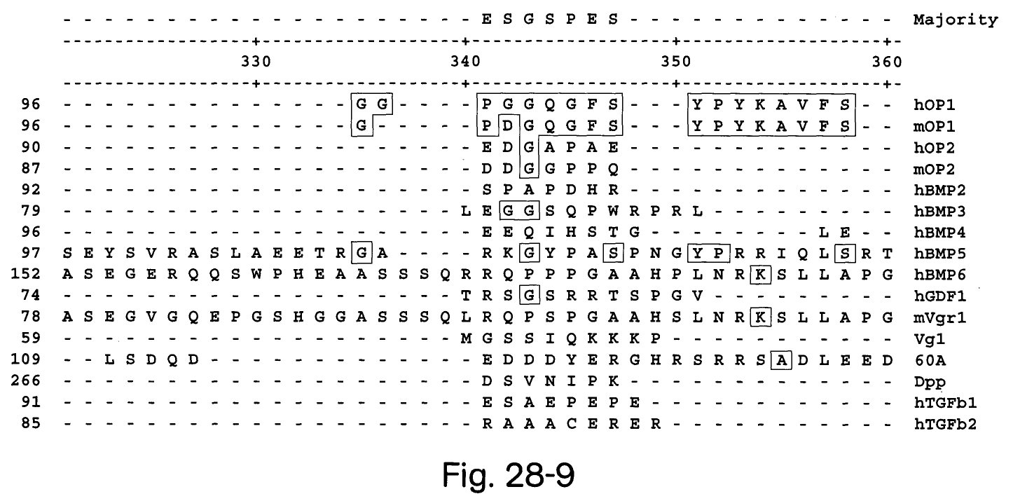

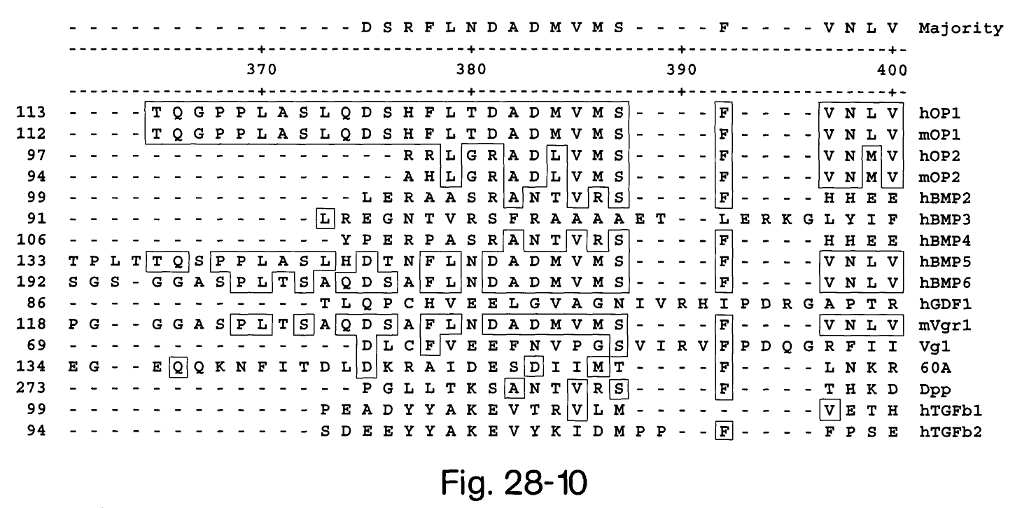

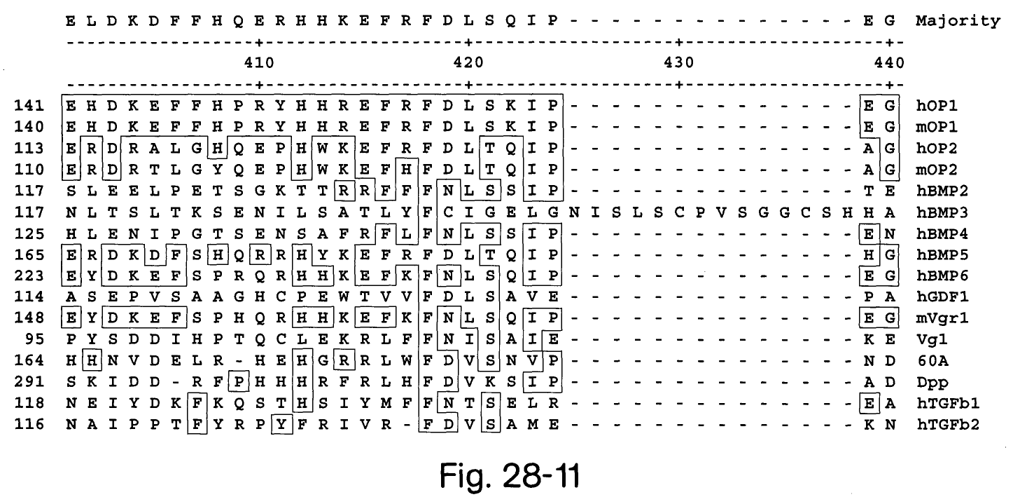

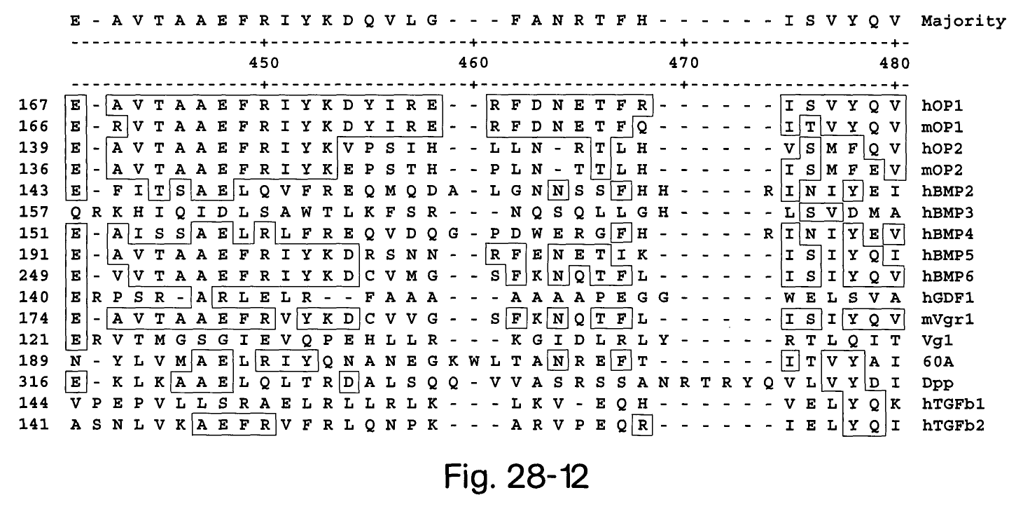

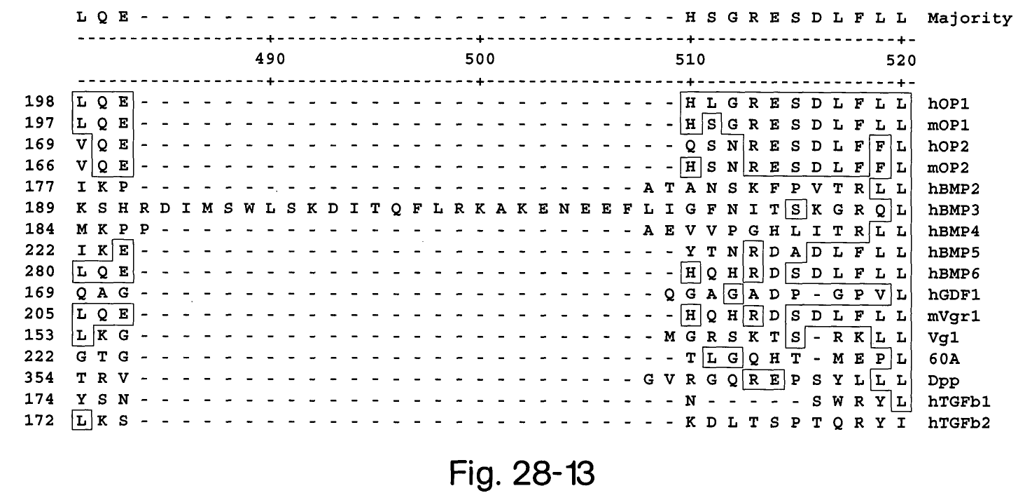

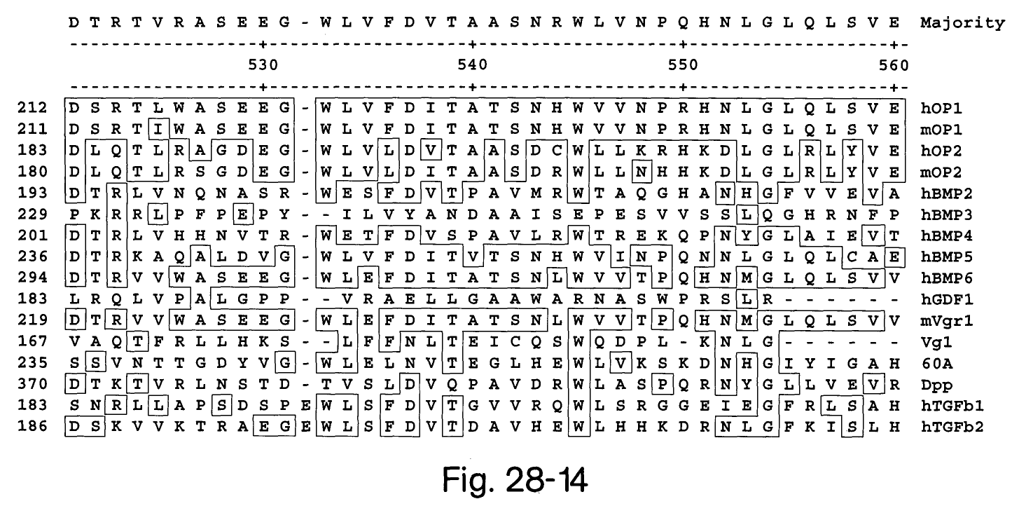

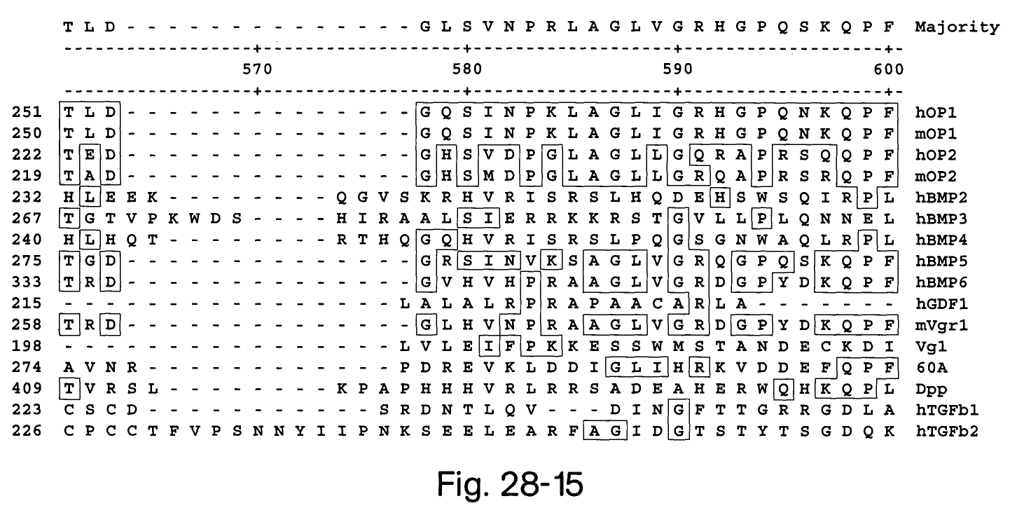

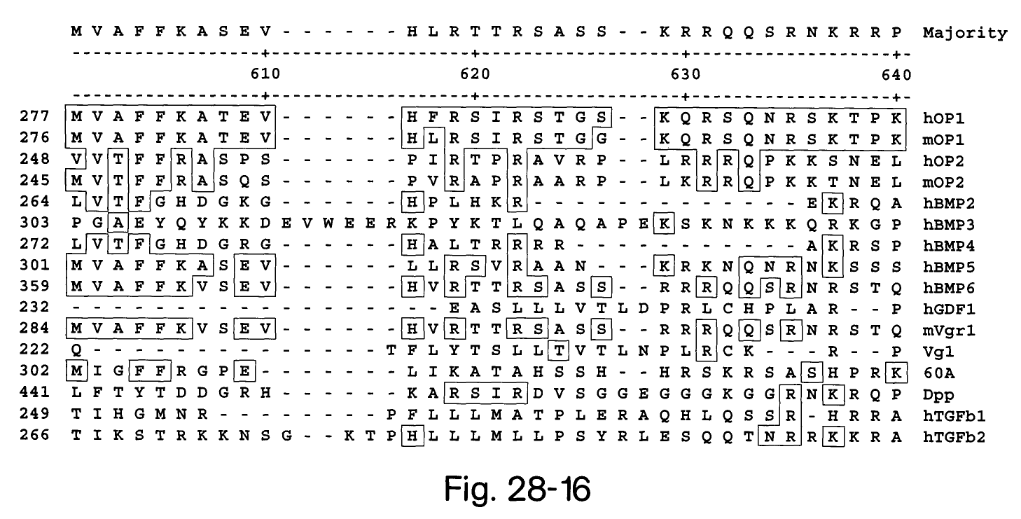

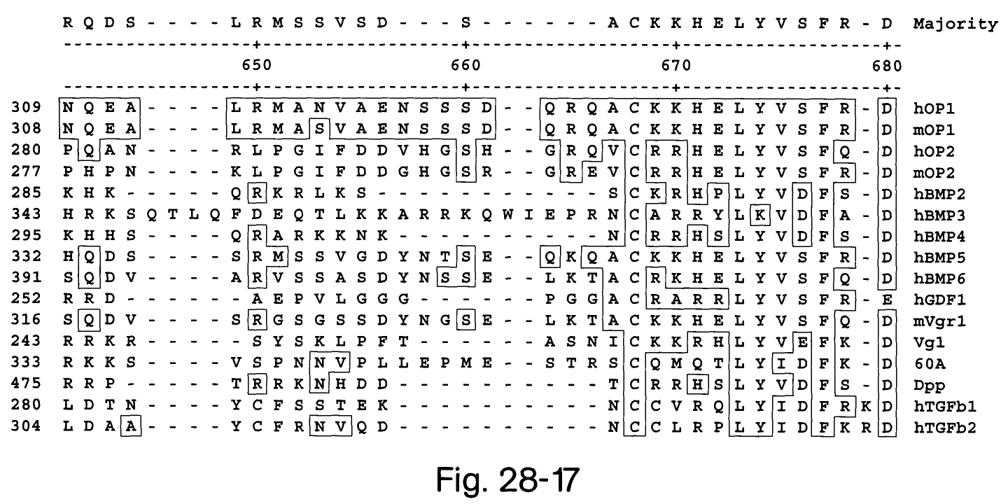

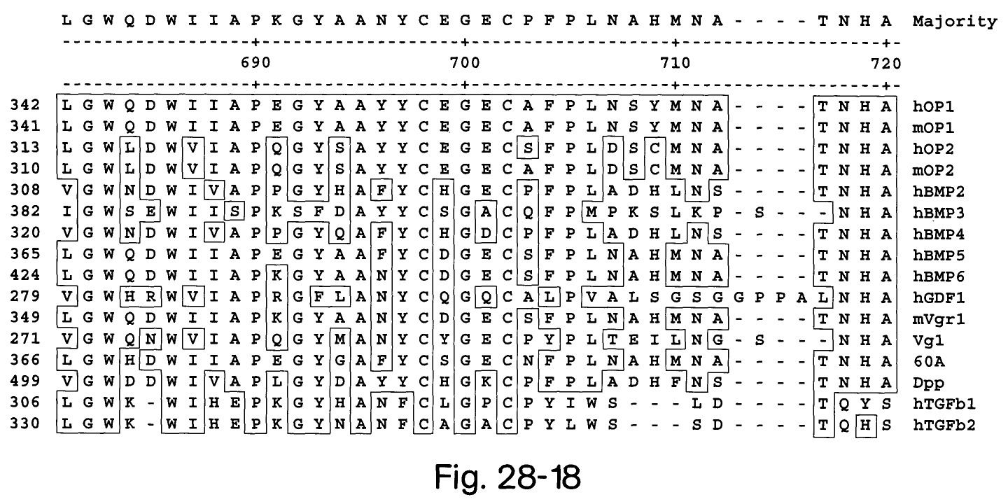

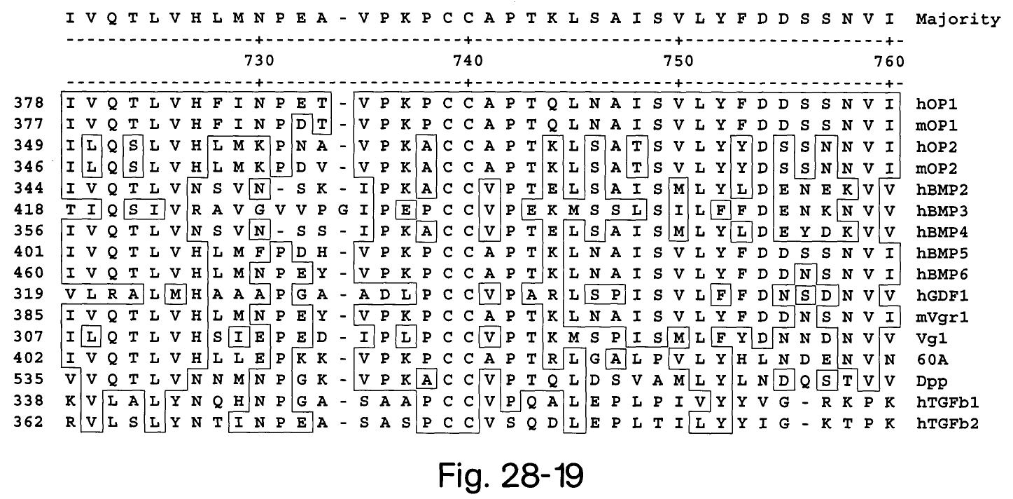

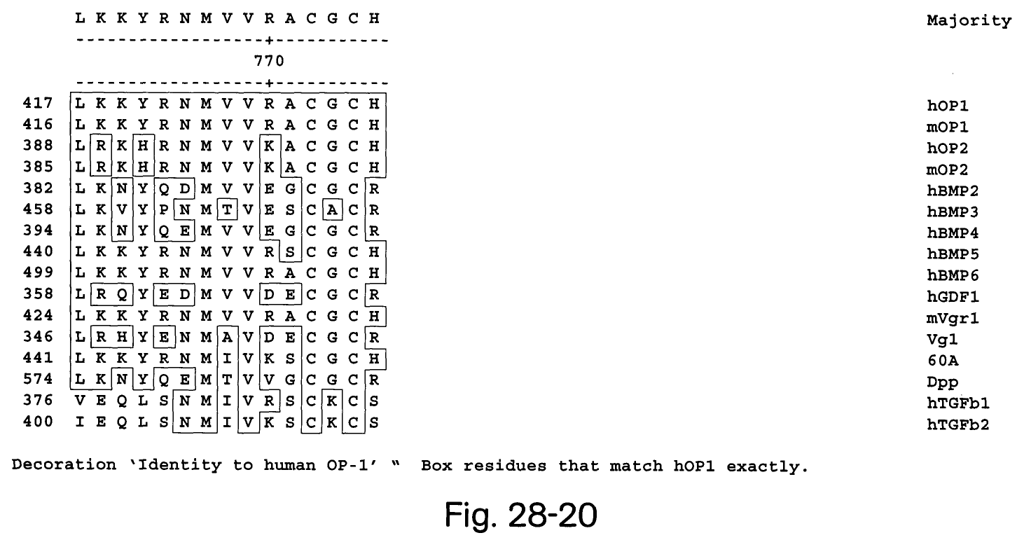

FIG. 28 recites the percent amino acid sequence homology and percent identity within the C-terminal seven cysteine skeletons of various representative members of the TGF-β superfamily, using OP-1 as the reference sequence. The percent homologies recited in the figure are determined by aligning the sequences using the MegaAlign Program (DNAstar, Inc.). Insertions and deletions from the reference morphogen sequence (the C-terminal, biologically active seven-cysteine skeleton of hOP-1) are ignored for purposes of calculation (details see below).

As is apparent to one of ordinary skill in the art reviewing the sequences for the proteins listed in FIG. 28, significant amino acid changes can be made from the reference sequence while retaining substantial morphogenic activity. Moreover, GDF-1 contains a four amino acid insert (Gly-Gly-Pro-Pro, SEQ ID NO: 31) between the two residues corresponding to residue 372 and 373 of OP-1 (SEQ ID NO: 3). Similarly, BMP3 has a “extra” residue, a valine, inserted between the two residues corresponding to residues 385 and 386 of hOP-1 (SEQ ID NO: 3). Also, BMP2 and BMP4 are both “missing” the amino acid residue corresponding to residue 389 of OP-1 (SEQ ID NO: 3). None of these “deviations” from the reference sequence appear to interfere substantially with biological activity.

In other preferred embodiments, the family of morphogenic polypeptides useful in the present invention, and members thereof, are defined by a generic amino acid sequence. For example, Generic Sequence 1 (SEQ ID NO: 24) and Generic Sequence 2 (SEQ ID NO: 25) disclosed below, encompass the observed variations between preferred protein family members identified to date, including at least OP-1, OP-2, OP-3, CBMP2A, CBMP2B, BMP3, 60A, DPP, Vgl, BMP5, BMP6; Vgr-1, and GDF-1. The amino acid sequences for these proteins are described herein and/or in the art, as summarized above. The generic sequences include both the amino acid identity shared by these sequences in the C-terminal skeleton, defined by the six and seven cysteine skeletons (Generic Sequences 1 and 2, respectively), as well as alternative residues for the variable positions within the sequence. The generic sequences provide an appropriate cysteine skeleton where inter- or intra-molecular disulfide bonds can form, and contain certain critical amino acids likely to influence the tertiary structure of the folded proteins. In addition, the generic sequences allow for an additional cysteine at position 36 (Generic Sequence 1) or position 41 (Generic Sequence 2), thereby encompassing the morphogenically active sequences of OP-2 and OP-3.

Generic Sequence 1 (SEQ ID NO: 24)

```
                        Leu Xaa Xaa Xaa Phe Xaa Xaa
                        1 5
Xaa Gly Trp Xaa Xaa Xaa Xaa Xaa Xaa Pro
                10 15
Xaa Xaa Xaa Xaa Ala Xaa Tyr Cys Xaa Gly
                20 25
Xaa Cys Xaa Xaa Pro Xaa Xaa Xaa Xaa Xaa
                30 35
Xaa Xaa Xaa Asn His Ala Xaa Xaa Xaa Xaa
                40 45
Xaa Xaa Xaa Xaa Xaa Xaa Xaa Xaa Xaa Xaa
                50 55
Xaa Xaa Xaa Cys Cys Xaa Pro Xaa Xaa Xaa
                60 65
Xaa Xaa Xaa Xaa Xaa Leu Xaa Xaa Xaa Xaa
                70 75
Xaa Xaa Xaa Val Xaa Leu Xaa Xaa Xaa Xaa
                80 85
Xaa Met Xaa Val Xaa Xaa Cys Xaa Cys Xaa
                90 95
```

wherein each Xaa independently is selected from a group of one or more specified amino acids defined as follows: “Res.” means “residue” and Xaa at res. 2=(Tyr or Lys); Xaa at res. 3=Val or Ile); Xaa at res. 4=(Ser, Asp or Glu); Xaa at res. 6=(Arg, Gln, Ser, Lys or Ala); Xaa at res. 7=(Asp or Glu); Xaa at res. 8=(Leu, Val or Ile); Xaa at res. II=(Gln, Leu, Asp, His, Asn or Ser); Xaa at res. 12=(Asp, Arg, Asn or Glu); Xaa at res. 13=(Trp or Ser); Xaa at res. 14=(Ile or Val); Xaa at res. 15=(Ile or Val); Xaa at res. 16 (Ala or Ser); Xaa at res. 18=(Glu, Gln, Leu, Lys, Pro or Arg); Xaa at res. 19=(Gly or Ser); Xaa at res. 20=(Tyr or Phe); Xaa at res. 21=(Ala, Ser, Asp, Met, His, Gln, Leu or Gly); Xaa at res. 23=(Tyr, Asn or Phe); Xaa at res. 26=(Glu, His, Tyr, Asp, Gln, Ala or Ser); Xaa at res. 28=(Glu, Lys, Asp, Gln or Ala); Xaa at res. 30=(Ala, Ser, Pro, Gln, Ile or Asn); Xaa at res. 31=(Phe, Leu or Tyr); Xaa at res. 33=(Leu, Val or Met); Xaa at res. 34=(Asn, Asp, Ala, Thr or Pro); Xaa at res. 35=(Ser, Asp, Glu, Leu, Ala or Lys); Xaa at res. 36=(Tyr, Cys, His, Ser or Ile); Xaa at res. 37=(Met, Phe, Gly or Leu); Xaa at res. 38=(Asn, Ser or Lys); Xaa at res. 39=(Ala, Ser, Gly or Pro); Xaa at res. 40=(Thr, Leu or Ser); Xaa at res. 44=(Ile, Val or Thr); Xaa at res. 45=(Val, Leu, Met or Ile); Xaa at res. 46=(Gln or Arg); Xaa at res. 47=(Thr, Ala or Ser); Xaa at res. 48=(Leu or Ile); Xaa at res. 49=(Val or Met); Xaa at res. 50=(His, Asn or Arg); Xaa at res. 51=(Phe, Leu, Asn, Ser, Ala or Val); Xaa at res. 52=(Ile, Met, Asn, Ala, Val, Gly or Leu); Xaa at res. 53=(Asn, Lys, Ala, Glu, Gly or Phe); Xaa at res. 54=(Pro, Ser or Val); Xaa at res. 55=(Glu, Asp, Asn, Gly, Val, Pro or Lys); Xaa at res. 56=(Thr, Ala, Val, Lys, Asp, Tyr, Ser, Gly, Ile or His); Xaa at res. 57=(Val, Ala or Ile); Xaa at res. 58=(Pro or Asp); Xaa at res. 59=(Lys, Leu or Glu); Xaa at res. 60=(Pro, Val or Ala); Xaa at res. 63=(Ala or Val); Xaa at res. 65=(Thr, Ala or Glu); Xaa at res. 66=(Gln, Lys, Arg or Glu); Xaa at res. 67=(Leu, Met or Val); Xaa at res. 68=(Asn, Ser, Asp or Gly); Xaa at res. 69=(Ala, Pro or Ser); Xaa at res. 70=(Ile, Thr, Val or Leu); Xaa at res. 71=(Ser, Ala or Pro); Xaa at res. 72=(Val, Leu, Met or Ile); Xaa at res. 74=(Tyr or Phe); Xaa at res. 75=(Phe, Tyr, Leu or His); Xaa at res. 76=(Asp, Asn or Leu); Xaa at res. 77=(Asp, Glu, Asn, Arg or Ser); Xaa at res. 78=(Ser, Gln, Asn, Tyr or Asp); Xaa at res. 79=(Ser, Asn, Asp, Glu or Lys); Xaa at res. 80=(Asn, Thr or Lys); Xaa at res. 82=(Ile, Val or Asn); Xaa at res. 84=(Lys or Arg); Xaa at res. 85=(Lys, Asn, Gln, His, Arg or Val); Xaa at res. 86=(Tyr, Glu or His); Xaa at res. 87=(Arg, Gln, Glu or Pro); Xaa at res. 88=(Asn, Glu, Trp or Asp); Xaa at res. 90=(Val, Thr, Ala or Ile); Xaa at res. 92=(Arg, Lys, Val, Asp, Gln or Glu); Xaa at res. 93=(Ala, Gly, Glu or Ser); Xaa at res. 95=(Gly or Ala) and Xaa at res. 97=(His or Arg).

Generic Sequence 2 (SEQ ID NO: 25) includes all of Generic Sequence I (SEQ ID NO: 24) and in addition includes the following sequence (SEQ ID NO: 26) at its N-terminus:

SEQ ID NO: 26

Cys Xaa Xaa Xaa Xaa
1 5

Accordingly, beginning with residue 7, each “Xaa” in Generic Sequence 2 is a specified amino acid defined as for Generic Sequence 1, with the distinction that each residue number described for Generic Sequence 1 is shifted by five in Generic Sequence 2. Thus, “Xaa. at res. 2=(Tyr or Lys)” in Generic Sequence I refers to Xaa at res. 7 in Generic Sequence 2. In Generic Sequence 2, Xaa at res. 2=(Lys, Arg, Ala or Gln); Xaa at res. 3=(Lys, Arg or Met); Xaa at res. 4=(His, Arg or Gln); and Xaa at res. 5=(Glu, Ser, His, Gly, Arg, Pro, Thr, or Tyr).

In another embodiment, useful osteogenic proteins include those defined by Generic Sequences 3 and 4 (SEQ ID NOs: 27 and 28, respectively), described herein above. Specifically, Generic Sequences 3 and 4 are composite amino acid, sequences of the following proteins: human OP-1, human OP-2, human OP-3, human BMP2, human BMP3, human BMP4, human BMP5, human BMP6, human BMP8, human BMP9, human BMP10, human BMP11, Drosophila 60A, Xenopus Vgl, sea urchin UNIVIN, human CBMP1 (mouse GDF-5), human CBMP2 (mouse GDF-6, human BMP13), human CBMP3 (mouse GDF-7, human BMP12), mouse GDF3, human GDF-1, mouse GDF-1, chicken DORSALIN, Drosophila DPP, Drosophila SCREW, mouse NODAL, mouse GDF-8, human GDF-8, mouse GDF-9, mouse GDF-10, human GDF-11, mouse GDF-11, human BMP15, and rat BMP3b. Like Generic Sequence 1, Generic Sequence 3 accommodates the C-terminal six cysteine skeleton and, like Generic Sequence 2, Generic Sequence 4 accommodates the seven cysteine skeleton.

Generic Sequence 3 (SEQ ID NO: 27)

```
Xaa Xaa Xaa Xaa Xaa Xaa Xaa Xaa Xaa Xaa
1 5 10
Xaa Xaa Xaa Xaa Xaa Xaa Pro Xaa Xaa Xaa
                                15 20
Xaa Xaa Xaa Xaa Cys Xaa Gly Xaa Cys Xaa
                                25 30
Xaa Xaa Xaa Xaa Xaa Xaa Xaa Xaa Xaa Xaa
                                35 40
Xaa Xaa Xaa Xaa Xaa Xaa Xaa Xaa Xaa Xaa
                                45 50
Xaa Xaa Xaa Xaa Xaa Xaa Xaa Xaa Xaa Xaa
                                55 60
Xaa Cys Xaa Pro Xaa Xaa Xaa Xaa Xaa Xaa
                                65 70
Xaa Xaa Leu Xaa Xaa Xaa Xaa Xaa Xaa Xaa
                                75 80
Xaa Xaa Xaa Xaa Xaa Xaa Xaa Xaa Xaa Xaa
                                85 90
Xaa Xaa Xaa Cys Xaa Cys Xaa
                                95
```

wherein each Xaa is independently selected from a group of one or more specified amino acids defined as follows: “Res.” means “residue” and Xaa at res. 1=(Phe, Leu or Glu); Xaa at res. 2=(Tyr, Phe, His, Arg, Thr, Lys, Gln, Val or Glu); Xaa at res 3=(Val, Ile, Leu or Asp); Xaa at res. 4=(Ser, Asp, Glu, Asn or Phe); Xaa at res. 5=(Phe or Glu); Xaa at res. res 6=(Arg, Gln, Lys, Ser, Glu, Ala or Asn); Xaa at res. 7=(Asp, Glu, Leu, Ala or Gln); Xaa at res. 8=(Leu, Val, Met, Ile or Phe); Xaa at res. 9=(Gly, His or Lys); Xaa at res. 10=(Trp or Met); Xaa at res. 11=(Gln, Leu, His, Glu, Asn, Asp, Ser or Gly); Xaa at res. 12=(Asp, Asn, Ser, Lys, Arg, Glu or His); Xaa at res. 13=(Trp or Ser); Xaa at res. 14=(Ile or Val); Xaa at res. 15=(Ile or Val); Xaa at res. 16=(Ala, Ser, Tyr or Trp); Xaa at res. 18=(Glu, Lys, Gln, Met, Pro, Leu, Arg, His or Lys); Xaa at res. 19=(Gly, Glu, Asp, Lys, Ser, Gln, Arg or Phe); Xaa at res. 20=(Tyr or Phe); Xaa at res. 21=(Ala, Ser, Gly, Met, Gln, His, Glu, Asp, Leu, Asn, Lys or Thr); Xaa at res. 22=(Ala or Pro); Xaa at res. 23=(Tyr, Phe, Asn, Ala or Arg); Xaa at res. 24=(Tyr, His, Glu, Phe or Arg); Xaa at res. 26=(Glu, Asp, Ala, Ser, Tyr, His, Lys, Arg, Gln or Gly); Xaa at res. 28=(Glu, Asp, Leu, Val, Lys, Gly, Thr, Ala or Gln); Xaa at res. 30=(Ala, Ser, Ile, Asn, Pro, Glu, Asp, Phe, Gln or Leu); Xaa at res. 31=(Phe, Tyr, Leu, Asn, Gly or Arg); Xaa at res. 32=(Pro, Ser, Ala or Val); Xaa at res. 33=(Leu, Met, Glu, Phe or Val); Xaa at res. 34=(Asn, Asp, Thr, Gly, Ala, Arg, Leu or Pro); Xaa at res. 35=(Ser, Ala, Glu, Asp, Thr, Leu, Lys, Gln or His); Xaa at res. 36=(Tyr, His, Cys, Ile, Arg, Asp, Asn, Lys, Ser, Glu or Gly); Xaa at res. 37=(Met, Leu, Phe, Val, Gly or Tyr); Xaa at res. 38=(Asn, Glu, Thr, Pro, Lys, His, Gly, Met, Val or Arg); Xaa at res. 39=(Ala, Ser, Gly, Pro or Phe); Xaa at res. 40=(Thr, Ser, Leu, Pro, His or Met); Xaa at res. 41=(Asn, Lys, Val, Thr or Gln); Xaa at res. 42=(His, Tyr or Lys); Xaa at res. 43=(Ala, Thr, Leu or Tyr); Xaa at res. 44=(Ile, Thr, Val, Phe, Tyr, Met or Pro); Xaa at res. 45=(Val, Leu, Met, Ile or His); Xaa at res. 46=(Gln, Arg or Thr); Xaa at res. 47=(Thr, Ser, Ala, Asn or His); Xaa at res. 48=(Leu, Asn or Ile); Xaa at res. 49=(Val, Met, Leu, Pro or Ile); Xaa at res. 50=(His, Asn, Arg, Lys, Tyr or Gln); Xaa at res. 51=(Phe, Leu, Ser, Asn, Met, Ala, Arg, Glu, Gly or Gln); Xaa at res. 52=(Ile, Met, Leu, Val, Lys, Gln, Ala or Tyr); Xaa at res. 53=(Asn, Phe, Lys, Glu, Asp, Ala, Gln, Gly, Leu or Val); Xaa at res. 54=(Pro, Asn, Ser, Val or Asp); Xaa at res. 55=(Glu, Asp, Asn, Lys, Arg, Ser, Gly, Thr, Gln, Pro or His); Xaa at res. 56=(Thr, His, Tyr, Ala, Ile, Lys, Asp, Ser, Gly or Arg); Xaa at res. 57=(Val, Ile, Thr, Ala, Leu or Ser); Xaa at res. 58=(Pro, Gly, Ser, Asp or Ala); Xaa at res. 59=(Lys, Leu, Pro, Ala, Ser, Glu, Arg or Gly); Xaa at res.: 60=(Pro, Ala, Val, Thr or Ser); Xaa at res. 61=(Cys, Val or Ser); Xaa at res. 63=(Ala, Val or Thr); Xaa at res. 65=(Thr, Ala, Glu, Val, Gly, Asp or Tyr); Xaa at res. 66=(Gln, Lys, Glu, Arg or Val); Xaa at res. 67=(Leu, Met, Thr or Tyr); Xaa at res. 68=(Asn, Ser, Gly, Thr, Asp, Glu, Lys or Val); Xaa at res. 69=(Ala, Pro, Gly or Ser); Xaa at res. 70=(Ile, Thr, Leu or Val); Xaa at res. 71=(Ser, Pro, Ala, Thr, Asn or Gly); Xaa at res. 2=(Val, Ile, Leu or Met); Xaa at res. 74=(Tyr, Phe, Arg, Thr, Tyr or Met); Xaa at res. 75=(Phe, Tyr, His, Leu, Ile, Lys, Gln or Val); Xaa at res. 76=(Asp, Leu, Asn or Glu); Xaa at res. 77=(Asp, Ser, Arg, Asn, Glu, Ala, Lys, Gly or Pro); Xaa at res. 78=(Ser, Asn, Asp, Tyr, Ala, Gly, Gln, Met, Glu, Asn or Lys); Xaa at res. 79=(Ser, Asn, Glu, Asp, Val, Lys, Gly, Gln or Arg); Xaa at res. 80=(Asn, Lys, Thr, Pro, Val, Ile, Arg; Ser or Gln); Xaa at res. 81=(Val, Ile, Thr or Ala); Xaa at res. 82=(Ile, Asn, Val, Leu, Tyr, Asp or Ala); Xaa at res. 83=(Leu, Tyr, Lys or Ile); Xaa at res. 84=(Lys, Arg, Asn, Tyr, Phe, Thr, Glu or Gly); Xaa at res. 85=(Lys, Arg, His, Gln, Asn, Glu or Val); Xaa at res. 86=(Tyr, His, Glu or Ile); Xaa at res. 87=(Arg, Glu, Gln, Pro or Lys); Xaa at res. 88=(Asn, Asp, Ala, Glu, Gly or Lys); Xaa at res. 89=(Met or Ala); Xaa at res. 90=(Val, Ile, Ala, Thr, Ser or Lys); Xaa at res. 91=(Val or Ala); Xaa at res. 92=(Arg, Lys, Gln, Asp, Glu, Val, Ala, Ser or Thr); Xaa at res. 93=(Ala, Ser, Glu, Gly, Arg or Thr); Xaa at res. 95=(Gly, Ala or Thr); Xaa at res. 97=(His, Arg, Gly, Leu or Ser). Further, after res. 53 in rBMP-3b and mGDF-10 there is an Ile; after res. 54 in GDF-I there is a T; after res. 54 in BMP3 there is a V; after res. 78 in BMP8 and Dorsalin there is a G; after res. 37 in hGDF-1 there is Pro, Gly, Gly, Pro.

Generic Sequence 4 (SEQ ID NO: 28) includes all of Generic Sequence 3 (SEQ ID NO: 27) and in addition includes the following sequence (SEQ ID NO: 26) at its N-terminus:

SEQ ID NO: 26

Cys Xaa Xaa Xaa Xaa
1 5

Accordingly, beginning with residue 6, each “Xaa” in Generic Sequence 4 is a specified amino acid defined as for Generic Sequence 3, with the distinction that each residue number described for Generic Sequence 3 is shifted by five in Generic Sequence 4. Thus, “Xaa at res. 1=(Tyr, Phe, His, Arg, Thr, Lys, Gln, Val or Glu)” in Generic Sequence 3 refers to Xaa at res. 6 in Generic Sequence 4. In Generic Sequence 4, Xaa at res. 2=(Lys, Arg, Gln, Ser, His, Glu, Ala, or Cys); Xaa at res. 3=(Lys, Arg, Met, Lys, Thr, Leu, Tyr, or Ala); Xaa at res. 4=(His, Gln, Arg, Lys, Thr, Leu, Val, Pro, or Tyr); and Xaa at res. 5=(Gln, Thr, His, Arg, Pro, Ser, Ala, Gln, Asn, Tyr, Lys, Asp, or Leu).

Based upon alignment of the naturally occurring morphogens within the definition of Generic Sequence 4, it should be clear that gaps and/or insertions of one or more amino acid residues can be tolerated (without abrogating or substantially impairing biological activity) at least between or involving residues 11-12, 42-43, 59-60, 68-69 and 83-84.

Particularly useful sequences for use as morphogens in this invention include the C-terminal domains, e.g., the C-terminal 96-102 amino acid residues of Vgl, Vgr-1, DPP, OP-1, OP-2, CBMP2A, CBMP2B, GDF-1 (see Table II, below, and SEQ ID NOs: 1-13), as well as proteins comprising the C-terminal domains of 60A, BMP3, BMP5 and BMP6 (see SEQ ID NOs: 12, 14-16), all of which include at least the conserved six or seven cysteine skeleton. In addition, biosynthetic constructs designed from the generic sequences, such as COP-1,3-5, 7, 16, disclosed in U.S. Pat. No. 5,011,691, also are useful. Other sequences include the inhibins/activin proteins (see, for example, U.S. Pat. Nos. 4,968,590 and 5,011,691). Accordingly, other useful sequences are those sharing at least 70% amino acid sequence homology or 50% identity, and preferably 80% homology or 70% identity with any of the sequences above. These are anticipated to include allelic and species variants and mutants, and biosynthetic muteins, as well as novel members of this morphogenic family of proteins. Particularly envisioned in the family of related proteins are those proteins exhibiting morphogenic activity and wherein the amino acid changes from the preferred sequences include conservative changes. Information regarding conserved amino acid changes are well-known in the art. For example, Dayhoff et al. described in Atlas of Protein Sequence and Structure; vol. 5, Suppl. 3, pp. 345-362, (M. O. Dayhoff, ed., Nat'l BioMed. Research Fdn., Washington, D.C. 1978) that certain amino acids substitutions among evolutionary conserved proteins occur at higher than expected frequency than random chance would allow. Thus, conserved amino acid substitutions can be determined according to FIG. 84 (supra). As used herein, potentially useful sequences are aligned with a known morphogen sequence using the method of Needleman et al. ((1970) J. Mol. Biol. 48:443-453) and identities calculated by the MegaAlign program (DNAstar, Inc.).

Table II, set forth below, compares the amino acid sequences of the active regions of native proteins that have been identified as morphogens, including human OP-1 (hOP-1, SEQ ID NOs: 1-3), mouse OP-1 (mOP-1, SEQ ID NOs: 4 and 19), human and mouse OP-2 (SEQ ID NOs: 5, 6, 21, and 23), CBMP2A (SEQ ID NO: 10), CBMP2B (SEQ ID NO: 11), BMP3 (SEQ ID NO: 12), DPP (from Drosophila, SEQ ID NO: 7), Vgl (from Xenopus, SEQ ID NO: 8), Vgr-1 (from mouse, SEQ ID NO: 9), GDF-1 (from mouse, SEQ ID NOs: 13), 60A protein (from Drosophila, SEQ ID NOs: 14), BMP5 (SEQ ID NO: 15) and BMP6 (SEQ ID NO: 16). The sequences are aligned essentially following the method of Needleman et al. (1970) J. Mol. Biol., 48: 443-453, calculated using the Align Program (DNAstar, Inc.) In the table, three dots indicates that the amino acid in that position is the same as the amino acid in hOP-1. Three dashes indicates that no amino acid is present in that position, and are included for purposes of illustrating homologies. For example, amino acid residue 60 of CBMP-2A and CBMP-2B is “missing”. Of course, both these amino acid sequences in this region comprise Asn-Ser (residues 58, 59), with CBMP-2A then comprising Lys and Ile, whereas CBMP-2B comprises Ser and Ile.

TABLE II

| hOP-1 | Cys | Lys | Lys | His | Glu | Leu | Tyr | Val | |
|---|---|---|---|---|---|---|---|---|---|
| mOP-1 | ... | ... | ... | ... | ... | ... | ... | ... | |
| hOP-2 | ... | Arg | Arg | ... | ... | ... | ... | ... | |
| mOP-2 | ... | Arg | Arg | ... | ... | ... | ... | ... | |
| DPP | ... | Arg | Arg | ... | Ser | ... | ... | ... | |
| Vgl | ... | ... | Lys | Arg | His | ... | ... | ... | |
| Vgr-1 | ... | ... | ... | ... | Gly | ... | ... | ... | |
| CBMP-2A | ... | ... | Arg | ... | Pro | ... | ... | ... | |
| CBMP-2B | ... | Arg | Arg | ... | Ser | ... | ... | ... | |
| BMP3 | ... | Ala | Arg | Arg | Tyr | ... | Lys | ... | |
| GDF-1 | ... | Arg | Ala | Arg | Arg | ... | ... | ... | |
| 60A | ... | Gln | Met | Glu | Thr | ... | ... | ... | |
| BMP5 | ... | ... | ... | ... | ... | ... | ... | ... | |
| BMP6 | ... | Arg | ... | ... | ... | ... | ... | ... | |
| | 1 | | | | 5 | | | | |
| hOP-1 | Ser | Phe | Arg | Asp | Leu | Gly | Trp | Gln | Asp |
| mOP-1 | ... | ... | ... | ... | ... | ... | ... | ... | ... |
| hOP-2 | ... | ... | Gln | ... | ... | ... | ... | Leu | ... |
| mOP-2 | Ser | ... | ... | ... | ... | ... | ... | Leu | ... |
| DPP | Asp | ... | Ser | ... | Val | ... | ... | Asp | ... |
| Vgl | Glu | ... | Lys | ... | Val | ... | ... | ... | Asn |
| Vgr-1 | ... | ... | Gln | ... | Val | ... | ... | ... | ... |
| CBMP-2A | Asp | ... | Ser | ... | Val | ... | ... | Asn | ... |
| CBMP-2B | Asp | ... | Ser | ... | Val | ... | ... | Asn | ... |
| BMP3 | Asp | ... | Ala | ... | Ile | ... | ... | Ser | Glu |
| GDF-1 | ... | ... | ... | Glu | Val | ... | ... | His | Arg |
| 60A | Asp | ... | Lys | ... | ... | ... | ... | His | ... |
| BMP5 | ... | ... | ... | ... | ... | ... | ... | ... | ... |
| BMP6 | ... | ... | Gln | ... | ... | ... | ... | ... | ... |
| | | 10 | | | | | 15 | | |
| hOP-1 | Trp | Ile | Ile | Ala | Pro | Glu | Gly | Tyr | Ala |
| mOP-1 | ... | ... | ... | ... | ... | ... | ... | ... | ... |
| hOP-2 | ... | Val | ... | ... | ... | Gln | ... | ... | Ser |
| mOP-2 | ... | Val | ... | ... | ... | Gln | ... | ... | Ser |
| DPP | ... | ... | Val | ... | ... | Leu | ... | ... | Asp |
| Vgl | ... | Val | ... | ... | ... | Gln | ... | ... | Met |
| Vgr-1 | ... | ... | ... | ... | ... | Lys | ... | ... | ... |
| CBMP-2A | ... | ... | Val | ... | ... | Pro | ... | ... | His |
| CBMP-2B | ... | ... | Val | ... | ... | Pro | ... | ... | Gln |
| BMP3 | ... | ... | ... | Ser | ... | Lys | Ser | Phe | Asp |
| GDF-1 | ... | Val | ... | ... | ... | Arg | ... | Phe | Leu |
| 60A | ... | ... | ... | ... | ... | ... | ... | ... | Gly |
| BMP5 | ... | ... | ... | ... | ... | ... | ... | ... | ... |
| BMP6 | ... | ... | ... | ... | ... | Lys | ... | ... | ... |
| | | | 20 | | | | 25 | | |
| hOP-1 | Ala | Tyr | Tyr | Cys | Glu | Gly | Glu | Cys | Ala |
| mOP-1 | ... | ... | ... | ... | ... | ... | ... | ... | ... |
| hOP-2 | ... | ... | ... | ... | ... | ... | ... | ... | Ser |
| mOP-2 | ... | ... | ... | ... | ... | ... | ... | ... | ... |
| DPP | ... | ... | ... | ... | His | ... | Lys | ... | Pro |
| Vgl | ... | Asn | ... | ... | Tyr | ... | ... | ... | Pro |
| Vgr-1 | ... | Asn | ... | ... | Asp | ... | ... | ... | Ser |
| CBMP-2A | ... | Phe | ... | ... | His | ... | Glu | ... | Pro |
| CBMP-2B | ... | Phe | ... | ... | His | ... | Asp | ... | Pro |
| BMP3 | ... | ... | ... | ... | Ser | ... | Ala | ... | Gln |
| GDF-1 | ... | Asn | ... | ... | Gln | ... | Gln | ... | ... |
| 60A | ... | Phe | ... | ... | Ser | ... | ... | ... | Asn |
| BMP5 | ... | Phe | ... | ... | Asp | ... | ... | ... | Ser |
| BMP6 | ... | Asn | ... | ... | Asp | ... | ... | ... | Ser |
| | | | | 30 | | | | | 35 |
| hOP-1 | Phe | Pro | Leu | Asn | Ser | Tyr | Met | Asn | Ala |
| mOP-1 | ... | ... | ... | ... | ... | ... | ... | ... | ... |
| hOP-2 | ... | ... | ... | Asp | ... | Cys | ... | ... | ... |
| mOP-2 | ... | ... | ... | Asp | ... | Cys | ... | ... | ... |
| DPP | ... | ... | ... | Ala | Asp | His | Phe | ... | Ser |
| Vgl | Tyr | ... | ... | Thr | Glu | Ile | Leu | ... | Gly |
| Vgr-1 | ... | ... | ... | ... | Ala | His | ... | ... | ... |
| CBMP-2A | ... | ... | ... | Ala | Asp | His | Leu | ... | Ser |
| CBMP-2B | ... | ... | ... | Ala | Asp | His | Leu | ... | Ser |
| BMP3 | Leu | ... | Val | Ala | Leu | Ser | Gly | Ser† | ... |
| GDF-1 | ... | ... | Met | Pro | Lys | Ser | Leu | Lys | Pro |
| 60A | ... | ... | ... | ... | Ala | His | ... | ... | ... |
| BMP5 | ... | ... | ... | ... | Ala | His | Met | ... | ... |
| BMP6 | ... | ... | ... | ... | Ala | His | Met | ... | ... |
| | | | | | 40 | | | | |
| hOP-1 | Thr | Asn | His | Ala | Ile | Val | Gln | Thr | Leu |
| mOP-1 | ... | ... | ... | ... | ... | ... | ... | ... | ... |
| hOP-2 | ... | ... | ... | ... | ... | Leu | ... | Ser | ... |
| mOP-2 | ... | ... | ... | ... | ... | Leu | ... | Ser | ... |
| DPP | ... | ... | ... | ... | Val | ... | ... | ... | ... |
| Vgl | Ser | ... | ... | ... | ... | Leu | ... | ... | ... |
| Vgr-1 | ... | ... | ... | ... | ... | ... | ... | ... | ... |
| CBMP-2A | ... | ... | ... | ... | ... | ... | ... | ... | ... |
| CBMP-2B | ... | ... | ... | ... | ... | ... | ... | ... | ... |
| BMP3 | Ser | ... | ... | ... | Thr | Ile | ... | Ser | Ile |
| GDF-1 | Leu | ... | ... | ... | Val | Leu | Arg | Ala | ... |
| 60A | ... | ... | ... | ... | ... | ... | ... | ... | ... |
| BMP5 | ... | ... | ... | ... | ... | ... | ... | ... | ... |
| BMP6 | ... | ... | ... | ... | ... | ... | ... | ... | ... |
| | 45 | | | | | 50 | | | |
| hOP-1 | Val | His | Phe | Ile | Asn | Pro | Glu | Thr | Val |
| mOP-1 | ... | ... | ... | ... | ... | ... | ... | ... | ... |
| hOP-2 | ... | His | Leu | Met | Lys | ... | Asn | Ala | ... |
| mOP-2 | ... | His | Leu | Met | Lys | ... | Asp | Val | ... |
| DPP | ... | Asn | Asn | Asn | ... | ... | Gly | Lys | ... |
| Vgl | ... | ... | Ser | ... | Glu | ... | ... | Asp | Ile |
| Vgr-1 | ... | ... | Val | Met | ... | ... | ... | Tyr | ... |
| CBMP-2A | ... | Asn | Ser | Val | ... | Ser | --- | Lys | Ile |
| CBMP-2B | ... | Asn | Ser | Val | ... | Ser | --- | Ser | Ile |
| BMP3 | ... | Arg | Ala† | Gly | Val | Val | Pro | Gly | Ile |
| GDF-1 | Met | ... | Ala | Ala | Ala | ... | Gly | Ala | Ala |
| 60A | ... | ... | Leu | Leu | Glu | ... | Lys | Lys | ... |
| BMP5 | ... | ... | Leu | Met | Phe | ... | Asp | His | ... |
| BMP6 | ... | ... | Leu | Met | ... | ... | ... | Tyr | ... |
| | | 55 | | | | | 60 | | |
| hOP-1 | Pro | Lys | Pro | Cys | Cys | Ala | Pro | Thr | Gln |
| mOP-1 | ... | ... | ... | ... | ... | ... | ... | ... | ... |
| hOP-2 | ... | ... | Ala | ... | ... | ... | ... | ... | Lys |
| mOP-2 | ... | ... | Ala | ... | ... | ... | ... | ... | Lys |
| DPP | ... | ... | Ala | ... | ... | Val | ... | ... | ... |
| Vgl | ... | Leu | ... | ... | ... | Val | ... | ... | Lys |
| Vgr-1 | ... | ... | ... | ... | ... | ... | ... | ... | Lys |
| CBMP-2A | ... | ... | Ala | ... | ... | Val | ... | ... | Glu |
| CBMP-2B | ... | ... | Ala | ... | ... | Val | ... | ... | Glu |
| BMP3 | ... | Glu | ... | ... | ... | Val | ... | Glu | Lys |
| GDF-1 | Asp | Leu | ... | ... | ... | Val | ... | Ala | Arg |
| 60A | ... | ... | ... | ... | ... | ... | ... | ... | Arg |
| BMP5 | ... | ... | ... | ... | ... | ... | ... | ... | Lys |
| BMP6 | ... | ... | ... | ... | ... | ... | ... | ... | Lys |
| | | | 65 | | | | | 70 | |
| hOP-1 | Leu | Asn | Ala | Ile | Ser | Val | Leu | Tyr | Phe |
| mOP-1 | ... | ... | ... | ... | ... | ... | ... | ... | ... |
| hOP-2 | ... | Ser | ... | Thr | ... | ... | ... | ... | Tyr |
| mOP-2 | ... | Ser | ... | Thr | ... | ... | ... | ... | Tyr |
| Vgl | Met | Ser | Pro | ... | ... | Met | ... | Phe | Tyr |
| Vgr-1 | Val | ... | ... | ... | ... | ... | ... | ... | ... |
| DPP | ... | Asn | Ser | Val | Ala | Met | ... | ... | Leu |
| CBMP-2A | ... | Ser | ... | ... | ... | Met | ... | ... | Leu |
| CBMP-2B | ... | Ser | ... | ... | ... | Met | ... | ... | Leu |
| BMP3 | Met | Ser | Ser | Leu | ... | Ile | ... | Phe | Tyr |
| GDF-1 | ... | Ser | Pro | ... | ... | ... | ... | Phe | ... |
| 60A | ... | Gly | ... | Leu | Pro | ... | ... | ... | His |
| BMP5 | ... | ... | ... | ... | ... | ... | ... | ... | ... |
| BMP6 | ... | ... | ... | ... | ... | ... | ... | ... | ... |
| | | | | 75 | | | | | 80 |
| hOP-1 | Asp | Asp | Ser | Ser | Asn | Val | Ile | Leu | Lys |
| mOP-1 | ... | ... | ... | ... | ... | ... | ... | ... | ... |
| hOP-2 | ... | Ser | ... | Asn | ... | ... | ... | ... | Arg |
| mOP-2 | ... | Ser | ... | Asn | ... | ... | ... | ... | Arg |
| DPP | Asn | ... | Gln | ... | Thr | ... | Val | ... | ... |
| Vgl | ... | Asn | Asn | Asp | ... | ... | Val | ... | Arg |
| Vgr-1 | ... | ... | Asn | ... | ... | ... | ... | ... | ... |
| CBMP-2A | ... | Glu | Asn | Glu | Lys | ... | Val | ... | ... |
| CBMP-2B | ... | Glu | Tyr | Asp | Lys | ... | Val | ... | ... |
| BMP3 | ... | Glu | Asn | Lys | ... | ... | Val | ... | ... |
| GDF-1 | ... | Asn | ... | Asp | ... | ... | Val | ... | Arg |
| 60A | Leu | Asn | Asp | Glu | ... | ... | Asn | ... | ... |
| BMP5 | ... | ... | ... | ... | ... | ... | ... | ... | ... |
| BMP6 | ... | ... | Asn | ... | ... | ... | ... | ... | ... |
| | | | | | 85 | | | | |
| hOP-1 | Lys | Tyr | Arg | Asn | Met | Val | Val | Arg | |
| mOP-1 | ... | ... | ... | ... | ... | ... | ... | ... | |
| hOP-2 | ... | His | ... | ... | ... | ... | ... | Lys | |
| mOP-2 | ... | His | ... | ... | ... | ... | ... | Lys | |
| DPP | Asn | ... | Gln | Glu | ... | Thr | ... | Val | |
| Vgl | His | ... | Glu | ... | ... | Ala | ... | Asp | |
| Vgr-1 | ... | ... | ... | ... | ... | ... | ... | ... | |
| CBMP-2A | Asn | ... | Gln | Asp | ... | ... | ... | Glu | |
| CBMP-2B | Asn | ... | Gln | Glu | ... | ... | ... | Glu | |
| BMP3 | Val | ... | Pro | ... | ... | Thr | ... | Glu | |
| GDF-1 | Gln | ... | Glu | Asp | ... | ... | ... | Asp | |
| 60A | ... | ... | ... | ... | ... | Ile | ... | Lys | |
| BMP5 | ... | ... | ... | ... | ... | ... | ... | ... | |
| BMP6 | ... | ... | ... | Trp | ... | ... | ... | ... | |
| | 90 | | | | | | 95 | | |
| hOP-1 | Ala | Cys | Gly | Cys | His | | | | |
| mOP-1 | ... | ... | ... | ... | ... | | | | |
| hOP-2 | ... | ... | ... | ... | ... | | | | |
| mOP-2 | ... | ... | ... | ... | ... | | | | |
| DPP | Gly | ... | ... | ... | Arg | | | | |
| Vgl | Glu | ... | ... | ... | Arg | | | | |
| Vgr-1 | ... | ... | ... | ... | ... | | | | |
| CBMP-2A | Gly | ... | ... | ... | Arg | | | | |
| CBMP-2B | Gly | ... | ... | ... | Arg | | | | |
| BMP3 | Ser | ... | Ala | ... | Arg | | | | |
| GDF-1 | Glu | ... | ... | ... | Arg | | | | |
| 60A | Ser | ... | ... | ... | ... | | | | |
| BMP5 | Ser | ... | ... | ... | ... | | | | |
| BMP6 | ... | ... | ... | ... | ... | | | | |
| | | | 100 | | | | | | |

†Between residues 56 and 57 of BMP3 is a Val residue; between residues 43 and 44 of GDF-1 lies the amino acid sequence Gly-Gly-Pro-Pro.

As is apparent from the foregoing amino acid sequence comparisons, significant amino acid changes can be made within the generic sequences while retaining the morphogenic activity.

As noted above, certain preferred morphogenic polypeptide sequences useful in this invention have greater than 50% identity, preferably greater than 55%, 60%, 65%, 70%, 80%, 85%, 90%, 95% or even 99% identity, with the amino acid sequence defining the preferred reference sequence of hOP-1 (especially the conserved six-seven cysteine skeleton of hOP-1, e.g., residues 39-139 of SEQeq ID No: 1), or equivalent regions from other morphogens described in the application. These particularly preferred sequences include allelic and phylogenetic counterpart variants of the OP-1 and OP-2 proteins, including the Drosophila 60A protein, as well as the closely related proteins BMP5, BMP6 and Vgr-1. Accordingly, in certain particularly preferred embodiments, useful morphogenic proteins include active proteins comprising pairs of polypeptide chains within the generic amino acid sequence herein referred to as “OPX” (SEQ ID NO: 29), which defines the seven cysteine skeleton and accommodates the homologies between several identified variants of OP-1 and OP-2. Accordingly, each “Xaa” at a given position in OPX independently is selected from the residues occurring at the corresponding position in the C-terminal sequence of mouse or human OP-1 or OP-2. Specifically, each “Xaa” is independently selected from a group of one or more specified amino acids as defined below:

```
Cys Xaa Xaa His Glu Leu Tyr Val Ser Phe Xaa Asp Leu Gly Trp
  1 5 10 15
Xaa Asp Trp Xaa Ile Ala Pro Xaa Gly Tyr Xaa Ala Tyr Tyr Cys
                                20 25 30
Glu Gly Glu Cys Xaa Phe Pro Leu Xaa Ser Xaa Met Asn Ala Thr
                                35 40 45
Asn His Ala Ile Xaa Gln Xaa Leu Val His Xaa Xaa Xaa Pro Xaa
                                50 55 60
Xaa Val Pro Lys Xaa Cys Cys Ala Pro Thr Xaa Leu Xaa Ala Xaa
                                65 70 75
Ser Val Leu Tyr Xaa Asp Xaa Ser Xaa Asn Val Ile Leu Xaa Lys
                                80 85 90
Xaa Arg Asn Met Val Xaa Ala Cys Gly Cys His
                                95 100
```

wherein Xaa at res. 2=(Lys or Arg); Xaa at res. 3=(Lys or Arg); Xaa at res. 11=(Arg or Gln); Xaa at res. 16=(Gin or Leu); Xaa. at res. 19=(Ile or Val); Xaa at res. 23=(Glu or Gln); Xaa at res. 26=(Ala or Ser); Xaa at res. 35=(Ala or Ser); Xaa at res. 39=Asn or Asp); Xaa at res. 41=(Tyr or Cys); Xaa at res. 50=(Val or Leu); Xaa at res. 58=(Asn or Thr); Xaa at res. 56=(Phe or Leu); Xaa at res. 57=(Ile or Met); Xaa at res. 58=(Asn or Lys); Xaa at res. 60=(Glu, Asp or Asn); Xaa at res. 61=(Thr, Ala or Val); Xaa at res. 65=(Pro or Ala); Xaa at res. 71=(Gln or Lys): Xaa at res. 73=(Asn or Ser); Xaa at res. 75=(Ile or Thr); Xaa at res. 80=(Phe or Tyr); Xaa at res. 82=(Asp or Ser); Xaa at res. 84=(Ser or Asn); Xaa at res. 89=(Lys or Arg); Xaa at res. 91=(Tyr or His); and Xaa at res. 97=(Arg or Lys).

The following patents or publications or patent applications disclose morphogens or formula of useful/active morphogens, the entire contents of which are hereby incorporated by reference herein: EP 601106, and U.S. Ser. No. 08/937,755 (filed on Sep. 25, 1997).

The morphogens useful in the methods, composition and devices of this invention include proteins comprising any of the polypeptide chains described above, whether isolated from naturally occurring sources, or produced by recombinant DNA or other synthetic techniques, and includes allelic and species variants of these proteins, naturally occurring or biosynthetic mutants thereof, as well as various truncated and fusion constructs. Deletion or addition mutants also are envisioned to be active, including those which may alter the conserved C-terminal cysteine skeleton, provided that the alteration does not functionally disrupt the relationship of these cysteines in the folded structure. Accordingly, such active forms are considered the equivalent of the specifically described constructs disclosed herein.

The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The morphogenic proteins can be expressed from intact or truncated cDNA or from synthetic DNAs in prokaryotic or eukaryotic host cells, and purified, cleaved, refolded, and dimerized to form morphogenically active compositions. Currently preferred host cells include E. Coli or any suitable mammalian host cells, such as CHO, COS or BSC cells. A detailed description of the morphogens useful in the methods, compositions and devices of this invention is disclosed in copending U.S. patent application Ser. Nos. 08/937,755, filed Sep. 25, 1997, and issued European Patent EP 601106, the contents of which are all incorporated by reference herein. Thus, in view of this disclosure, skilled genetic engineers can isolate genes from cDNA or genomic libraries of various different species which encode appropriate amino acid sequences, or construct DNAs from oligonucleotides, and then can express them in various types of host cells, including both prokaryotes and eukaryotes, to produce large quantities of active proteins capable of protecting tissues and organs from immune cell-mediated tissue destruction, including substantially inhibiting such damage and/or regenerating the damaged tissue in a variety of mammals, including humans.

In still another preferred embodiment, useful morphogenically active proteins have polypeptide chains with amino acid sequences comprising a sequence encoded by a nucleic acid that hybridizes with DNA or RNA encoding reference morphogen sequences, e.g., C-terminal sequences defining the conserved seven cysteine skeletons of OP-1, OP-2, BMP2, BMP4, BMP5, BMP6, 60A, GDF-3, GDF-5, GDF-6, GDF-7 and the like. As used herein, high stringency hybridization conditions are defined as hybridization according to known techniques in 40% formamide, 5×SSPE, 5× Denhardt's Solution, and 0.1% SDS at 37° C. overnight, and washing in 0.1×SSPE, 0.1% SDS at 50° C. Standard stringency conditions are well characterized in standard molecular biology cloning texts. See, for example, MOLECULAR CLONING—A LABORATORY MANUAL, 2nd Ed., ed. by Sambrook, Fritsch and Maniatis (Cold Spring Harbor Laboratory Press: 1989); DNA CLONING, Volumes I and II (D. N. Glover ed., 1985); OLIGONUCLEOTIDE SYNTHESIS (M. J. Gait ed., 1984); NUCLEIC ACID HYBRIDIZATION (B. D. Hames & S. J. Higgins eds. 1984); and B. Perbal, A PRACTICAL GUIDE TO MOLECULAR CLONING (1984).

In other embodiments, as an alternative to the administration of a morphogenic protein, an effective amount of an agent competent to stimulate or induce increased endogenous morphogen expression in a mammal may be administered by any of the routes described herein. Such a morphogen inducer may be provided to a mammal, e.g., by systemic administration to the mammal or by direct administration to the neural tissue. A method for identifying and testing inducers (stimulating agents) competent to modulate the levels of endogenous morphogens in a given tissue is described in published applications WO 93/05172 and WO 93/05751, each of which is incorporated by reference herein. Briefly, candidate compounds are identified and tested by incubation in vitro with test tissue or cells, or a cultured cell line derived therefrom, for a time sufficient to allow the compound to affect the production, i.e., cause the expression and/or secretion, of a morphogen produced by the cells of that tissue. Suitable tissues, or cultured cells of a suitable tissue, are preferably selected from renal epithelium, ovarian tissue, fibroblasts, and osteoblasts.

In yet other embodiments, an agent which acts as an agonist of a morphogen receptor may be administered instead of the morphogen itself. Such an agent may also be referred to as a morphogen “mimic,” “mimetic,” or “analog.” Thus, for example, a small peptide or other molecule which can mimic the activity of a morphogen in binding to and activating the morphogen's receptor—may be employed as an equivalent of the morphogen. Preferably the agonist is a full agonist, but partial morphogen receptor agonists may also be advantageously employed. Methods of identifying such agonists are known in the art and include assays for compounds which induce morphogen-mediated responses (e.g., induction of differentiation of metanephric mesenchyme, induction of endochondral bone formation). For example, methods of identifying morphogen inducers or agonists of morphogen receptors may be found in U.S. Ser. No. 08/478,097 filed Jun. 7, 1995; U.S. Ser. No. 09/791,946, filed Feb. 22, 2001; U.S. Pat. No. 5,834,188; U.S. Pat. No. 6,273,598; WO 97/26277; EP 0876401; U.S. Provisional Application No. 60/080,032, filed on Mar. 30, 1998; U.S. Provisional Application No. 60/296,291, filed on Jun. 10, 2001; U.S. Provisional Application No. 60/354,820, filed on Feb. 5, 2002; and U.S. Provisional Application filed on Apr. 10, 2002 (first named inventor Peter Keck, title: “MORPHOGEN ANALOGS AND METHODS FOR PRODUCING THEM”), the disclosures of which are incorporated herein by reference.

The OP/BMP family of morphogens of the invention are also characterized by biological activities which may be readily ascertained by those of ordinary skill in the art. Specifically, a morphogen of the present invention is (a) capable of inducing chondrogenesis in the Reddi-Sampath ectopic bone assay (Sampath and Reddi (1981), Proc. Natl. Acad. Sci. USA 78:7599-7603) or a substantially equivalent assay, (b) capable of significantly preventing, inhibiting, delaying or alleviating the progressive loss of renal function in a standard animal model of chronic renal failure, or (c) capable of causing a clinically significant improvement in a standard marker of renal function when administered to a mammal in, or at risk of, chronic renal failure.

The Reddi-Sampath ectopic bone assay is well known in the art as an assay of chondrogenic activity. The assay, which can be easily performed, is described and discussed in, for example, Sarnpath and Reddi (1981), Proc. Natl. Acad. Sci. USA 78: 7599-7603; and function which characterizes chronic renal failure.

Finally, the morphogens of the present invention may be evaluated for their therapeutic efficacy in causing a clinically significant improvement in a standard marker of renal function when administered to a mammalian subject (e.g., a human patient) in, or at risk of, chronic renal failure. Such markers of renal function are well known in the medical literature and include, without being limited to, rates of increase in BUN levels, rates of increase in serum creatinine, static measurements of BUN, static measurements of serum creatinine, glomerular filtration rates (GFR), ratios of BUN/creatinine, serum concentrations of sodium (Na⁺), urine/plasma ratios for creatinine, urine/plasma ratios for urea, urine osmolality, daily urine output, and the like (see, for example, Brenner and Lazarus (1994), in Harrison's Principles of Internal Medicine, 13th edition, Isselbacher et al., eds., McGraw Hill Text, New York; Luke and Strom (1994), in Internal Medicine, 4th Edition, J. H. Stein, ed., Mosby-Year Book, Inc. St. Louis).

The morphogens contemplated herein can be expressed from intact or truncated genomic or cDNA or from synthetic DNAs in prokaryotic or eukaryotic host cells. The dimeric proteins can be isolated from the culture media and/or refolded and dimerized in vitro to form biologically active compositions, Heterodimers can be formed in vitro by combining separate, distinct polypeptide chains. Alternatively, heterodimers can be formed in a single cell by co-expressing nucleic acids encoding separate, distinct polypeptide chains. See, for example, WO93/09229, or U.S. Pat. No. 5,411,941, for several exemplary recombinant heterodimer protein production protocols. Currently preferred host cells include, without limitation, prokaryotes including E. coli, or eukaryotes including yeast (such as S. cerevisiae), insect cells, or any suitable mammalian host cells, such as CHO, COS or BSC cells. One of ordinary skill will appreciate that other host cells can be used to advantage. A detailed description of the morphogens useful in the methods, compositions and devices of this invention, including how to make, use and test them for chondrogenic activity, are disclosed in numerous publications, including U.S. Pat. Nos. 5,266,683 and 5,011,691, the specifications of which are incorporated herein by reference.

As a general matter, methods of the present invention may be applied to the treatment of any mammalian subject at risk of or afflicted with a neural tissue insult or neuropathy. The invention is suitable for the treatment of any primate, preferably a higher primate such as a human. In addition, however, the invention may be employed in the treatment of domesticated mammals which are maintained as human companions (e.g., dogs, cats, horses), which have significant commercial value (e.g., goats, pigs, sheep, cattle, sporting or draft animals), which have significant scientific value (e.g., captive or free specimens of endangered species, or inbred or engineered animal strains), or which otherwise have value.

(ii) Inhibitors of ACE (Angiotensin-Converting Enzyme)

Angiotensin I-converting enzyme (EC 3.4.15.1), or kininase II, is a dipeptidyl carboxypeptidase that plays an important role in blood pressure regulation and electrolyte balance by hydrolyzing angiotensin I into angiotensin II, a potent vasopressor, and aldosterone-stimulating peptide. The enzyme is also able to inactivate bradykinin, a potent vasodilator. The ACE gene encodes 2 isozymes. The somatic ACE isozyme is expressed in many tissues, including vascular endothelial cells, renal epithelial cells, and testicular Leydig cells, whereas the testicular or germinal ACE isozyme is expressed only in sperm (Ramaraj et al., J. Clin. Invest. 102: 371-378, 1998).

The angiotensin converting enzyme (ACE) inhibitors of the present invention may include 3-amino-[1]benzazepin-2-one-1-alkanoic acids and derivatives, as disclosed in U.S. Pat. Nos. 4,473,575 and 4,410,520 (the entire contents of which are incorporated herein by reference), and characterized by formula (I) shown below: 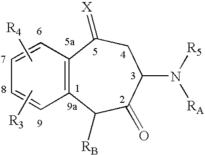
wherein R_{A} and R_{B} are radicals of the formula: 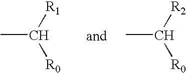
respectively, in which:
Rₒ is carboxy or a functionally modified carboxy;
R₁ is hydrogen, lower alkyl, amino(lower)alkyl, aryl, aryl(lower)alkyl, cycloalkyl, cycloalkyl(lower)alkyl, acylamino(lower) alkyl, mono- or di-(lower)alkylamino(lower)alkyl, lower alkylthio(lower)alkyl, carboxy(lower)alkyl, esterified carboxy(lower)alkyl, carbamoyl(lower)alkyl, etherified or acylated hydroxyl(lower)alkyl, aryloxy(-lower)alkyl, aryl-(thio-, sulfinyl-, or sulfonyl-)lower alkyl, aryl-N-(lower)alkylamino(lower)alkyl, or arylamino(lower)alkyl;
R₂ is hydrogen or lower alkyl;
R₃ and R₄, each independently, represent hydrogen, lower alkyl, lower alkoxy, lower alkanoyloxy, hydroxyl, halogen, trifluoromethyl, or R₃ and R₄ taken together represent lower alkylenedioxy;
R₅ is hydrogen or lower alkyl, and
X represents oxo, two hydrogens, or one hydroxyl or acylated hydroxy together with one hydrogen; and wherein the carbocyclic ring may also be hexahydro or 6,7,8,9-tetrahydro; and salts and complexes thereof.

The functionally modified carboxyl group in the meaning of the symbol Rₒ may be, for example, an esterified carboxyl group or a carbamoyl group optionally substituted on the nitrogen atom. More specifically one or both of Ro represented by COR₆ in radical R_{A} and represented by COR₇ in radical RB may independently represent carboxy, esterified carboxy, carbamoyl or substituted carbamoyl.

ACE inhibitors of the present invention may also include bicyclic compounds and their derivatives disclosed in U.S. Pat. No. 4,385,051 (the entire contents of which are incorporated herein by reference), and represented by formula (II) shown below: 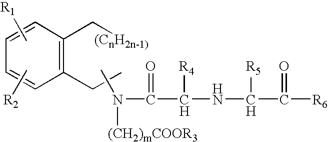
wherein R₁ and R₂ are the same or different and each represents hydrogen, hydroxyl or lower alkoxy;
R₃ is hydrogen or lower alkyl;
R₄ is hydrogen, lower alkyl, amino-lower-alkyl or acylamino-lower-alkyl;
R₅ is hydrogen, lower alkyl or aralkyl which may be substituted;
R₆ is hydroxyl, lower alkoxy, amino or lower alkylamino;
and m and n each means 1 or 2, and salts thereof.

ACE inhibitors of the present invention may also include phosphinylalkanoyl substituted proline compounds disclosed in U.S. Pat. No. 4,337,201 (the entire contents of which are incorporated herein by reference), and represented by formula (III) shown below: 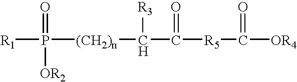
and salts thereof, wherein R₁ is alkyl, aryl, arylalkyl, cycloalkyl, or cycloalkyl(alkyl);
R₂ and R₄ each is independently hydrogen, alkyl, arylalkyl or 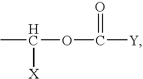
   wherein X is hydrogen, alkyl, or phenyl and Y is hydrogen, alkyl, phenyl or alkoxy, or together X and Y are —(CH₂)₂—, —(CH₂)₃—, —CH═CH—, or 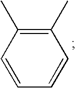
   R₃ is hydrogen or alkyl;
   —R₅—COOR₄ is 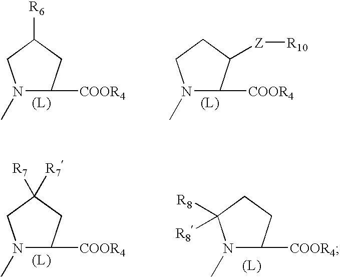
   R₆ is hydrogen, hydroxyl, alkyl, halogen, azido, amino, cycloalkyl, aryl, arylalkyl, carbamoyloxy; 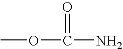
   N,N-dialkylcarbamoyloxy, or -Z-R₉;
   R₇ and R₇ are the same and each is halogen or -Z-R₁₀, or R₇ and R₇ together are ═O, —O—(CH₂—)ₘ—O— or —S—(CH₂)ₘ—S—;
   R₈ is hydrogen and R₈ is phenyl, 2 hydroxyphenyl or 4-hydroxyphenyl or R₈ and R₈ together are ═O;
   R₉ is alkyl, aryl, arylalkyl, 1- or 2-napthyl, or biphenyl;
   R₁₀ is alkyl, aryl or arylalkyl;
   Z is oxygen or sulfur;
   n is 0 or 1; and
   m is 1 or 2; with the proviso that if —R₅—COOR₄ is 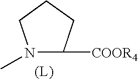
   at least one of R₂ and R₄ is

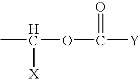

ACE inhibitors of the present invention may also include azetidine-2-carboxylic acid derivative compounds disclosed in U.S. Pat. No. 4,046,889 (the entire contents of which are incorporated herein by reference), and represented by formula (IV) shown below: 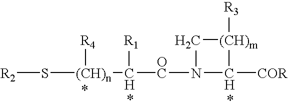
wherein R is hydroxy, NH₂ or lower alkoxy;
R₁ and R₄ each is hydrogen, lower alkyl or phenyl-lower alkyl;
R₂ is hydrogen or R₅—CO;
R₃ is hydrogen, hydroxy or lower alkyl;
R₅ is lower alkyl, phenyl or phenyl-lower alkyl;
m is 1 to 3;
n is 0 to 2, and the asterisks indicate asymmetric carbon atoms. The carbon in the acyclic side chain is asymmetric when R₁ is other than hydrogen.

ACE inhibitors of the present invention may also include carboxyalkyl dipeptide compounds and derivatives thereof, disclosed in U.S. Pat. No. 4,374,829 (the entire contents of which are incorporated herein by reference), and represented by formula (V) shown below: 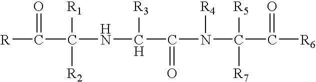
wherein R and R₆ are the same or different and are hydroxy, lower alkoxy, lower alkenoxy, dilower alkylamino lower alkoxy (dimethylaminoethoxy), acylamino lower alkoxy (acetylaminoethoxy), acyloxy lower alkoxy (pivaloyloxymethoxy), aryloxy such as phenoxy, aryl(lower)alkoxy such as benzyloxy; substituted aryloxy or substituted aryl(lower)alkoxy wherein the substituent is methyl, halo or methoxy, amino, lower alkylamino, di(lower)alkylamino, hydroxyamino, or aryl(lower)alkylamino such as benzylamino;
R₁ is hydrogen, alkyl of from 1 to 20 carbon atoms which include branched and cyclic and unsaturated (such as allyl) alkyl groups, substituted lower alkyl wherein the substituent can be halo, hydroxy, lower alkoxy, aryloxy such as phenoxy, amino, dilower alkylamino, acylamino, such as acetamido and benzamidoarylamino, guanidino, imidazolyl, indolyl, mercapto, lower alkylthio, arylthio such as phenylthio, carboxy or carboxyamido, carbolower alkoxy, aryl such as phenyl or naphthyl, substituted aryl such as phenyl wherein the substituent is lower alkyl, lower alkoxy or halo, aryl lower alkyl, aryl lower alkenyl, heteroaryl lower alkyl or heteroaryl lower alkenyl such as benzyl, styryl or indolyl ethyl, substituted aryl lower alkyl, substituted aryl lower alkenyl, substituted heteroaryl lower alkyl, or substituted heteroaryl lower alkenyl, wherein the substituent(s) is halo, dihalo, lower alkyl, hydroxy, lower alkoxy, amino, aminomethyl, acylamino (acetylamino or benzoylamino) dilower alkylamino, lower alkylamino, carboxyl, halolower alkyl, cyano or sulfonamide, aryl lower alkyl or heteroaryllowrealkyl substituted on the alkyl portion by amino or acylamino (acetylamino or benzoylamino);
R₂ and R₇ are the same or different and are hydrogen or lower alkyl;
R₃ is hydrogen, lower alkyl, phenyl lower alkyl, aminomethyl phenyl lower alkyl, hydroxy phenyl lower alkyl, hydroxy lower alkyl, acylamino lower alkyl (such as benzoylamino lower alkyl, acetylamino lower alkyl) amino lower alkyl, dimethylamino lower alkyl, halo lower alkyl, guanidino lower alkyl, imidazolyl lower alkyl, indolyl lower alkyl, mercapto lower alkyl, lower alkyl thio lower alkyl;
R₄ is hydrogen or lower alkyl;
R₅ is hydrogen, lower alkyl, phenyl, phenyl lower alkyl, hydroxylphenyl lower alkyl, hydroxyl lower alkyl, amino lower alkyl, guanidino lower alkyl, imidazolyl lower alkyl, indolyl lower alkyl, imidazolyl lower alkyl, indolyl lower alkyl, mercapto lower alkyl or lower alkyl thio lower alkyl;
R₄ and R₅ may be connected together to form an alkylene bridge of from 2 to 4 carbon atoms, an alkylene bridge of form 2 to 3 carbon atoms and one sulfur atom, an alkylene bridge of from 3 to 4 carbon atoms containing a double bond or an alkylene bridge as above substituted with hydroxyl, lower alkoxy, lower alkyl or dilower alkyl.

ACE inhibitors of the present invention may also include substituted iminodiacid compounds as disclosed in U.S. Pat. No. 4,508,729 (the entire contents of which are incorporated herein by reference), and represented by formula (VI) shown below: 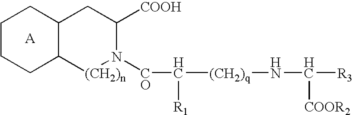
wherein the ring A is saturated and n=0 or 1, or the ring A is a benzene ring and n=1,
R₁ represents a lower alkyl group having from 1 to 4 carbon atoms which can carry an amino group,
R₂ represents a hydrogen atom or an alkyl group having from 1 to 4 carbon atoms,
R₃ represents a straight or branched alkyl group, a mono- or dicycloalkylalkyl or phenylalkyl group having no more than a total of 9 carbon atoms, or a substituted alkyl group of the formula: 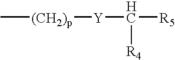
with R₄=H, a lower alkyl (C₁ to C₄) or a cycloalkyl (C₃ to C₆) group,
R₅═H, a lower alkyl (C₁ to C₄), a cycloalkyl (C₃ to C₆) or an alkoxycarbonyl group,
Y═S or >N-Q where Q=H, or an acetyl or benzyloxycarbonyl group, and p=1 or 2, and q=0 or 1.

The ACE inhibitor compounds of the present invention may also include substituted acyl derivatives of 1,2,3,4-tetrahydroisoquinoline-3-carboxylic acid compounds disclosed in U.S. Pat. No. 4,344,949 (the entire contents of which are incorporated herein by reference), and having formula (VII) as shown below: 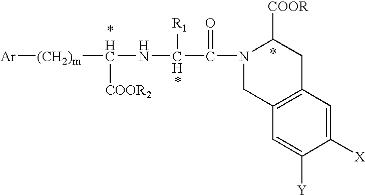
where R is hydrogen, lower alkyl or aralkyl;
R₁ is hydrogen, lower alkyl, or benzyl;
R₂ is hydrogen or lower alkyl, and Ar is phenyl or phenyl substituted with 1 or 2 substituents selected from the group consisting of fluorine, chlorine, bromine, lower alkyl, lower alkoxy, hydroxy or amino;
X and Y are independently hydrogen, lower alkyl, lower alkoxy, lower alkylthio, lower alkylsufinyl, lower alkylsulfonyl, hydroxy, or X and Y together are methylenedioxy;
m is 0 to 3;
and pharmaceutically acceptable salts thereof.

The ACE inhibitors may also include phosphinylalkanoyl proline compounds disclosed in U.S. Pat. No. 4,168,267 (the entire content of which is incorporated herein by reference), which have formula (VIII) as shown below: 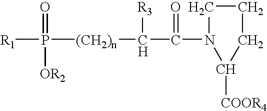
wherein R₁ is lower alkyl, phenyl or phenyl-lower alkyl;
R₂ is hydrogen, phenyl-lower alkyl or a metal ion;
R₃ is hydrogen or lower alkyl;
R₄ is hydrogen, lower alkyl, phenyl-lower alkyl or a metal ion; and
n is 0 or 1.

The ACE inhibitor compounds of the present invention may also include ether and thioether mercaptoacyl proline compounds disclosed in U.S. Pat. No. 4,316,906 (the entire content of which is incorporated herein by reference), and having formula (IX) as shown below: 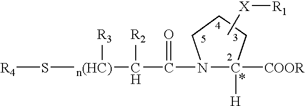
wherein the group X—R₁ is located at the 3- or 4-position in the ring;
X is oxygen or sulfur;
R is hydrogen or lower alkyl;
R₁ is lower alkyl, lower alkenyl, lower alkynyl, cycloalkyl, 1- or 2-adamantyl, aryl, substituted aryl, phenyl-lower alkylene or substituted phenyl-lower alkylene.
R₂ and R₃ are independently selected from hydrogen, lower alkyl, and trifluoromethyl;
R₄ is hydrogen, R₅—CO—, or 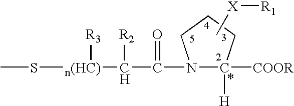
R₅ is lower alkyl, phenyl, phenyl-lower alkylene; substituted phenyl, or substituted phenyl-lower alkylene;
n is 0, 1 or 2; and salts thereof.

The ACE inhibitor compounds of the present invention also may include proline derivatives and related compounds disclosed in U.S. Pat. No. 4,105,776 (the entire contents of which are incorporated herein by reference), which have the general formula (X) as shown below: 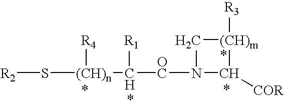
wherein R is hydroxy, NH₂ or lower alkoxy;
R₁ and R₄ each is hydrogen, lower alkyl, phenyl or phenyl-lower alkyl;
R₂ is hydrogen, lower alkyl, phenyl, substituted phenyl wherein the phenyl substituent is halo, lower alkyl or lower alkoxy, phenyl-lower alkyl, diphenyl-lower alkyl, triphenyl-lower alkyl, lower alkylthiomethyl, phenyl-lower alkythiomethyl, lower alkanoyl-amidomethyl, 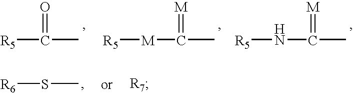
R₃ is hydrogen, hydroxyl or lower alkyl;
R₅ is lower alkyl, phenyl or phenyl-lower alkyl;
R₆ is lower alkyl, phenyl, substituted phenyl, (wherein the phenyl substituent is halo, lower alkyl or lower alkoxy), hydroxyl-lower alkyl or amino(carboxy)lower alkyl; 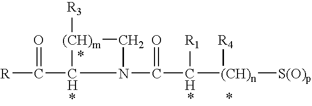
M is O or S;
m is 1 to 3; and
n and p each is 0 to 2.

The asterisks indicate asymmetric carbon atoms. Each of the carbons bearing a substituent R₁, R₃ and R₄ is asymmetric when that substituent is other than hydrogen.

The ACE inhibitors of the present invention may also include bicyclic pyridazo[1,2-A][1,2] diazepine compounds disclosed in U.S. Pat. No. 4,512,924 (the entire contents of which are incorporated herein by reference), having the general formula (XI) as shown below: 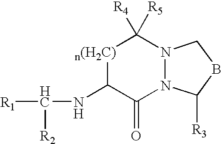
wherein B represent a methylene (—CH₂—), ethylene (—CH₂—CH₂—) or vinylene (—CH═CH—) group;
R₁ represents a hydrogen atom or an alkyl, aralkyl, amino-alkyl, monoalkylamino-alkyl, dialkylaminoalkyl, acylamino-alkyl, phthalimido-alkyl, alkoxycarbonylamino-alkyl, aryloxycarbonylamino-alkyl, aralkoxycarbonlyamino-alkyl, alkylaminocarbonylaminoalkyl, arylaminocarbonylamino-alkyl, aralkylaminocarbonylamino-alkyl, alkylsuphonylamino-alkyl or arylsulphonylamino-alkyl group;
R₂ represents a carboxyl, alkoxycarbonyl or aralkoxycarbonyl group or a group of the formula: 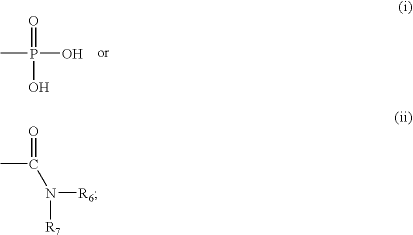
R₃ represents a carboxyl, alkoxycarbonyl or aralkoxycarbonyl group;
R₄ and R₅ each represent a hydrogen atom or R₄ and R₅ together represent an oxo group;
R₆ and R₇ each represent a hydrogen atom or an alkyl or aralkyl group or R₆ and R₇ together with the nitrogen atom to which they are attached represent a saturated 5 membered or 6-membered heteromonocyclic ring which may contain a further nitrogen atom or an oxygen or sulphur atom, and n stands for zero, 1 or 2, and pharmaceutically acceptable salts thereof.

The ACE inhibitors may also include pyroglutamic acid derivatives as disclosed in U.S. Pat. No. 4,234,489 (the entire contents of which are incorporated herein by reference), having the formula (XII) as shown below: 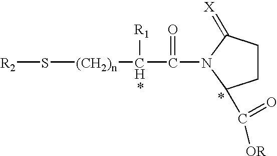
and salts thereof, wherein
R is hydrogen, alkyl or diphenylmethyl;
R₁ is hydrogen, alkyl or trifluoromethyl;
R₂ is hydrogen, 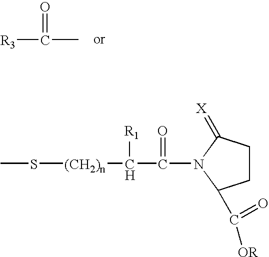
R₃ is hydrogen, alkyl, phenyl, or phenylalkyl;
X is oxygen or sulfur; and
n is 0 or 1.

The ACE inhibitor compounds may also include the phosphonamidate substituted amino or imino acids and salts thereof of U.S. Pat. No. 4,432,971 (the entire contents of which are incorporated herein by reference), and of the formula (XIII) as shown below: 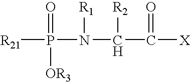
wherein X is an imino or amino acid of the formula: 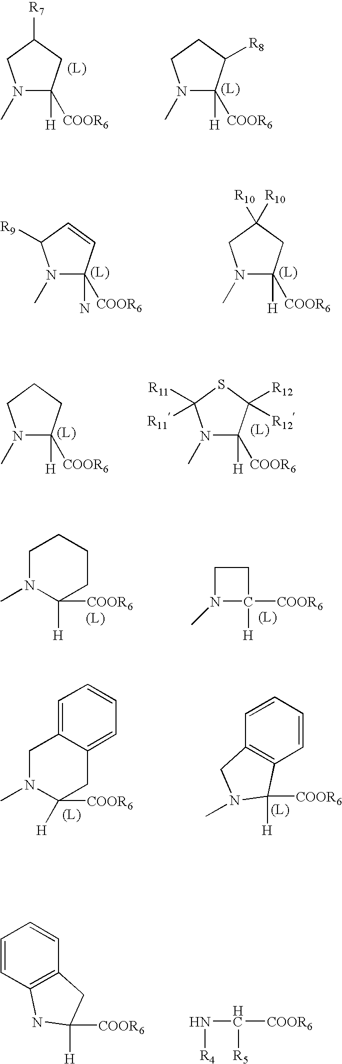
R₇ is hydrogen, lower alkyl, halogen, keto, hydroxyl, 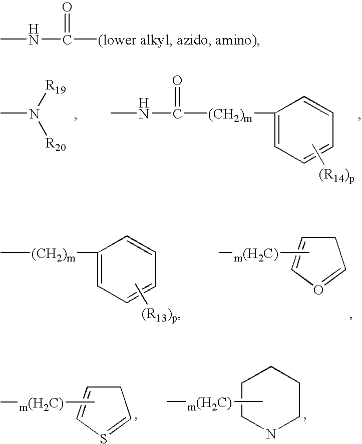
a 1- or 2-naphthyl of the formula: 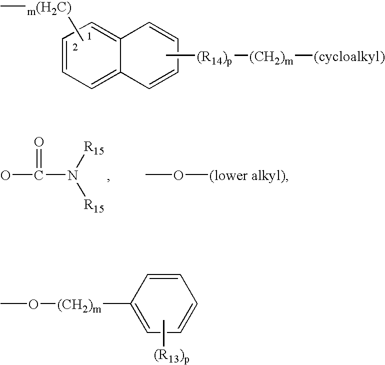
a 1- or 2-naphthyloxy of the formula: 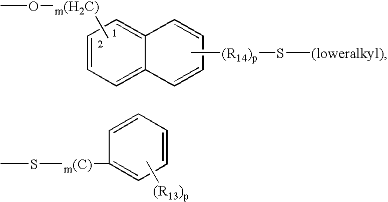
or a 1- or 2-naphthylthio of the formula: 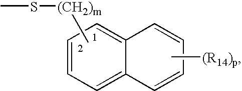
R₈ is keto, halogen, 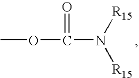 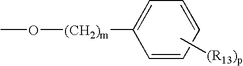
   —O-lower alkyl, a 1- or 2-naphthyloxy of the formula: 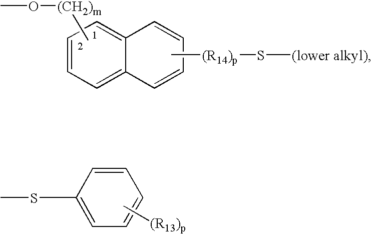
   or a 1- or 2-naphthylthio of the formula: 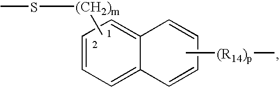
R₉ is keto or 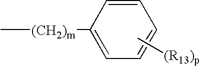
   R₁₀ is halogen or —Y—R₁₆,
   R₁₁, R₁₁, R₁₂ and R₁₂ are independently selected from hydrogen and lower alkyl or R₁₁, R₁₂ and R₁₂ are hydrogen and R₁₁ is 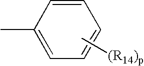
   R₁₃ is hydrogen, lower alkyl of 1 to 4 carbons, lower alkythio of 1 to 4 carbons, chloro, bromo, fluoro, trifluoromethyl, hydroxyl, phenyl, phenoxy, phenylthio, or phenylmethyl.
   R₁₄ is hydrogen, lower alkyl of 1 to 4 carbons, lower alkoxy of 1 to 4 carbons, lower alkylthio of 1 to 4 carbons, chloro, bromo, fluoro, trifluoromethyl, or hydroxy.
   m is zero, one two or three;
   p is one, two or three provided that p is more than one only if R₁₃ or R₁₄ is hydrogen, methyl, methoxy, chloro, or fluoro;
   R₁₅ is hydrogen or lower alkyl of 1 to 4 carbons;
   Y is oxygen or sulfur;
   R₁₆ is lower alkyl of 1 to 4 carbons; 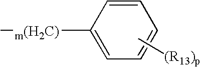
   or the R₁₆ groups join to complete an unsubstituted 5- or 6-membered ring or said ring in which one or more of the carbons has a lower alkyl of 1 to 4 carbons or a di(lower alkyl of 1 to 4 carbons) substituent.
   R₄ is hydrogen, lower alkyl, —(CH₂)ₘ-cycloalkyl, or 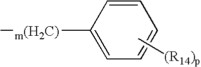
   R₅ is hydrogen, lower alkyl, 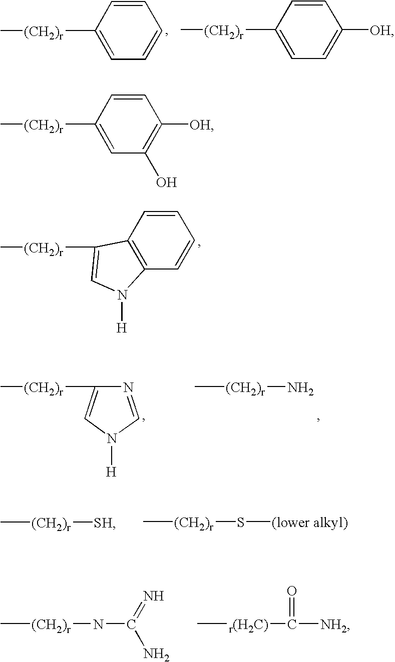
   r is an integer from 1 to 4.
   R₁ is hydrogen, lower alkyl, or cycloalkyl.
   R₂ is hydrogen, lower alkyl, halo substituted lower alkyl, 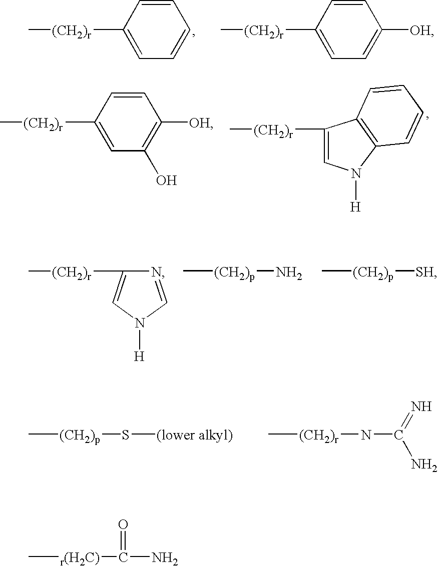
   or R₁ and R₂ taken together are —(CH₂)ₙ— wherein n is an integer from 2 to 4.
R₃ and R₆ are independently selected from hydrogen, lower alkyl, benzyl, benzhydryl, or 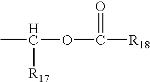
   wherein R₁₇ is hydrogen, lower alkyl, cycloalkyl, or phenyl, and R₁₈ is hydrogen, lower alkyl, lower alkoxy, phenyl, or R₁₇ and R₁₈ taken together are 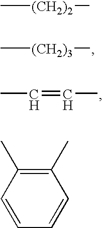
   R₁₉ is lower alkyl, benzyl, or phenethyl;
   R₂₀ is hydrogen, lower alkyl, benzyl or phenethyl;
   R₂₁ is alkyl of 1 to 10 carbons; 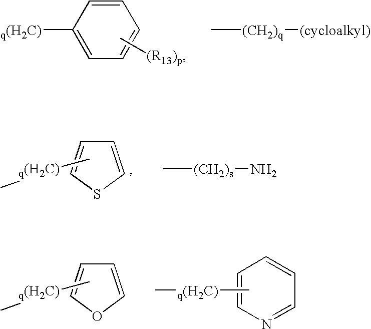
   wherein q is zero or an integer from 1 to 7, s is an integer from 1 to 8, and R₁₃, and p is as defined above.

The ACE inhibitor compounds of the present invention may also include the phosphonate substituted amino or imino acids and salts thereof, as disclosed in U.S. Pat. No. 4,452,790 (the entire contents of which are incorporated herein by reference), of the general formula (XIV) as shown below: 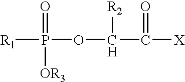
X is an imino or amino acid of the formula: 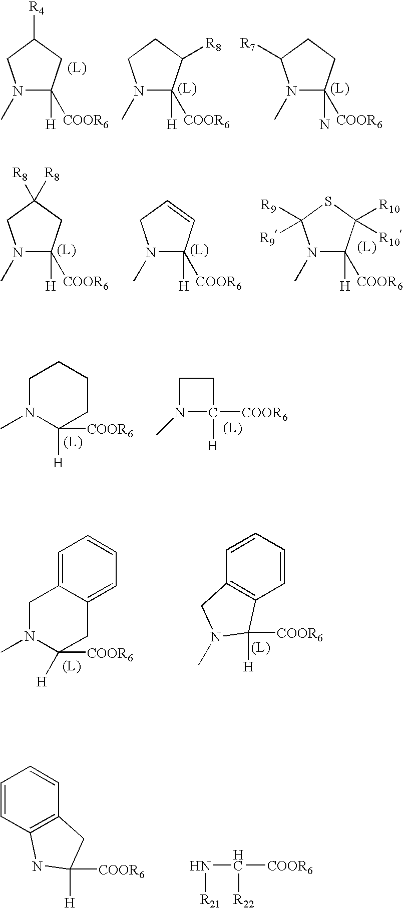
R₄ is hydrogen, lower alkyl, halogen, keto, hydroxy, 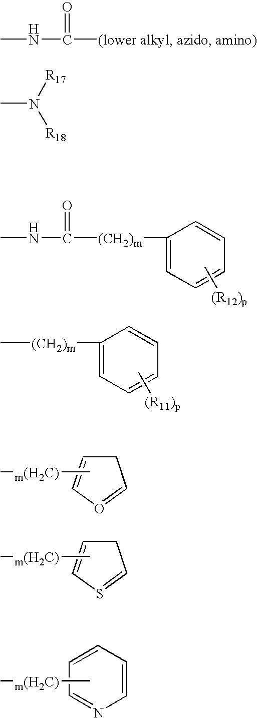
a 1- or 2-naphthyl of the formula: 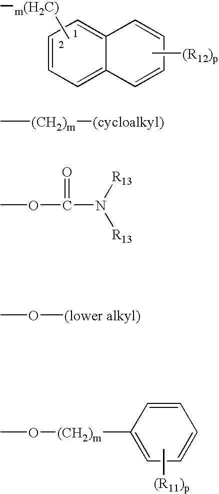
a 1- or 2-naphthyloxy of the formula: 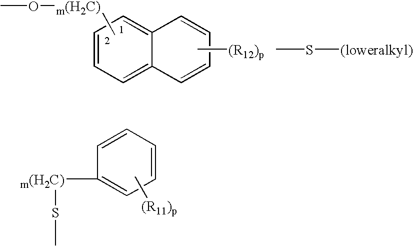
or a 1- or 2-naphthylthio of the formula: 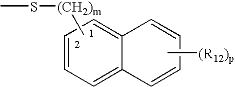
R₅ is keto, halogen, 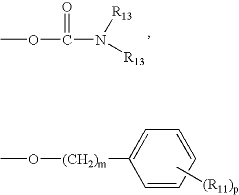
—O-lower alkyl, a 1- or 2-naphthyloxy of the formula: 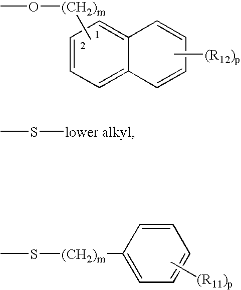
or a 1- or 2-naphthylthio of the formula: 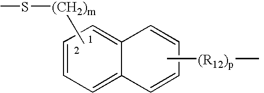
R₇ is keto or 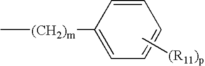
each R₈ is independently halogen or —Y—R₁₄;
R₉, R₉, R₁₀ are independently selected from hydrogen and lower alkyl or R₉, R₁₀ and R₁₀ are hydrogen and R₉ is 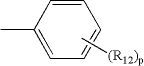
R₁₁ is hydrogen, lower alkyl of 1 to 4 carbons, lower alkoxy of 1 to 4 carbons, lower alkythio of 1 to 4 carbons, chloro, bromo, fluoro, trifluormethyl, hydroxyl, phenyl, phenoxy, phenylthio, or phenylmethyl;
R₁₂ is hydrogen, lower alkyl of 1 to 4 carbons, lower alkoxy of 1 to 4 carbons, lower alkythio of 1 to 4 carbons, chloro, bromo, fluoro, trifluoromethyl, or hydroxy;
m is zero, one two or three;
p is one, two or three provided that p is more than one only if R₁₁ or R₁₂ is hydrogen, methyl, methoxy, chloro, fluoro;
R₁₃ is hydrogen or lower alkyl of 1 to 4 carbons;
Y is oxygen or sulfur;
R₁₄ is lower alkyl of 1 to 4 carbons; 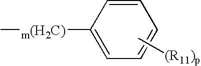
or the R₁₄ groups join to complete an unsubstituted 5- or 6-membered ring or said ring in which one or more of the carbons has a lower alkyl of 1 to 4 carbons or a di(lower alkyl of 1 to 4 carbons) substitutent;
R₂₁ is hydrogen, lower alkyl, cycloalkyl, phenyl, or 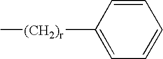
R₂₂ is hydrogen, lower alkyl, 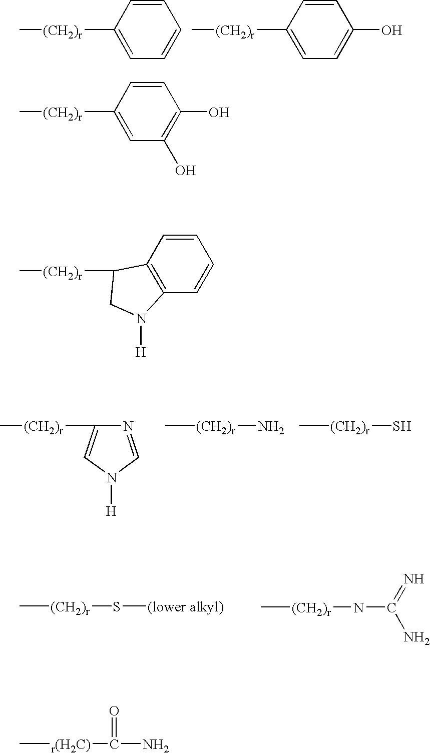
r is an integer from 1 to 4;
R₁ is alkyl of 1 to 10 carbons, aminoalkyl, haloalkyl; 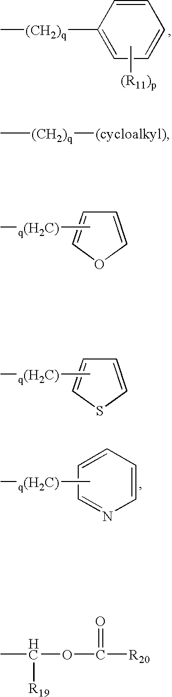
wherein q is zero or an integer from 1 to 7 and R₁₂ and p are defined as above;
R₁₉ and R₂₀ are independently selected from hydrogen, lower alkyl, halo substituted lower alkyl; 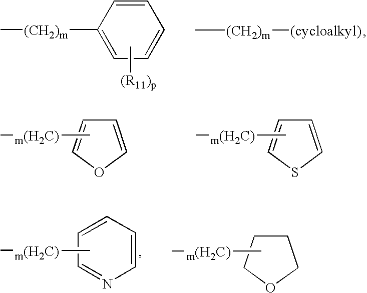
wherein m, R₁₁ and p are as defined above;
R₂ is hydrogen, lower alkyl, halo substituted lower alkyl; 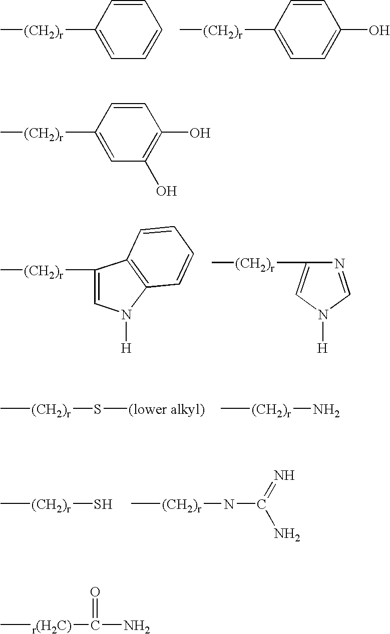
wherein r is defined above.
R₃ and R₆ are independently selected from hydrogen, lower alkyl, benzyl, alkali metal such as Li, Na or K, benzhydryl, or 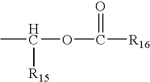
wherein R₁₅ is hydrogen, lower alkyl, cycloalkyl, or phenyl, and R₁₆ is hydrogen, lower alkyl, lower alkoxy, phenyl, or R₁₅ and R₁₆ taken together are —(CH₂)₂—, —(CH₂)₃—, —CH═CH—, or 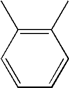
R₁₇ is lower alkyl, benzyl, or phenethyl;
R₁₈ is hydrogen, lower alkyl, benzyl or phenethyl.

The ACE inhibitor compounds may also include the compounds disclosed in EP Patent No. 0060668 (the entire contents of which are incorporated herein by reference), having formula (XV) as shown below: 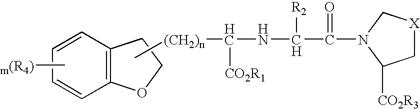
or a pharmaceutically acceptable salt thereof, wherein
m is 0 to 3;
n is 1 to 5;
R₁ is hydrogen or C₁₋₆alkyl;
R₂ is hydrogen, C₁₋₄alkyl, —(CH₂)p-NH₂
wherein p is 1 to 4, or —NHCOR₅ wherein R₅ is C₁₋₄alkyl;
R₃ is hydrogen or C₁₋₆alkyl;
R₄ is C₁₋₄alkyl, C₁₋₄alkoxy, halogen or CF₃; and
X is CH₂ or S;
The dihydrobenzofuranyl moiety may be bonded to the rest of the structure at the 2- or 3-position, preferably, the 2-position;
Preferably X is CH₂.

The ACE inhibitor compounds may also be compounds as disclosed in EP Patent No. 0080822 (the entire contents of which are incorporated herein by reference), having formula (XVI) as shown below: 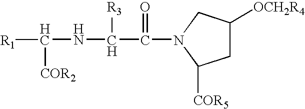
or a pharmaceutically acceptable salt thereof,
wherein R₁ is C₁₋₅ alkyl optionally substituted by NHR_{6,} (wherein R₆ is hydrogen or C₁₋₅) alkylcarbonyl) or by phenyl or naphtyl optionally substituted by halogen, C₁₋₅ alkyl or C₁₋₅ alkoxy or by dihydrobenzofuran-2-yl, optionally substituted in the benzo moiety by C₁₋₅ alkyl, C₁₋₅ alkoxy, halogen or trifluoromethyl;
R₂ and R₅ are the same or different and each is hydroxyl, C₁₋₅ alkoxy, C₂₋₆ alkylcarbonyl or amino optionally substituted by C₁₋₅ alkyl;
R₃ is C₁₋₅ alkyl optionally substituted by the group —NHR₇, wherein R₇ is hydrogen, C₁₋₅ alkyl or C₂₋₆ alkylcarbonyl; and
R₄ is phenyl optionally substituted by halogen, C₁₋₅ alkoxy, trifluromethyl or C₁₋₅ alkyl.

The ACE inhibitor compounds of the present invention may also include phosphory aminoacid derivators as disclosed in EP Patent No. 0009183 (the entire contents of which are incorporated herein by reference), and having the general formula (XVII) as shown below: 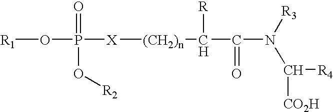
wherein n is 0 or 1;
R is hydrogen, lower alkyl, phenyl lower alkyl, hydroxy phenyl lower alkyl, hydroxy lower alkyl, amino lower alkyl, guanidino lower alkyl, imidazoyl lower alkyl, indolyl lower alkyl, mercapto lower alkyl, lower alkyl mercapto lower alkyl;
R₃ is hydrogen;
R₄ is hydrogen, lower alkyl, phenyl lower alkyl, hydroxylphenyl lower alkyl, hydroxyl lower alkyl, aminolower alkyl, guanidino lower alkyl, guanidino lower alkyl, imidazoyl lower alkyl, indolyl lower alkyl, mercapto lower alkyl, lower alkyl mercapto lower alkyl;
R₃ and R₄ may be connected together to form an alkylene bridge of from 2 to 4 carbon atoms or an alkylene bridge of from 2 to 3 carbon atoms one sulfur atom;
X is O, NR₅, S where R₅═H or lower alkyl;
R₁ is hydrogen, lower alkyl, aralkyl or aryl;
R₂ is hydrogen, lower alkyl, aralkyl or aryl and pharmaceutically acceptable salts thereof.

The ACE inhibitors may also include the carbamate derivatives of mercaptoacyl hydroxylprolines as disclosed in U.S. Pat. No. 4,217,359 (the entire contents of which are incorporated herein by reference), and which have the formula (XVIII) as shown below: 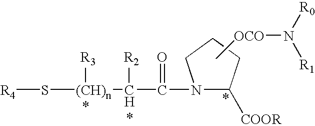
wherein R, R₂ an R₃ each is hydrogen or lower alkyl;
R₀ and R₁ each is hydrogen, lower alkyl, cyclo-lower alkyl, allyl, propargyl, phenyl or substituted phenyl; or R₀ and R₁ can join with the nitrogen to form a 5- or 6-membered heterocyclic;
R₄ is hydrogen or hydrolysable organic protecting group of the formula R₅—CO— or 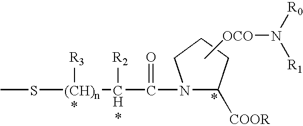
R₅ is lower alkyl, phenyl, substituted phenyl, phenyl-lower alkyl, substituted phenyl-lower alkyl, cycloalkyl, thienyl, or furyl;
n is 0, 1 or 2; and salts thereof.

The asterisks indicate centers of asymmetry. The carbon in the acyclic side chain is asymmetric when R₂ and/or R₃ is other than hydrogen. Each of the centers of asymmetry provide D and L forms which can be separated by conventional methods as described below.

The ACE inhibitors of the present invention also may include substituted acyl derivatives of amino acids disclosed in U.S. Pat. No. 4,129,571 (the entire contents of which are incorporated herein by reference), and which have the general formula (XIX) as shown below: 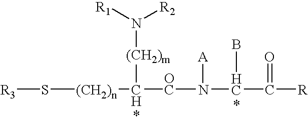
and salts thereof, wherein:
R is hydroxyl or lower alkoxy;
R₁ is hydrogen, lower alkanoyl or amino(imino)-methyl;
R₂ is hydrogen, lower alkyl or phenyl-lower alkylene;
R₃ is hydrogen, lower alkanoyl, benzoyl or 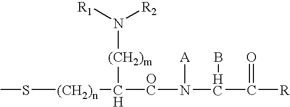
A is hydrogen, lower alkyl or hydroxyl-lower alkylene;
B is hydrogen, lower alkyl, phenyl, phenyl-lower alkylene, hydroxyl-lower alkylene, hydroxyphenyl-lower alkylene, amino-lower alkylene, guanidino-lower alkylene, mercapto-lower alkylene, lower alkyl-thio-lower alkylene, imidazolyl-lower alkylene, indolyl-lower alkylene, carbamoyl-lower alkylene or carboxy-lower alkylene;
or A and B together form a (CH₂)ₚ bridge which completes a ring of 5 or 6 atoms with the nitrogen and carbon to which they are joined, one carbon optionally bearing a hydroxy group;
n is 0 or 1;
m 0, 1, 2, 3 or 4; at least one of m and n is other than 0; and
p is 3 or 4.

The asterisks denote centers of asymmetry.

ACE inhibitors may also include halogenated compounds as disclosed in U.S. Pat. No. 4,154,935 (the entire contents of which are incorporated herein by reference), which have the general formula (XX) as shown below: 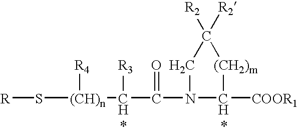
wherein R is hydrogen, lower alkanoyl or 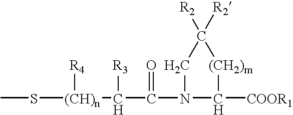
R₁ is hydrogen or lower alkyl;
R₂ and R₂ each independently represent hydrogen or halogen;
R₃ and R₄ each independently represent hydrogen, lower alkyl or trifluoromethyl, not more than one being trifluoromethyl, and at least one of R₂, R_{2′}, R₃ or R₄ is a halogen or trifluoromethyl substituent represented by the named symbol as defined above;
m is 2; and
n is 0 or 1.

The asterisks indicate asymmetric carbon atoms.

ACE inhibitor compounds may also include carboxyalkylacylamino acids and related compounds disclosed in U.S. independently represent 4,052,511 (the entire contents of which are incorporated herein by reference), which are derivatives of proline, pipecolic acid, azetidine-2-carboxylic acid and which have the general formula (XXI) as shown below: 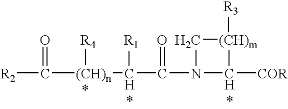
wherein
R is hydroxy, amino or lower alkoxy;
R₁ and R₄ each is hydrogen, lower alkyl or phenyl-lower alkyl;
R₂ is hydroxy, amino, hydroxyamino or lower alkoxy;
R₃ is hydrogen, hydroxy or lower alkyl;
m is 1 to 3;
n is 0 to 2.

The asterisks indicate asymmetric carbon atoms. The carbons in the acyclic side chain are asymmetric when R₁ or R₄ are other than hydrogen.

The ACE inhibitor compounds may also include the dehydrocyclicimino acid compounds disclosed in U.S. independently represent U.S. Pat. No. 4,129,566 (the entire contents of which are incorporated herein by reference), which have the general formula (XXII) as shown below: 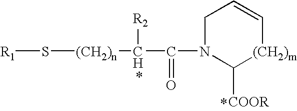
wherein:
R and R₂ each is hydrogen or lower alkyl;
R₁ is hydrogen, lower alkanoyl or 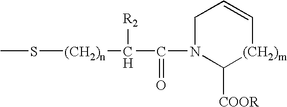
m and n each is 0 or 1.

The asterisks indicate asymmetric carbon atoms. The carbon in the acyclic side chain is asymmetric when R₁ is other than hydrogen.

The ACE inhibitor compounds may also include compounds disclosed in U.S. independently represent 4,053,651 (the entire contents of which are incorporated herein by reference), having the formula (XXIII) as shown below: 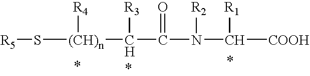
or a salt thereof, wherein
R₁ is hydrogen, lower alkyl, phenyl-lower alkylene, hydroxyl-lower alkylene, amino-lower alkylene, guanidino-lower alkylene, imidazolyl-lower alkylene, indolyl-lower alkylene, mercapto-lower alkylene, lower alkylmercapto-lower alkylene, carbamoyl-lower alkylene or carboxy-lower alkylene;
R₂, R₃ and R₄ each is hydrogen, lower alkyl or phenyl-lower alkylene;
R₅ is hydrogen, lower alkanoyl, benzoyl or 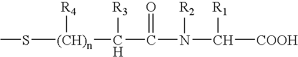
n is 0, 1 or 2.

The asterisks denote centers of asymmetry.

ACE inhibitors may also include derivatives of mercaptoacyl prolines and pipecolic acids as disclosed in U.S. independently represent 4,311,697 (the entire contents of which are incorporated herein by reference), of the formula (XXIV) as shown below: 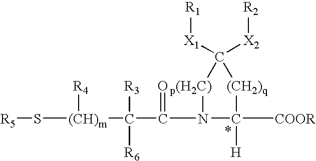
R and R₆ are independently selected from hydrogen and lower alkyl provided that R₆ is lower alkyl only if R₃ is also lower alkyl;
R₃ and R₄ are independently selected from hydrogen, lower alkyl, lower alkylthio, —(CH₂)ₙ—SH, and halo substituted lower alkyl;
X₁, X₂ and X₃ are independently selected from lower alkyl, lower alkenyl, lower alkynl, cycloalkyl, halo substituted lower alkyl, hydroxyl substituted lower alkyl; 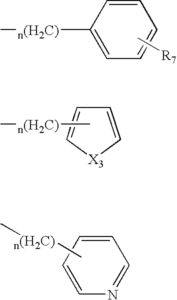
or R₁ and R₂ join in a polymethylene chain to complete an unsubstituted or substituted 5- or 6-membered ring.

When R₁ and R₂ are joined together in a polymethylene chain of 2 or 3 carbons, these cyclic ketal and thioketals can be represents as follows: 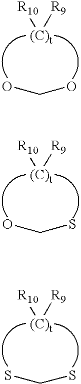
wherein t is 2 or 3 and R₉ and R₁₀ are both hydrogen, both lower alkyl, or one is hydrogen and the other is lower alkyl, halo substituted lower alkyl, hydroxyl substituted lower alkyl, 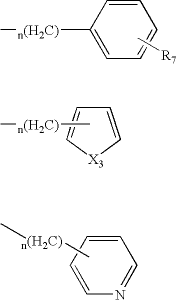
preferably, only one carbon of the polymethylene chain will be substituted.
R₇ is hydrogen, lower alkyl of 1 to 4 carbons, especially methyl, lower alkoxy of 1 to 4 carbons, especially methyl, lower alkoxy of 1 to 4 carbons, especially methoxy, lower alkythio 1 to 4 carbons, especially methylthio, chloro, bromo, fluoro, trifluoromethyl, or hydroxy;
R₅ is hydrogen, a hydrolyzably removable protecting group, a chemically removable protecting group, or when R₃ and R₄ are other than —(CH₂)ₘ—SH a sulfide of the formula 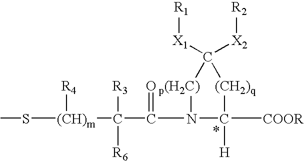
m is zero, one or two;
n is one, two or three;
p and q are each one or two provided that both are not two.

The asterisk in the above formula indicates a center of asymmetry in the ring.

In the case of praline, i.e., p and q are both one, this center is in the L-configuration.

In the case of pipecolic acid, i.e., one of p and q is two, this center is in the D, L or L-configuration.

Asymmetric centers can also be present in the mercaptoacyl sidechain depending upon the definition of R₃, R₄ and R₆. Another asymmetric center may also be present in the ring when X₁—R₁ and X₂—R₂ are different. The products can accordingly exist in stereoisomeric forms or as racemic mixtures thereof.

The ACE inhibitors may also include the imido, amido and amino derivative compounds of mercaptoacyl prolines and pipecolic acids disclosed in U.S. Pat. No. 4,310,461 (the entire contents of which are incorporated herein by reference), and having the formula (XXV) as shown below: 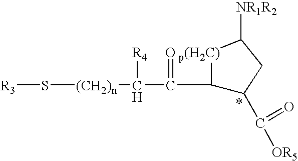
and salts thereof, and the symmetrical dimer thereof, wherein
R₁ and R₂ are the same or different and are hydrogen, alkyl, cycloalkyl, 1-adamantyl, aryl, arylalkyl, alkylcarbonyl, arylcarbonyl, arylalkylcarbonyl, alkysulfonyl, arysulfonyl, or arylvinylcarbonyl (aryl-CH═CH—O—), or together with the nitrogen atom to which they are attached R₁ and R₂ are 1-pyrrolidinyl, 1-piperidinyl, 1-homopiperidinyl, 4-morpholinyl, 4-alkyl-1-piperazinyl, 4-aryl-1-piperazinyl, 1-imidazolyl, 1-pyrrolidinyl-2,5-dione(succinimido), 3-alkyl-1-pyrrolidinyl-2,5-dione, 3-aryl-1-pyrrolidinyl-2,5-dione, 1-piperidinyl-2,6-dione, 2H-isoindol-2-yl-1,3-dione(pthalimido), hexahydro-2H-isoindol-2-yl-1,3-dione(hexahydrophthalimido), 2,5-dihydro-2,5-dioxo-1H-pyrrol-1-yl(maleimido), 1,1,3-trioxo-1,2-benziso-thiazol-2 (3H)-yl(2-saccharinyl), or 1,3-dihydro-1,3-dioxo-2H-benz[de]isoquinolin-2-yl(1,8-naphthalenedicarboximdo);
R₃ is hydrogen, alkyl, aryl, arylalkyl, or a hydrolyzable acyl protecting group such as alkanoyl or arylcarbonyl;
R₄ is hydrogen, alkyl, alkythio or trifluoromethyl;
R₅ is hydrogen, alkyl, or arylalkyl;
n is 0, 1 or 2; and p is 1 or 2.

The ACE inhibitors may also include phosphonoacyl prolines and related compounds as disclosed in U.S. Pat. No. 4,151,172 (the entire contents of which are incorporated herein by reference), which have the formula (XXVI) as shown below: 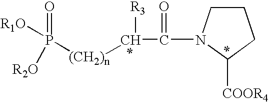
R₁ and R₂ each is hydrogen, lower alkyl, lower alkenyl, unsubstituted or substituted phenyl-lower alkyl or a metal ion;
R₃ is hydrogen or lower alkyl;
R₄ is hydrogen, lower alkyl, phenyl-lower alkyl or a metal ion; and
n is 0 or 1.

The ACE inhibitor compounds may also include mercaptoacyl derivatives of various 4-cis substituted prolines and salts thereof as disclosed in U.S. Pat. No. 4,316,905 (the entire contents of which are incorporated herein by reference), and which have the formula (XXVII) as shown below: 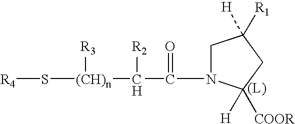
R represents hydrogen or lower alkyl;
R₁ represents-(CH₂)m-cycloalkyl, 1-cyclohexenyl, 1,4-cyclohexadienyl; 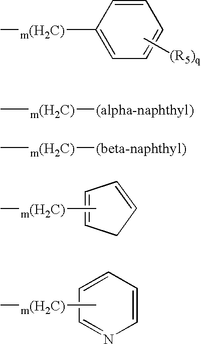
R₂ and R₃ are independently selected from hydrogen, lower alkyl, lower alkylthio and halo substituted lower alkyl;
n is zero, one or two;
R₄ is hydrogen, a hydrolyzably removable protecting group, a chemically removable protecting group, or 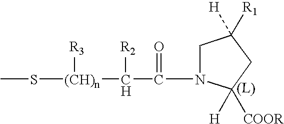
m is zero, one, two or three;
R₅ is hydrogen, lower alkyl of 1 to 4 carbons, especially methyl, lower alkoxy of 1 to 4 carbons, especially methoxy, lower alkylthio of 1 to 4 carbons, especially methylthio, chloro, bromo, fluoro, trifluoromethyl, hydroxy, phenyl, phenoxy, phenylthio, or phenylmethyl. The hydroxy substituted compounds are obtained by heating the corresponding methoxy substituted compound with pyridine HCl;
q is one, two or three provided that q is more than one only if R₅ is hydrogen, methyl, methoxy, chloro or fluoro;
X is oxygen or sulfur.

The ACE inhibitors also preferably include: aylmercapto and mercaptoalkanoyl prolines (U.S. Pat. No. 4,046,889) such as captopril (1-[(2S)-3-mercapto-2-methylpropionyl]-L-proline) (U.S. Pat. No. 4,105,776) and ether or thioether mercaptoacyl prolines such as zofenopril (U.S. Pat. No. 4,316,906); carboxyalkyl dipeptides such as enalapril (N-(1-ethoxycarbonyl-3-phenylpropyl)-L-ananyl-L-proline) (U.S. Pat. No. 4,374,829), lisinopril (U.S. Pat. No. 4,374,829), quinapril (U.S. Pat. No. 4,344,949), and ramipril (U.S. Pat. No. 4,508,729); carboxyalkyl dipeptide mimics such as cilazapril (U.S. Pat. No. 4,512,924) and benazapril (U.S. Pat. No. 4,410,520); phosphinylalkanoyl prolines (U.S. Pat. No. 4,168,267) such as fosinopril (U.S. Pat. No. 4,337,201) and trandolopril; phosphonamidate substituted amino or imino acids (U.S. Pat. No. 4,432,971); phosphonate substituted amino or imino acids and salts thereof, including ceronapril ((S)-1-[6-amino-2-[[hydroxyl(4-phenylbutyl)phosphinyl]oxy]-1-oxohexyl]-L-proline) (U.S. Pat. No. 4,452,790); Beecham's BRL 36,378 (EP Nos. 80822 and 60668); Chugai's MC-838 (disclosed in CA 102:72588v and Jap. J. Pharmacol. 40:373 (1986)); Ciba-Geigy's CGS 14824 (3-([1-ethoxycarbonyl-3-phenyl-(1S)-propyl]-amino)-2,3,4,5-tetrahydro-2-oxo-1-(3S)-benzazepine-1 acetic acid HCL) (U.K. Patent No. 2103614) and CGS 16,617 (3(S)-[[(1S)-5-amino-1-carboxypentyl]amino]2,3,4,5-tetrahydro-2-oxo-1H-1-benzazepine-1-ethanoic acid) (U.S. Pat. No. 4,473,575); Cetapril (alacepril, Dainippon) (Eur. Therap. Res. 39:671 (1986); 40:543 (1986)); Ru 44570 (Hoechst) (Arzneimittelforschung 35:1254 (1985)); Cilazapril (Hoffman-LaRoche) (J. Cardiovasc. Pharmacol. 9:39 (1987); Ro 31-2201 (Hoffman-LaRoche) (FEBS Lett. 165:201 (1984); Lisinopril (Merck) (Curr. Therap. Res. 37:342 (1985) and Eur. Patent App. No. 12-401); Indalapril (delapril) (U.S. Pat. No. 4,385,051); Rentiapril (fentiapril, Santen) (Clin. Exp. Pharmacol. Physiol. 10:131 (1983); Indolapril (Schering) (J. Cardiovasc. Pharmacol. 5:643, 655 (1983)); Spirapril (Schering) (Acta. Pharmacol. Toxicol. 59 (Supp. 5):173 (1986); Perindopril (Servier) (Eur. J. Clin. Pharmacol. 31:519 (1987); Quinapril (Warner-Lambert) (U.S. Pat. No. 4,344,949); CI 925 (Warner-Lambert) ([3S-[2[R(*)R(*)]]3R(*)]-2-[2-[[1-(ethoxy-carbonyl)-3-phenylpropyl]amino[-1-oxopropyl]-1,2,3,4-tetrahydro-6,7-dimethoxy-3-isoquinolinecarboxylic acid HCL) (Pharmacologist 26:243, 266 (1984)); WY-44221 (Wyeth) (J. Med. Chem. 26:394 (1983)); Mercapto containing compounds such as pivopril and YS980 (U.S. Pat. No. 6,127,370); Omapatrilat (Drugs R. D. 1999 1 (4):350-1) (U.S. Pat. No. 6,300,503); Alacepril (Jap. Patent App. No. 78/82809); moveltopril; quinaprilat; moexipril; perinodpril (S-9490) (Annual Drug Data Report 7, 99 (1985)); pentopril; ancovenin (Annual Drug Data Report 6, 20 (1984)); phenacein (Annual Drug Data Report 7, 20 (1985)); and nicotianamin (East German Patent DD 226 880). The contents of all cited patents, applications, publications are hereby incorporated herein by reference.

The most preferred ACEI is Enalapril, i.e. (N-(1-ethoxycarbonyl-3-phenylpropyl)-L-ananyl-L-proline), (U.S. Pat. No. 4,374,829, entire contents incorporated herein by reference) and other similar or derivative carboxyalkyl dipeptides. Other preferred ACE inhibitors include lisinopril or captopril.

(iii) Angiotensin II Receptor Blockers/Antagonists

Angiotensin is formed from a precursor, angiotensinogen, which is produced by the liver and found in the alpha-globulin fraction of plasma. The lowering of blood pressure is a stimulus to secretion of renin by the kidney into the blood. Renin cleaves from angiotensinogen a terminal decapeptide, angiotensin I. This is further altered by the enzymatic removal of a dipeptide, by Angiotensin Convertin Enzyme (ACE), to form angiotensin II. Angiotensin II is a potent regulator of blood pressure and of water and electrolyte balance. Angiotensin II interacts with two pharmacologically distinct subtypes of cell surface receptors, types 1 and 2. Whereas AGTR1, the type-1 receptor for Angiotensin II, mediates the vasopressive and aldosterone-secreting effects of angiotensin II, the function of the type-2 Angiotensin receptor (AGTR2) was relatively unclear, although it is expressed in both adult and embryonic life. Recent evidence indicates that the type-2 Angiotension receptor is not required for embryonic development, but plays a role in the central nervous system and cardiovascular functions that are mediated by the renin-angiotensin system (Hein et al., Nature 377: 744-748, 1995).

Ichiki et al. (Nature 377: 748-750, 1995) reported the unexpected finding that the targeted disruption of the mouse AGTR2 gene resulted in a significant increase in blood pressure and increased sensitivity to the pressor action of angiotensin II. The authors concluded that the type 2 receptor mediates a depressor effect and antagonizes the AGTR1-mediated pressor action of angiotensin II. In addition, disruption of the AGTR2 gene attenuated exploratory behavior and lowered blood pressure. Their results indicated that angiotensin II activates AGTR1 and AGTR2, which have mutually counteracting hemodynamic effects, and that AGTR2 regulates central nervous system functions, including behavior. Ichiki et al. (1995) commented on the fact that Hein et al. (1995) did not find an increase in basal blood pressure and they suggested that this could be due to differences in genetic background of the mice studied.

Angiotensin-II receptor antagonists act by binding to specific membrane-bound receptors that displace Angiotensin II from its type 1-receptor subtype (AGTR1). These drugs therefore function as selective blockers. AT-II pressor effects are mediated by AGTR1. Unlike angiotensin-converting enzyme inhibitors, they do not inhibit bradykinin metabolism or enhance prostaglandin synthesis. Angiotensin-II receptor antagonists are well tolerated. Cough occurs much less often with these agents than with angiotensin-converting enzyme inhibitors, and they do not adversely affect lipid profiles or cause rebound hypertension after discontinuation. There has been a rapid growth in members of this new class of drugs. The angiotensin-II receptor antagonists that have been labeled for use in hypertension by the U.S. Food and Drug Administration (FDA) are Losartan (Cozaar®), Valsartan (Diovan®), Irbesartan (Avapro®), Candesartan (Atacand®) and Telmisartan (Micardis®). Other angiotensin-II receptor antagonists currently under investigation include tasosartan, zolarsartan, Teveten (eprosartan mesylate). At the present time four are being actively marketed in Canada: Losartan (Cozaar®), Valsartan (Diovan®), Irbesartan (Avapro®), Candesartan (Atacand®).

Losartan. Losartan (U.S. Pat. No. 5,153,197) was the first angiotensin-II receptor antagonist to be introduced (1995). Compared with the parent drug, the active metabolite (EXP3174) has a longer half-life and antihypertensive effects that correlate more with plasma concentration. Double-blind studies have shown that losartan is well tolerated and as efficacious as enalapril and nifedipine for lowering blood pressure. The mean blood pressure reduction achieved with losartan in a dosage of 50 to 150 mg once daily is 5.5 to 10.5 mm Hg for systolic pressure and 3.5 to 7.5 mm Hg for diastolic pressure. One review of the efficacy and safety of losartan in the treatment of essential hypertension indicated a slowly developing response, with blood pressure becoming lower over several weeks of continued treatment.

The starting dosage of losartan is 50 mg once daily. The duration of activity for a dose is 24 hours. Twice-daily dosing can be used if the antihypertensive effect measured at a trough is inadequate. However, a comparison of losartan in dosages of 100 mg once daily and 50 mg twice daily showed no significant difference in antihypertensive efficacy.

A hydrochlorothiazide-losartan combination (Hyzaar) is also available. This combination drug contains 12.5 mg of hydrochlorothiazide and 50 mg of losartan. Some investigators advocate the use of this combination instead of escalation of a single drug, because dose-dependent adverse effects are less likely to occur. Dosing is once or twice daily.

Valsartan. (U.S. Pat. Nos. 5,399,578; 6,294,197) Placebo-controlled trials have found valsartan to be both safe and effective for the treatment of hypertension. With valsartan taken in a dosage of 80 to 320 mg once daily, the mean reduction in diastolic blood pressure is 6 to 9 mm Hg, and the mean reduction in systolic pressure is 3 to 6 mm Hg. Studies have shown that valsartan is as effective as ACE inhibitors enalapril, lisinopril and amlodipine in the treatment of mild to moderate hypertension.

The affinity of valsartan for the AT₁ receptor is about 20,000 times greater than its affinity for the AT₂ receptor. In comparison, the affinity of losartan for the AT₁ receptor is about 1,000 times greater than its affinity for AT₂ receptors. The clinical implication of receptor affinity is not yet clear.

Valsartan is also available as a combination product with hydrochlorothiazide (Diovan HCT). This combination drug contains 80 or 160 mg of valsartan and 12.5 mg of hydrochlorothiazide. With the addition of hydrochlorothiazide, blood pressure decreases even more (i.e., by 6 mm Hg systolic and 3 mm Hg diastolic). Dosing is once daily.

Irbesartan. Irbesartan (U.S. Pat. Nos. 5,270,317; 5,994,348; 6,342,247) is a safe and effective angiotensin-II receptor antagonist with an affinity for the AT₁ receptor that is more than 8,500 times greater than its affinity for the AT₂ receptor. This agent has a higher bioavailability (60 to 80 percent) than other drugs in its class.

In one study, 530 patients with mild to moderate hypertension were given placebo, losartan in a dosage of 100 mg per day or irbesartan in a dosage of 150 or 300 mg per day. After only one week of therapy, blood pressure trough reduction was significantly greater with irbesartan in a dosage of 300 mg per day than with losartan in a dosage of 100 mg per day.

Placebo-controlled trials have shown that irbesartan in a dosage of 150 to 300 mg per day lowers mean systolic blood pressure by 8 to 12 mm Hg and mean diastolic pressure by 5 to 8 mm Hg. Irbesartan has also been found to be as effective as enalapril and atenolol in reducing blood pressure.

A combination product that contains both irbesartan and hydrochlorothiazide is being developed.

Candesartan. Candesartan cilexetil (U.S. Pat. No. 5,196,444) has been shown to be effective for the treatment of hypertension. Candesartan itself is poorly absorbed after oral administration; the ester prodrug, candesartan cilexetil, improves bioavailability. With oral administration of candesartan cilexetil, conversion to the active compound occurs rapidly and completely during gastrointestinal absorption. The affinity of candesartan for the AT₁ receptor is more than 10,000 times greater than its affinity for the AT₂ receptor.

Candesartan is both safe and well tolerated in dosages of 8 to 32 mg per day. With these dosages, systolic blood pressure is reduced by 8 to 12 mm Hg and diastolic pressure is reduced by 4 to 8 mm Hg.

Comparable reductions of diastolic blood pressure have been achieved with candesartan in a dosage of 8 mg per day and enalapril in a dosage of 10 mg per day. In one trial, significant reductions in mean sitting diastolic pressures occurred after 12 weeks of treatment with candesartan in a dosage of 8 or 12 mg per day and enalapril in a dosage of 10 mg per day (P<0.01), but not with candesartan in a dosage of 4 mg per day (P=0.074). The same study compared losartan in a dosage of 50 mg per day with candesartan in dosages of 8 and 16 mg per day. The 16-mg dosage of candesartan reduced diastolic blood pressure by an adjusted mean of 3.7 mm Hg more than the 50-mg losartan dosage.

Telmisartan. Telmisartan (U.S. Pat. No. 5,591,762) is the most recently labeled angiotensin-II receptor antagonist. Its affinity for the AT₁ receptor is more than 3,000 times greater than its affinity for the AT₂ receptor. Nonlinear pharmacokinetics yield a greater than proportional increase in plasma telmisartan concentrations with increasing dosages.

The efficacy of telmisartan in the treatment of hypertension has been demonstrated in placebo-controlled trials. A three-month study of 440 patients showed that telmisartan in a dosage of 40, 80, 120 or 160 mg per day produced a slightly greater antihypertensive effect than enalapril in a dosage of 20 mg per day. In this study, diastolic blood pressure reductions with telmisartan ranged from 8.6 to 9.3 mm Hg, and systolic blood pressure reductions ranged from 10 to 11.9 mm Hg. The decreases in diastolic and systolic blood pressures for enalapril were 7.2 and 8.2 mm Hg, respectively.

Like the other angiotensin-II receptor antagonists, telmisartan has been shown to have a side effect profile similar to that of placebo. Clinical trials have demonstrated no rebound hypertension or first-dose orthostatic effect.

Most recently, the FDA has approved a new angiotensin II receptor blocker called olmesartan medoxomil (Benicar), for the treatment of hypertension. A 20 mg-starting dose of olmesartan medoxomil has been shown to reduce systolic pressure by an average of 15 mm Hg and diastolic pressure by an average of 12 mm Hg. The manufacturers Sankyo Pharma Inc. stated that studies have shown their drug to be superior to losartan, and the launch of Benicar is expected within the first half of 2002.

In addition, Iyer et al. (Proc. Nat. Acad. Sci. 93: 9960-9965, 1996) explored the possibility of using gene therapy to inhibit AGTR1. They demonstrated that the delivery of angiotensin type 1 receptor antisense by a retrovirally-mediated delivery system resulted in a selective attenuation of the cellular actions of angiotensin II in the neurons of the spontaneously hypertensive (SH) rat model. A single injection normalized blood pressure in the SH rat on a long-term basis. The use of this approach in patients was proposed. Thus, this type of AGTR1 antagonist is also within the scope of the AIIRA of the instant invention.

Although both ACE inhibitors and AIIRAs prevent the activation of AGTR1, there is a difference between the effects of these two classes of compounds. This is because non-ACE pathways can also produce some angiotensin II. ACE inhibitors also decrease bradykinin breakdown and this action could be involved in some of the beneficial and adverse effects of that class of drugs. Therefore, a potential for differential clinical effects exists for these two classes of drugs. For example, angiotensin receptor blockers are indicated in patients who require an ACE inhibitor but who cannot tolerate it due to drug-induced dry cough. Similar consideration can also be helpful in conjoint administration of morphogen with either ACE inhibitor or AIIRA.

B. Formulations and Methods of Treatment

In one embodiment, the invention comprises a pharmaceutical composition comprising a therapeutically effective amount an ACE inhibitor and an OP/BMP morphogen formulated with pharmaceutically acceptable salt, carrier, excipient or diluent. In one embodiment, the ACE inhibitor is Enalapril. In another embodiment, the ACE ACEI is: any one compound of the formulas I-XXVIII or their salts thereof; acylmercapto and mercaptoalkanoyl prolines; captopril (1-[(2S)-3-mercapto-2-methylpropionyl]-L-proline); ether or thioether mercaptoacyl prolines; zofenopril; carboxyalkyl dipeptides; enalapril (N-(1-ethoxycarbonyl-3-phenylpropyl)-L-ananyl-L-proline); lisinopril; quinapril; ramipril; carboxyalkyl dipeptide mimics; cilazapril; benazapril; phosphinylalkanoyl prolines; fosinopril; trandolopril; phosphonamidate substituted amino or imino acids; phosphonate substituted amino or imino acids and salts thereof; ceronapril ((S)-1-[6-amino-2-[[hydroxy](4-phenylbutyl)phosphinyl]oxy]-1-oxohexyl]-L-proline); BRL 36,378; MC-838; CGS 14824 (3-([1-ethoxycarbonyl-3-phenyl-(1S)-propyl]-amino)-2,3,4,5-tetrahydro-2-oxo-1-(3S)-benzazepine-1 acetic acid HCL); CGS 16,617 (3(S)-[[(1S)-5-amino-1-carboxypentyl]amino]2,3,4,5-tetrahydro-2-oxo-1H-1-benzazepine-1-ethanoic acid); Cetapril (alacepril, Dainippon); Ru 44570; Cilazapril; Ro 31-2201; Lisinopril; Indalapril (delapril); Rentiapril (fentiapril, Santen); Indolapril; Spirapril; Perindopril; Quinapril; CI 925 ([3S-[2[R(*)R(*)]]3R(*)]-2-[2-[[1-(ethoxy-carbonyl)-3-phenylpropyl]amino[-1-oxopropyl]-1,2,3,4-tetrahydro-6,7-dimethoxy-3-isoquinolinecarboxylic acid HCL); WY-44221; mercapto-containing compounds; pivopril; YS980; Omapatrilat; Alacepril; moveltopril; quinaprilat; moexipril; perinodpril (S-9490); pentopril; ancovenin; phenacein; or nicotianamin.

In another embodiment, the invention comprises a pharmaceutical composition comprising a therapeutically effective amount an AIIRA and an OP/BMP morphogen formulated with pharmaceutically acceptable salt, carrier, excipient or diluent. In one embodiment AIIRA is: Losartan (Cozaar®), Valsartan (Diovan®), Irbesartan (Avapro®), Candesartan (Atacand®), Telmisartan (Micardis®), tasosartan, zolarsartan, Teveten (eprosartan mesylate) or olmesartan medoxomil (Benicar).

In one embodiment, the morphogen in any of the above pharmaceutical composition embodiments is the polypeptide of SEQ ID NO: 3.

In another embodiment, the morphogen in any of the above pharmaceutical composition embodiments is a first polypeptide including at least a C-terminal cysteine domain of a protein selected from: a pro form, a mature form, or a soluble form of a second polypeptide, wherein said second polypeptide is: OP-1, OP-2, OP-3, BMP2, BMP3, BMP4, BMP5, BMP6, or BMP9.

In another embodiment, the morphogen in any of the above pharmaceutical composition embodiments comprises a polypeptide having at least 70% homology or 50% identity with an amino acid sequence of a C-terminal seven-cysteine domain of human OP-1 (SEQ ID NO: 2). In another embodiment, the polypeptide has at least 75% homology or 60% identity with an amino acid sequence of a C-terminal seven-cysteine domain of human OP-1 (SEQ ID NO: 2). In yet another embodiment, the polypeptide has at least 80% homology or 70% identity with an amino acid sequence of a C-terminal seven-cysteine domain of human OP-1 (SEQ ID NO: 2). In yet another embodiment, the polypeptide has at least 90% identity with an amino acid sequence of a C-terminal seven-cysteine domain of human OP-1 (SEQ ID NO: 2).

The invention also comprises a package pharmaceutical comprising any of the pharmaceutical compositions described herein, in association with instructions for administering the composition to a mammal for treatment or prevention of chronic renal failure.

The invention also comprises a package pharmaceutical comprising any of the pharmaceutical compositions described herein, in association with instructions for administering the composition to a mammal for delaying the need or reducing the frequency of chronic dialysis treatments.

Iin one embodiment, the invention provides a method of treating or preventing chronic renal failure in a mammal, comprising conjointly administering to said mammal (i) an OP/BMP morphogen, an inducer of endogenous OP/BMP morphogen expression, or an agonist of an OP/BMP morphogen receptor; and (ii) an Angiotensin-Converting Enzyme Inhibitor (ACEI).

In one embodiment, the invention provides a method of treating or preventing chronic renal failure in a mammal, comprising conjointly administering to said mammal (i) an OP/BMP morphogen, an inducer of endogenous OP/BMP morphogen expression, or an agonist of an OP/BMP morphogen receptor; and (ii) an Angiotensin-II Receptor Antagonist (AIIRA).

In another embodiment, the invention provides a method of treating or preventing chronic renal failure in a mammal, comprising introducing into the kidney of said mammal a therapeutically effective amount of renal mesenchymal progenitor cells pre-treated conjointly with an ACEI and an agent that increases the abundance of an OP/BMP morphogen.

In another embodiment, the invention provides a method of treating or preventing chronic renal failure in a mammal, comprising introducing into the kidney of said mammal a therapeutically effective amount of renal mesenchymal progenitor cells pre-treated conjointly with an AIIRA and an agent that increases the abundance of an OP/BMP morphogen. In one embodiment the agent is an OP/BMP morphogen. In another embodiment the agent is an inducer of an OP/BMP morphogen. In another embodiment the agent is an agonist of an OP/BMP morphogen receptor.

In another embodiment the invention provides for a method for delaying the need for, or reducing the frequency of, chronic dialysis treatments, comprising conjointly administering to a mammal: (i) an OP/BMP morphogen, an inducer of endogenous OP/BMP morphogen expression, or an agonist of an OP/BMP morphogen receptor; and (ii) an ACEI.

In another embodiment the invention provides for a method for delaying the need for, or reducing the frequency of, chronic dialysis treatments, comprising conjointly administering to a mammal: (i) an OP/BMP morphogen, an inducer of endogenous OP/BMP morphogen expression or an agonist of an OP/BMP morphogen receptor; and (ii) an AIIRA.

In any of the above mentioned methods of treatment or prevention, said mammal may be afflicted with a condition selected from: chronic renal failure (CRF), end-stage renal disease (ESRD), chronic diabetic nephropathy, diabetic glomerulopathy, diabetic renal hypertrophy, hypertensive nephrosclerosis, hypertensive glomerulosclerosis, chronic glomerulonephritis, hereditary nephritis, or renal dysplasia.

In any of the above mentioned methods of treatment or prevention, the examination of a renal biopsy of said mammal may indicate that said mammal is afflicted with a condition selected from: glomerular hypertrophy, tubular hypertrophy, glomerulosclerosis, or tubulo interstitial sclerosis.

In any of the above mentioned methods of treatment or prevention, the examination of a renal biopsy of said mammal may indicate renal fibrosis. In one embodiment the examination may be by ultrasound, NMR or CAT scan of said mammal.

The invention also comprises use of: (i) an OP/BMP morphogen, an inducer of endogenous OP/BMP morphogen expression, or an agonist of an OP/BMP morphogen receptor; and (ii) an Angiotensin-Converting Enzyme Inhibitor (ACEI) for the preparation of a medicament for treating or preventing chronic renal failure in a mammal.

The invention also comprises use of: (i) an OP/BMP morphogen, an inducer of endogenous OP/BMP morphogen expression, or an agonist of an OP/BMP morphogen receptor; and (ii) an Angiotensin-II Receptor Antagonist (AIIRA) for the preparation of a medicament for treating or preventing chronic renal failure in a mammal.

In another embodiment the invention provides for use of mesenchymal progenitor cells that have been pretreated with an ACEI and an agent that increases the abundance of an OP/BMP morphogen for the preparation of a medicament to be introduced into the kidney of a mammal for treating or preventing chronic renal failure in a mammal.

In another embodiment the invention provides for use of mesenchymal progenitor cells that have been pretreated with an AIIRA and an agent that increases the abundance of an OP/BMP morphogen for the preparation of a medicament to be introduced into the kidney of a mammal for treating or preventing chronic renal failure in a mammal. In one embodiment the agent is an OP/BMP morphogen. In another embodiment the agent is an inducer of an OP/BMP morphogen. In another embodiment the agent is an agonist of an OP/BMP morphogen receptor.

The invention also comprises use of an (i) OP/BMP morphogen, an inducer of endogenous OP/BMP morphogen expression, or an agonist of an OP/BMP morphogen receptor; and (ii) an ACEI to prepare a medicament for delaying or reducing the frequency of chronic dialysis treatments in a mammal.

The invention also comprises use of an (i) OP/BMP morphogen, an inducer of endogenous OP/BMP morphogen expression, or an agonist of an OP/BMP morphogen receptor; and (ii) an AIIRA to prepare a medicament for delaying or reducing the frequency of chronic dialysis treatments in a mammal.

In any of the above mentioned embodiments, said mammal may possess a number of functional nephron units which is less than about 40% of a number of functional nephron units present in a mammal having intact healthy kidneys. In one embodiment, said mammal possesses a number of functional nephron units which is less than about 20% of a number of functional nephron units present in a mammal having intact healthy kidneys.

In any of the above mentioned embodiments, said mammal may be a kidney transplant recipient. In one embodiment, said mammal possesses only one kidney.

In any of the above mentioned embodiments, examination of a urinary sediment of said mammal may indicate a presence of broad casts.

In some of the above mentioned embodiments, said mammal may have a GFR which is chronically less than about 40% of a GFRₑₓₚ for said mammal. In some of the above mentioned embodiments, said mammal may have a GFR which is chronically less than about 20% of a GFRₑₓₚ for said mammal.

In any of the above mentioned embodiments, said mammal may be a human male weighing at least about 50 kg and has a GFR which is chronically less than about 40 ml/min. In any of the above mentioned embodiments, said mammay may be a human female weighing at least about 40 kg and has a GFR which is chronically less than about 30 m/min.

In any of the above mentioned embodiments, said method of treatment or prevention, or said medicament, may reduce reduce serum creatinine levels in said mammal by at least about 5% over 3 months.

In any of the above mentioned embodiments, prior to said treatment or prevention, said mammal may present a chronic decline in a clinical indicator of renal function, and after at least about 3 months of said treatment or prevention, said indicator may stabilize.

In any of the above mentioned embodiments, at least one of said ACEI, said AIIRA or said morphogen may be administered orally, parenterally, intravenously, intraperitoneally, or into a renal capsule, or by an implanted device. In any of the above mentioned embodiments, a stent may be implanted into said mammal for said administration of at least one of said ACEI, said AIIRA or said morphogen.

In any of the above mentioned embodiments, at least one of said ACEI or said AIIRA, and at least one of said morphogen may be conjointly administered at least once a week for a period of at least about one month.

In any of the above mentioned embodiments, at least one of said ACEI or AIIRA, and at least one of said morphogen may be conjointly administered at least once a week for a period of at least about one year.

In any of the above mentioned embodiments, said ACEI or said AIIRA, and said morphogen may be administered: (i) through different routes or (ii) at different frequencies.

In any of the above mentioned embodiments, said morphogen may be administered at a dosage of about 0.01-1000 μg/kg body weight of said mammal.

In any of the above mentioned embodiments, said morphogen may be administered at a dosage of a dosage of about 10-300 μg/kg body weight of said mammal.

In any of the above mentioned embodiments that comprises the administration or use of ACEI, said ACEI may be administered orally at a concentration of about 1-10,000 mg/L, preferably 10-1000 mg/L, 10-100 mg/L, 100-1000 mg/L, most preferably 100 mg/L.

In any of the above mentioned embodiments that comprises the administration or use of AIIRA, said AIIRA may be administered orally at a concentration of about 0.01-100 mg/kg body weight, preferably 0.1-10 mg/kg body weight, 0.2-5 mg/kg body weight, 0.5-2 mg/kg body weight, most preferably 1 mg/kg body weight.

In any of the above mentioned embodiments, said OP/BMP morphogen and, ACEI or AIIRA may be administered in a single pharmaceutical composition. In any of the above mentioned embodiments, said OP/BMP morphogen and, ACEI or AIIRA may be administered in separate pharmaceutical compositions at or around the same time. In any of the above mentioned embodiments, said OP/BMP morphogen and, ACEI or AIIRA may be administered in separate pharmaceutical compositions at different times.

In any of the above mentioned embodiments, said morphogen may: (a) induce chondrogenesis in an ectopic bone assay; (b) prevent, inhibit, delay or alleviate loss of renal function in an animal model of chronic renal failure, or (c) cause a clinically significant improvement in a standard marker of renal function when administered to a mammal in, or at risk of, chronic renal failure.

In any of the above mentioned embodiments, said morphogen may comprise a polypeptide including at least a C-terminal cysteine domain of a protein selected from: a pro form, a mature form, or a soluble form of a polypeptide, wherein said polypeptide is: OP-1, OP-2, OP-3, BMP2, BMP3, BMP4, BMP5, BMP6, or BMP9.

In any of the above mentioned embodiments, said morphogen may comprise a polypeptide including at least a C-terminal cysteine domain of a polypeptide selected from: a pro form, a mature form, or a soluble form of human OP-1.

In one embodiment, the morphogen used in any of the above mentioned embodiments may comprise a polypeptide having at least 70% homology or 50% identity with an amino acid sequence of a C-terminal seven-cysteine domain of human OP-1 (SEQ ID NO: 2). In another embodiment, the morphogen used in any of the above mentioned embodiments may comprise a polypeptide having at least 75% homology or 60% identity with an amino acid sequence of a C-terminal. seven-cysteine domain of human OP-1 (SEQ ID NO: 2). In another embodiment, the morphogen used in any of the above mentioned embodiments may comprise a polypeptide having at least 80% homology or 70% identity with an amino acid sequence of a C-terminal seven-cysteine domain of human OP-1 (SEQ ID NO: 2). In another embodiment, the morphogen used in any of the above mentioned embodiments may comprise a polypeptide having at least 90% identity with an amino acid sequence of a C-terminal seven-cysteine domain of human OP-1 (SEQ ID NO: 2).

In any of the above mentioned embodiments said ACEI may be: any one compound of the formulas I-XXVIII or their salts thereof; acylmercapto and mercaptoalkanoyl prolines; captopril (1-[(2S)-3-mercapto-2-methylpropionyl]-L-proline); ether or thioether mercaptoacyl prolines; zofenopril; carboxyalkyl dipeptides; enalapril (N-(1-ethoxycarbonyl-3-phenylpropyl)-L-ananyl-L-proline); lisinopril; quinapril; ramipril; carboxyalkyl dipeptide mimics; cilazapril; benazapril; phosphinylalkanoyl prolines; fosinopril; trandolopril; phosphonamidate substituted amino or imino acids; phosphonate substituted amino or imino acids and salts thereof; ceronapril ((S)-1-[6-amino-2-[[hydroxy](4-phenylbutyl)phosphinyl]oxy]-1-oxohexyl]-L-proline); BRL 36,378; MC-838; CGS 14824 (3-([1-ethoxycarbonyl-3-phenyl-(1S)-propyl]-amino)-2,3,4,5-tetrahydro-2-oxo-1-(3S)-benzazepine-1 acetic acid HCL); CGS 16,617 (3(S)-[[(1S)-5-amino-1-carboxypentyl]amino]2,3,4,5-tetrahydro-2-oxo-1H-1-benzazepine-1-ethanoic acid); Cetapril (alacepril, Dainippon); Ru 44570; Cilazapril; Ro 31-2201; Lisinopril; Indalapril (delapril); Rentiapril (fentiapril, Santen); Indolapril; Spirapril; Perindopril; Quinapril; CI 925 ([3S-[2[R(*)R(*)]]3R(*)]-2-[2-[[1-(ethoxy-carbonyl)-3-phenylpropyl]amino[-1-oxopropyl]-1,2,3,4-tetrahydro-6,7-dimethoxy-3-isoquinolinecarboxylic acid HCL); WY-44221; mercapto-containing compounds; pivopril; YS980; Omapatrilat; Alacepril; moveltopril; quinaprilat; moexipril; perinodpril (S-9490); pentopril; ancovenin; phenacein; or nicotianamin. In a preferred embodiment the ACEI is Enalapril.

In any of the above mentioned embodiments said AIIRA may be: Losartan (Cozaar®), Valsartan (Diovan®), Irbesartan (Avapro®), Candesartan (Atacand®), Telmisartan (Micardis®), tasosartan, zolarsartan, Teveten (eprosartan mesylate) or olmesartan medoxomil (Benicar).

ACE inhibitors, AIIRAs and/or morphogens may be formulated with one or more pharmaceutically acceptable carriers (additives) and/or diluents. As described in detail below, such pharmaceutical compositions may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intrathecal, intracerebroventricular, intramuscular, or intravenous injection as, for example, a sterile solution or suspension, including administration using a minipump or other mechanical-assisted delivery, such as ALZET osmotic pumps that continuously deliver agents at controlled rates; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile water. In certain embodiments, the pharmaceutical preparation is non-pyrogenic, i.e., does not elevate the body temperature of a patient.

The phrase ‘therapeutically effective amount’ as used herein means that amount of a compound, material, or composition comprising a compound of the present invention which is effective for producing some desired therapeutic effect in at least a sub-population of cells in an animal and thereby blocking the biological consequences of that pathway in the treated cells, at a reasonable benefit/risk ratio applicable to any medical treatment.

The phrase ‘pharmaceutically acceptable’ is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase ‘pharmaceutically acceptable carrier’ as used herein means a pharmaceutically acceptable material, composition or vehicle, such as a liquid or solid filler, diluent, excipient, solvent or encapsulating material, involved in carrying or transporting the subject compounds from one organ, or portion of the body, to another organ, or portion of the body. Each carrier must be ‘acceptable’ in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain compounds contain a basic functional group, such as amino or alkylamino, and are, thus, capable of forming pharmaceutically acceptable salts with pharmaceutically acceptable acids. The term ‘pharmaceutically acceptable salts’ in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, compounds contain one or more acidic functional groups and, thus, are capable of forming pharmaceutically acceptable salts with pharmaceutically acceptable bases. The term ‘pharmaceutically acceptable salts’ in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Morphogens, morphogen inducers, or agonists of morphogen receptors, as well as ACE inhibitors (ACEIs) may be administered by any route which is compatible with the particular morphogen, inducer, agonist, or ACEI employed. Thus, as appropriate, administration may be oral or parenteral, including intravenous and intraperitoneal routes of administration. In addition, administration may be by periodic injections of a bolus of the morphogen, inducer, agonist or ACEI, or may be made more continuous by intravenous or intraperitoneal administration from a reservoir which is external (e.g., an ix. bag) or internal (e.g., a bioerodable implant, or a colony of implanted, morphogen-producing cells).

Therapeutic agents of the invention (i.e., morphogens, morphogen inducers, agonists of morphogen receptors, or ACEI) may be provided to an individual by any suitable means, directly (e.g., locally, as by injection, implantation or topical administration to a tissue locus) or systemically (e.g., parenterally or orally). Where the agent is to be provided parenterally, such as by intravenous, subcutaneous, intramolecular, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the agent preferably comprises part of an aqueous or physiologically compatible fluid suspension or solution. Thus, the carrier or vehicle for the agent(s) is physiologically acceptable so that in addition to delivery of the desired agent(s) to the patient, it does not otherwise adversely affect the patient's electrolyte and/or volume balance. The fluid medium for the agent thus can comprise normal physiologic saline (e.g., 9.85% aqueous NaCl, 0.15 M, pH 7-7.4).

Association of the mature morphogen dimer with a morphogen pro domain results in the pro form of the morphogen which typically is more soluble in physiological solutions than the corresponding mature form. In fact, endogenous morphogens are thought to be transported (e.g., secreted and circulated) in the mammalian body in this form. This soluble form of the protein can be obtained from culture medium of morphogen-secreting mammalian cells, e.g., cells transfected with nucleic acid encoding and competent to express the morphogen. Alternatively, a soluble species can be formulated by complexing the mature, morphogenically active polypeptide dimer (or an active fragment thereof) with a morphogen pro domain polypeptide or a solubility-enhancing fragment thereof. Solubility-enhancing pro domain fragments can be any N-terminal, C-terminal or internal fragment of the pro region of a member of the morphogen family that complexes with the mature polypeptide dimer to enhance stability and/or dissolubility of the resulting noncovalent or covalent complex. Typically, useful fragments are those cleaved at the proteolytic site Arg-Xaa-Xaa-Arg (SEQ ID NO: 30). A detailed description of soluble complex forms of morphogenic proteins, including how to make, test and use them, is described in WO 94/03600 (PCT US 93/07189). In the case of OP-1, useful pro domain polypeptide fragments include the intact pro domain polypeptide (residues 30-292) and fragments 48-292 and 158-292, all of SEQ ID No. 3. Another molecule capable of enhancing solubility and particularly useful for oral administrations, is casein. For example, addition of 0.2% casein increases solubility of the mature active form of OP-1 by 80%. Other components found in milk and/or various serum proteins may also be useful.

Useful solutions for parenteral administration may be prepared by any of the methods well known in the pharmaceutical art, described, for example, in REMMINGTON'S PHARMACEUTICAL SCIENCES (Gennaro, A., ed.), Mack Pub., 1990. Formulations of the therapeutic agents of the invention may include, for example, polyalkylene glycols such as polyethylene glycol, oils of vegetable origin, hydrogenated naphthalenes, and the like. Formulations for direct administration, in particular, may include glycerol and other compositions of high viscosity to help maintain the agent at the desired locus. Biocompatible, preferably bioresorbable, polymers, including, for example, hyaluronic acid, collagen, tricalcium phosphate, polybutyrate, lactide, and glycolide polymers and lactide/glycolide copolymers, may be useful excipients to control the release of the agent in vivo. Other potentially useful parenteral delivery systems for these agents include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene 9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or citric acid for vaginal administration. Suppositories for rectal administration may also be prepared by mixing the morphogen, inducer or agonist with a non-irritating excipient such as cocoa butter or other compositions which are solid at room temperature and liquid at body temperatures.

Formulations for topical administration to the skin surface may be prepared by dispersing the morphogen, inducer, agonist, or ACEI with a dermatologically acceptable carrier such as a lotion, cream, ointment or soap. Particularly useful are carriers capable of forming a film or layer over the skin to localize application and inhibit removal. For topical administration to internal tissue surfaces, the agent may be dispersed in a liquid tissue adhesive or other substance known to enhance adsorption to a tissue surface. For example, hydroxypropylcellulose or fibrinogen/thrombin solutions may be used to advantage. Alternatively, tissue-coating solutions, such as pectin-containing formulations may be used.

Alternatively, the agents described herein may be administered orally. Oral administration of proteins as therapeutics generally is not practiced, as most proteins are readily degraded by digestive enzymes and acids in the mammalian digestive system before they can be absorbed into the bloodstream. However, the morphogens described herein typically are acid stable and protease-resistant (see, for example, U.S. Pat. No. 4,968,590). In addition, at least one morphogen, OP-1, has been identified in mammary gland extract, colostrum and 57-day milk. Moreover, the OP-1 purified from mammary gland extract is morphogenically active and is also detected in the bloodstream. Maternal administration, via ingested milk, may be a natural delivery route of TGF-β superfamily proteins. Letterio, et al., Science 264: 1936-1938 (1994), report that TGF-β is present in murine milk, and that radio-labeled TGF-β is absorbed by gastrointestinal mucosa of suckling juveniles.

Labeled, ingested TGF-β appears rapidly in intact form in the juveniles' body tissues, including lung, heart and liver. Finally, soluble form morphogen, e.g., mature morphogen associated with the pro domain, is morphogenically active. These findings, as well as those disclosed in the examples below, indicate that oral and parenteral administration are viable means for administering TGF-β superfamily proteins, including the morphogens, to an individual. In addition, while the mature forms of certain morphogens described herein typically are sparingly soluble, the morphogen form found in milk (and mammary gland extract and colostrum) is readily soluble, probably by association of the mature, morphogenically active form with part or all of the pro domain of the expressed, full length polypeptide sequence and/or by association with one or more milk components. Accordingly, the compounds provided herein may also be associated with molecules capable of enhancing their solubility in vitro or in vivo.

Most ACE inhibitors can be orally administered. In a preferred embodiment, ACE inhibitor as a pharmaceutical composition can be orally administered through drinking water or other suitable liquid carrier.

The compounds provided herein may also be associated with molecules capable of targeting the morphogen, inducer, agonist, or ACEI to the desired tissue. For example, an antibody, antibody fragment, or other binding protein that interacts specifically with a surface molecule on cells of the desired tissue, may be used.

Useful targeting molecules may be designed, for example, using the single chain binding site technology disclosed in U.S. Pat. No. 5,091,513. Targeting molecules can be covalently or non-covalently associated with the morphogen, inducer, agonist, or ACEI.

As will be appreciated by one of ordinary skill in the art, the formulated compositions contain therapeutically effective amounts of the morphogen, morphogen inducers, agonists of morphogen receptors, or ACEI. That is, they contain an amount which provides appropriate concentrations of the agent to the affected tissue for a time sufficient to stimulate a detectable restoration of impaired renal system function, up to and including a complete restoration thereof. As will be appreciated by those skilled in the art, these concentrations will vary depending upon a number of factors, including the biological efficacy of the selected agent, the chemical characteristics (e.g., hydrophobicity) of the specific agent, the formulation thereof, including a mixture with one or more excipients, the administration route, and the treatment envisioned, including whether the active ingredient will be administered directly into a tissue site, or whether it will be administered systemically. The preferred dosage to be administered is also likely to depend on variables such as the condition of the diseased or damaged tissues, and the overall health status of the particular mammal. As a general matter, single, daily, biweekly or weekly dosages of 0.00001-1000 mg of a morphogen are sufficient, with 0.0001-100 mg being preferable, and 0.001 to 10 mg being even more preferable. Alternatively, a single, daily, biweekly or weekly dosage of about 0.01-1000 μg/kg body weight, more preferably about 0.01-10 μg/kg body weight, or about 10-300 μg/kg body weight may be advantageously employed. As a general matter, single, daily, biweekly, or weekly dosages of ACEI can be administered orally at an amount of about 0.01-300 mg/kg body weight, preferably 0.1-30 mg/kg BW, 0.1-3 mg/kg BW, 1-30 mg/kg BW, most preferably about 1-3 mg/kg BW, in, for example, drinking water, are appropriate for ACE inhibitors. The concentrations can be accordingly adjusted or alternatively expressed as the amount of drug that needs to be administered per day per kg of body weight, if other factors (such as the average body weight of a subject mammalian patient being treated, and the average amount of water consumed per day by said specific mammalian patient) are provided. The present effective dose can be administered in a single dose or in a plurality (two or more) of installment doses, as desired or considered appropriate under the specific circumstances. A bolus injection or diffusible infusion formulation can be used. If desired to facilitate repeated or frequent infusions, implantation of a semi-permanent stent (e.g., intravenous, intraperitoneal, intracisternal or intracapsular) may be advisable. It should also be understood that the dosages of morphogens and/or ACEIs, when conjointly administered, may be different from the dosages of morphogens or ACEIs when they are administered alone (not conjoint administration). It should also be understood that a particular dosage of morphogen or ACEI for treating/preventing chronic renal failure may be different from dosages used for other non-related (such as bone morphogenesis, etc.) uses of the morphogens and/or ACEIs.

The morphogens, inducers, agonists, or ACEI of the invention may, of course, be administered alone or in combination with other molecules known to be beneficial in the treatment of the conditions described herein. For example, various well-known growth factors, hormones, enzymes, therapeutic compositions, antibiotics, or other bioactive agents can also be administered with the morphogen and ACEI. Thus, various known growth factors such as NGF, EGF, PDGF, IGF, FGF, TGF-α, and TGF-β, as well as enzymes, enzyme inhibitors, antioxidants, anti-inflammatory agents, free radical scavenging agents, antibiotics and/or chemoattractant/chemotactic factors, can be included in the present morphogen and ACEI formulation.

Finally, it should be understood that morphogen formulation and ACEI formulations of the present invention can be either formulated together, in a single pharmaceutical composition, or formulated separately, in two or more pharmaceutical compositions. It should also be understood that the same formulation can be administered through different routes, depending on specific needs or appropriate treatment conditions.

C. Types of Chronic Renal Failures

Many types of Chronic Renal Failure diseases can be treated according to the instant invention. The following discussions are for illustration purpose only, and should not be construed to be limiting in any respect.

The present invention is directed to methods of prevention and/or treatment, and pharmaceutical preparations for use in the prevention and/or treatment, of vertebrate subjects (preferably mammalian subjects) in, or at risk of, chronic renal failure, or at risk of the need for renal replacement therapy. Such subjects include subjects already afflicted with chronic renal failure, or which have already received renal replacement therapy, as well as any subject reasonably expected to suffer a progressive loss of renal function associated with progressive loss of functioning nephron units. Whether a particular subject is at risk is a determination which may routinely be made by one of ordinary skill in the relevant medical or veterinary art. Subjects in, or at risk of, chronic renal failure, or at risk of the need for renal replacement therapy, include but are not limited to the following: subjects which may be regarded as afflicted with chronic renal failure (CRF), end-stage renal disease (ESRD), chronic diabetic nephropathy, diabetic glomerulopathy, diabetic renal hypertrophy, hypertensive nephrosclerosis, hypertensive glomerulosclerosis, chronic glomerulonephritis, hereditary nephritis, or renal dysplasia; subjects having a biopsy indicating glomerular hypertrophy, tubular hypertrophy, chronic glomerulosclerosis, and/or chronic tubulointerstitial sclerosis; subjects having an-ultrasound, MRI, CAT scan, or other non-invasive examination indicating renal fibrosis; subject shaving an unusual number of broad casts present in urinary sediment; subjects having a GFR which is chronically less than about 50%, and more particularly less than about 40%, 30% or 20%, of the expected GFR for the subject; human male subjects weighing at least about 50 kg and having a GFR which is chronically less than about 50 ml/min, and more particularly less than about 40 ml/min, 30 ml/min or 20 ml/min; human female subjects weighing at least about 40 kg and having a GFR which is chronically less than about 40 mL/min, and more particularly less than about 30 mL/min, 20 ml/min or 10 m/min; subjects possessing a number of functional nephron units which is less than about 50%, and more particularly less than about 40%, 30% or 20%, of the number of functional nephron units possessed by a healthy but otherwise similar subject; subjects which have a single kidney; and subjects which are kidney transplant recipients.

Chronic renal failure (CRF) can be classified by the site (location) of primary damage: Pre-renal CRF, Post-renal CRF and Renal CRF.

Pre-Renal CRF. Some medical conditions cause continuous hypoperfusion (low blood flow) of the kidneys, leading to kidney atrophy (shrinking), loss of nephron function, and chronic renal failure (CRF). These conditions include poor cardiac function, chronic liver failure, and atherosclerosis (“hardening”) of the renal arteries. Each of these conditions can induce ischemic nephropathy, which is a result of inadequate blood flow (hypoperfusion) to the kidneys. Hypoperfusion manifests as a progressive loss of kidney function and kidney atrophy (shrinkage). Renal failure results when this process damages both kidneys.

Post-Renal CRF. Interference with the normal flow of urine can produce backpressure within the kidneys, can damage nephrons, and lead to obstructive uropathy, a disease of the urinary tract. Abnormalities that may hamper urine flow and cause post-renal CRF include the following:
Bladder outlet obstruction due to an enlarged prostate gland or bladder stone;
Neurogenic bladder, an over distended bladder caused by impaired communicator nerve fibers from the bladder to the spinal cord;
Kidney stones in both ureters, the tubes that pass urine from each kidney to the bladder;
Obstruction of the tubules, the end channels of the renal nephrons;
Retroperitoneal fibrosis, the formation of fiber-like tissue behind the peritoneum, the membrane that lines the abdominal cavity;
Vesicoureteral reflux (VUR), the backward flow of urine from the bladder into a ureter.

Renal CRF. Chronic renal failure caused by changes within the kidneys, is called renal CRF, and is broadly categorized as follows: Diabetic nephropathy, kidney disease associated with diabetes; the most common cause of CRF;
Hypertension Nephrosclerosis, a condition that occurs with increased frequency in African Americans; the second leading cause of CRF;
Chronic glomerular nephritis, a condition caused by diseases that affect the glomeruli and bring about progressive dysfunction;
Chronic interstitial nephritis, a condition caused by disorders that ultimately lead to progressive scarring of the interstitium;
Renal vascular CRF, large vessel abnormalities such as renal artery stenosis (narrowing of the large arteries that supply the kidneys);
Vasculitis, inflammation of the small blood vessels;
Cystic kidney disease, kidney disease distinguished by multiple cysts (lined cavities or sacs);
Hereditary diseases of the kidney, such as Alport's syndrome (hereditary nephritis).

More detailed descriptions of a few of the above conditions are provided below.

Diabetic nephropathy is kidney disease that develops as a result of diabetes mellitus (DM). DM, also called simply diabetes, affects approximately 5% of the U.S. population. This disease damages many organs, including the eyes, nerves, blood vessels, heart, and kidneys. DM is the most common cause of kidney failure in the United States and accounts for over one-third of all patients who are on dialysis.

DM patients are unable to metabolize carbohydrates (e.g., food starches, sugars, cellulose) properly. The disease is characterized by excessive amounts of sugar in the blood (hyperglycemia) and urine; inadequate production and/or utilization of insulin; and by thirst, hunger, and loss of weight.

Diabetics who require daily insulin shots to maintain life have insulin-dependent diabetes mellitus, or DM 1. In this type of diabetes, the pancreas β cells secrete little or no insulin and the blood sugar level remains high, unless treated. DM 1 usually occurs in children and young adults and is often called juvenile onset diabetes. Onset of the disease is abrupt. The patient becomes very sick and requires immediate insulin therapy. Approximately 1 million people in the United States have DM 1.

Approximately 25% to 40% of patients with DM 1 ultimately develop diabetic nephropathy (DN), which progresses through about five predictable stages.

During Stage 1 (very early diabetes), increased demand upon the kidneys is indicated by an above-normal glomerular filtration rate (GFR). During Stage 2 (developing diabetes), the GFR remains elevated or has returned to normal, but glomerular damage has progressed to significant microalbuminuria (small but above-normal level of the protein albumin in the urine). Patients in stage 2 excrete more than 30 mg of albumin in the urine over a 24-hour period. Significant microalbuminuria will progress to end-stage renal disease (ESRD). Therefore, all diabetes patients should be screened for microalbuminuria on a routine (yearly) basis. During Stage 3 (overt, or dipstick-positive diabetes), glomerular damage has progressed to clinical albuminuria. The urine is “dipstick positive,” containing more than 300 mg of albumin in a 24-hour period. Hypertension (high blood pressure) typically develops during stage 3. During Stage 4 (late-stage diabetes), glomerular damage continues, with increasing amounts of protein albumin in the urine. The kidneys' filtering ability has begun to decline steadily, and blood urea nitrogen (BUN) and creatinine (Cr) has begun to increase. The glomerular filtration rate (GFR) decreases about 10% annually. Almost all patients have hypertension at stage 4. During Stage 5 (end-stage renal disease, ESRD), GFR has fallen to approximately 10 milliliters per minute (<10 mL/min) and renal replacement therapy (i.e., hemodialysis, peritoneal dialysis, kidney transplantation) is needed. Progression through these five stages is rather predictable because the onset of DM 1 can be identified, and most patients are free from age-related medical problems.

Non-insulin-dependent diabetes, or DM 2, differs from DM 1 in that the main problem is a peripheral resistance to the action of the insulin. DM 2 usually occurs in adults over the age of 40 who are overweight and have a family history of the disease. Some patients can manage their diabetes with weight loss and changes in their diet. Others require medication, and many with DM 2 eventually require insulin. Onset is gradual, and patients may be sick for quite some time without knowing it. Nearly 95% of diabetics are diagnosed with DM 2. An estimated 5% to 15% of DM 2 cases also progress through the five stages of diabetic nephropathy (DN), but the timeline is not as clear. Some patients advance through the stages very quickly.

Renal artery stenosis (RAS) is the narrowing of the lining of the main artery that supplies the kidney. Most RAS is caused by atherosclerosis or “hardening of the arteries.” Atherosclerosis is the build up of cholesterol deposits, or plaque, in the lining of the arteries. Depending on the degree of narrowing, patients can develop hypertension called renal vascular hypertension (RVH). This form of hypertension is the most common cause of secondary hypertension. In fact, hypertension is second only to diabetes as the leading cause of kidney failure. In the US, 15%-20% of kidney failure cases are due to hypertension. RVH occurs when RAS produces a critical narrowing of the artery that supplies one of the kidneys. Critical RAS is defined as at least 70% narrowing of the renal artery, based on angiographic (blood vessel x-ray) evaluation. Reduced blood flow through the renal artery causes the kidney to release increased amounts of the hormone renin. Renin, a powerful blood pressure regulator, initiates a series of chemical events that result in hypertension. Renal vascular hypertension can be very severe and difficult to control.

The kidney with RAS suffers from the decreased blood flow and often shrinks in size (atrophies). This process is called ischemic nephropathy. The other kidney is at risk for developing damage from the hypertension, often developing hypertensive nephrosclerosis. The persistent elevated blood pressures in this non-stenotic kidney can cause progressive scarring (sclerosis) leading to progressive loss of filtering function in this kidney as well. Both unilateral RAS and bilateral RAS can ultimately lead to chronic renal failure.

There are two types of RAS: Atherosclerotic Renal Artery Stenosis (AS-RAS) and Fibromuscular Dysplasia (FMD). AS-RAS is due to the build-up of cholesterol on the inner lining of the renal artery. It is exceedingly more common then the unusual case of FMD-RAS. FMD-RAS occurs almost exclusively in women aged 30 to 40 and rarely affects African Americans or Asians. FMD-RAS is due to an abnormality in the muscular lining of the renal artery. There is often a familial history of FMD RAS.

Cystic kidney disease describes several conditions in which fluid-filled cysts form in the kidneys. Cysts generally develop in weak segments of the tubules that carry urine from the glomeruli. The cyst's growth displaces healthy kidney tissue. The kidneys expand to accommodate the cyst, which can weigh as much as 20 pounds. Three factors determine cyst classification: its cause (acquired, inherited), its features (complicated, simple, multiple, single), and its location (outer [cortical] or inner [medullary] kidney tissue).

Polycystic kidney disease (PKD; common, with several cysts in the kidney) is a primary cystic kidney disease. PKD type 1 and PKD type 2 are caused by autosomal dominant mutations on chromosomes 16 and 4, respectively, and run in families. PKD autosomal recessive has been linked to chromosome 6. Polycystic kidney disease (PKD) is the most frequently inherited disease; it affects approximately 600,000 people in the United States and over 12,000,000 worldwide. Most suffer from the autosomal dominant type. It is the fourth leading cause of kidney failure and causes 10% of all end-stage renal disease (ESRD), usually between the ages of 40 and 60. It affects men, women, and races equally.

Secondary cystic kidney disease include Acquired cystic kidney disease (ACKD); Medullary cystic disease (inner kidney), which includes Juvenile nephronophthisis (during adolescence) and Medullary sponge kidney (deterioration of kidney with cysts); and Renal cell cancer associated cysts.

Autosomal dominant medullary cystic kidney disease (MCK) causes cysts to form in the inner tissue of the kidney and can develop at a very early age. Recessive juvenile nephronophthisis usually occurs later than MCK, but is associated with similar symptoms, including chronic renal failure and growth problems. Small cysts in the collecting ducts of the inner kidney characterize medullary sponge kidney (MSK), which is associated with hematuria and kidney stones, but not chronic renal failure (CFR).

Acquired cystic kidney disease (ACKD) affects patients with chronic renal failure and causes hematuria, erythrocytosis (increase in red blood cells), and is associated with the development of cancer. Causes of acquired cystic kidney disease (ACKD) are long-term disease (glomerulonephritis) and the scarring that often results from dialysis. ACKD is common among patients with chronic renal failure (CRF). Nearly all of those who use dialysis for more than 5 years develop ACKD.

Proteinuria is an abnormally high amount of protein in the urine. Proteins in the blood, like albumin and immunoglobulin, help coagulation (clotting), balance bodily fluids, and fight infection. The kidneys remove wastes from protein-rich blood through millions of tiny filtering screens called glomeruli. Most proteins are too large to pass through the glomeruli into the urine. The glomeruli are negatively charged, so they repel the negatively charged proteins. Thus, a size and charge barrier keeps protein molecules from entering the urine. But when the glomeruli are damaged, proteins of various sizes pass through them and are excreted in the urine.

The following five types of proteinuria are distinguished by milligrams (mg) of protein measured during a 24-hour urine collection:

| | | |
|---|---|---|
| 1. Microalbuminuria | 30-150 | mg |
| 2. Mild | 150-500 | mg |
| 3. Moderate | 500-1000 | mg |
| 4. Heavy | 1000-3000 | mg |
| 5. Nephrotic range | more than 3500 | mg |

As kidney disease progresses, more protein enters the urine. People with nephrotic-range proteinuria typically have extensive glomeruli damage and usually develop nephrotic syndrome (see below).

Hypertension and diabetes are the two biggest risk factors for proteinuria. Old age and weight gain also increase the risk. The following conditions cause proteinuria:
Acute glomerulonephritis;
Amyloidosis (protein deposits associated with chronic disease);
Focal glomerulonephritis;
Hypertension;
IgA nephropathy;
Mesangial proliferation
Minimal change disease;

Foamy urine and swelling (edema) are two signs of proteinuria that become more evident as the disease progresses. Excess protein can cause the urine to foam in water. This occurs because protein changes the surface tension between urine and water. Edema usually only occurs in nephrotic range proteinuria.

Albumin is particularly useful in absorbing bodily fluid into the blood. Because the albumin molecule is relatively small, it is often among the first proteins to enter the urine after glomeruli are damaged. Therefore, even minor kidney dysfunction is detectable with proper diagnosis of micoralbuminuria. Reduced albumin level in the blood causes fluid retention and swelling that is first noticeable in the hands, lower legs, and feet. In more serious cases, the abdomen and face may swell.

Orthostatic proteinuria is a disorder seen occasionally in children and young adults who leak significant amounts of urine when they are upright (orthostatic). Presumably, standing increases the pressure on the glomeruli and causes more protein to enter the urine, while lying down relieves pressure and causes less protein leakage. This is a benign disorder that most young people outgrow.

Hypertensive people who develop proteinuria stand a significant chance for kidney failure. African Americans are 20 times more likely than Caucasians to develop hypertensive-related kidney failure. Proteinuria in people with diabetes may be a sign that kidney disease is worsening. Microalbuminuria is often cited as a risk for coronary artery disease (CAD) and is often diagnostic of it and related cardiovascular conditions.

Nephrotic syndrome (NS) is a condition that is often caused by any of a group of diseases that damage the kidneys' filtering system, the glomeruli. The structure of the glomeruli prevents most protein from getting filtered through into the urine. Normally, a person loses less than 150 mg of protein in the urine in a 24-hour period. Nephrotic-range proteinuria, the urination of more than 3.5 grams of protein during a 24-hour period, or 25 times the normal amount, is the primary indicator of NS.

About two in every 10,000 people experience nephrotic syndrome. Nephrotic syndrome prevalence is difficult to establish in adults because the condition is usually a result of an underlying disease. In children, it is diagnosed in more boys than girls, usually between 2 and 3 years of age.

In addition to proteinuria, there are three main symptoms of nephrotic syndrome associated with protein leaking into the urine:
Hypoalbuminemia (low level of albumin in the blood);
Edema (swelling);
Hypercholesterolemia (high level of cholesterol in the blood);

Hypoalbuminemia is a low level of albumin (a protein) in the blood due to proteinuria. Low albumin in the blood causes fluid to move from the blood into the tissue, causing swelling. The kidney perceives the decrease of fluid in the blood and aggressively retains as much fluid and salt as it can. This contributes to the body's fluid-overload state.

Nephrotic-related swelling makes tissue puffy, soft, and impressionable to the touch. Edema is most common in the legs and feet, especially after standing all day. It can cause feelings of tightness in the extremities and may affect mobility. In later stages, swelling may occur in the abdomen (ascites), hands, and around the eyes in the morning (called periorbital edema). In later stages, the whole body may swell (anasarca). Some people gain weight after fluid builds up in their bodies for a long time.

Hypercholestrolemia, high blood cholesterol, is common in nephrotic syndrome. In addition to albumin, other important enzymes involved in cholesterol metabolism slip through the glomeruli, which contribute to high blood cholesterol.

Nephrotic syndrome is associated with renal failure. The disease that causes NS can damage the glomeruli and can interfere with their ability to clean the blood. The edema that is present in the legs may also be occurring in the kidney tissue itself and can interfere with the kidneys' ability to clean the blood. Renal failure can either be gradual (CRF) or acute (ARF).

A hypercoaguable state, in which the blood abnormally overclots, is also seen in some patients with NS. This means that they are at risk for developing a blood clot in the legs or in the renal veins that transport blood from the kidney. Some patients take blood thinners to prevent this complication.

There are a number of different disorders that can cause NS. Diabetes and, to a lesser extent, hypertension can cause diffuse damage to the glomeruli and can ultimately lead to NS. The following diseases can cause specific damage to the glomeruli and often result in the development of heavy proteinuria and in many instances NS: Amyloidosis (the stiffening and subsequent malfunction of the kidney due to fibrous protein deposit in the tissue); Congential nephrosis; Focal segmental glomerular sclerosis (FSGS) (creates scar tissue in the glomerulus, damaging its protein-repellant membrane); Glomerulonephritis (GN), including Diffuse mesangial proliferative GN (affecting the messangium), Membranous (damages the protein-repellant membrane), and Post infectious (occurs after an infection); IgA nephropathy (Berger's disease) (deposit of specific immunoglobulin A causing an inflammatory reaction and leading to glomerulonephritis); Minimal change disease (Nil's disease); and Pre-eclampsia (rarely associated with NS, more often associated with heavy proteinuria).

Many of these diseases tend to occur more often in certain age groups. Less than 1 yr old: Congenital nephrosis. Less than 15 years old: Min change, FSGS, and Other. Age 15 to 40: Min change, FSGS, and Other. Over age 40: Membranous GN, and Diabetic nephropathy. Over 60: Amyloidosis may account for up to 20% of cases.

In addition, physical injury to the kidneys (loss of one kidney plus damage to the other, etc.) and other equivalent situations, such as complete or partial loss of kidney function due to diseases affecting the total number of functional nephrons (filtering units that consist of a glomerulus and corresponding tubule), may cause the remaining functional nephrons to “attempt” compensating for the renal damage by hyperfiltration (excessive straining of the blood). Over time, hyperfiltration causes further loss of renal function, leading to chronic renal failure.

### IV. EXEMPLIFICATION

Practice of the invention, including additional preferred aspects and embodiments thereof, will be still more fully understood from the following examples, which are presented herein for illustration only and should not be construed as limiting the invention in any way.

For the following experiments, “OP-1” is used interchangably with “sBMP-7.”

### Example 1

### Nephrectomy Chronic Renal Failure Injury Model

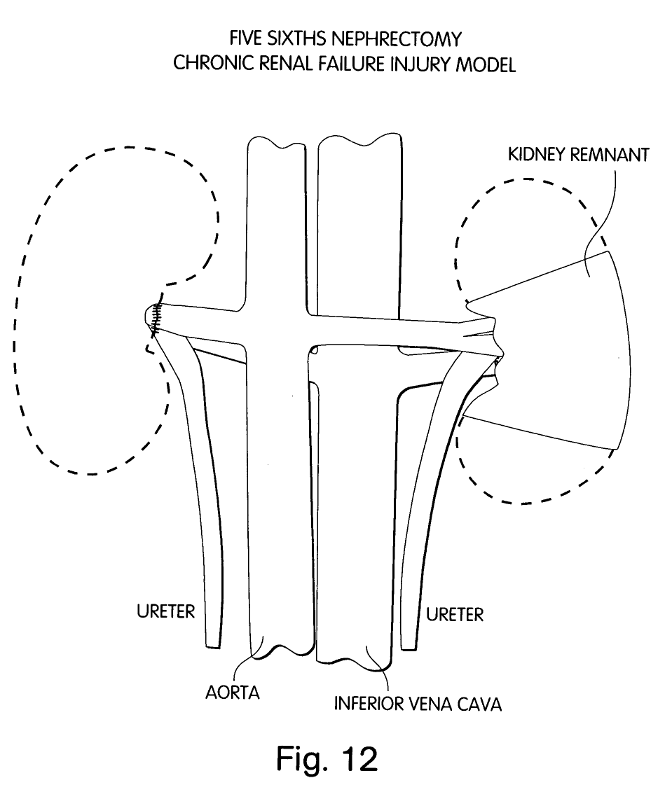

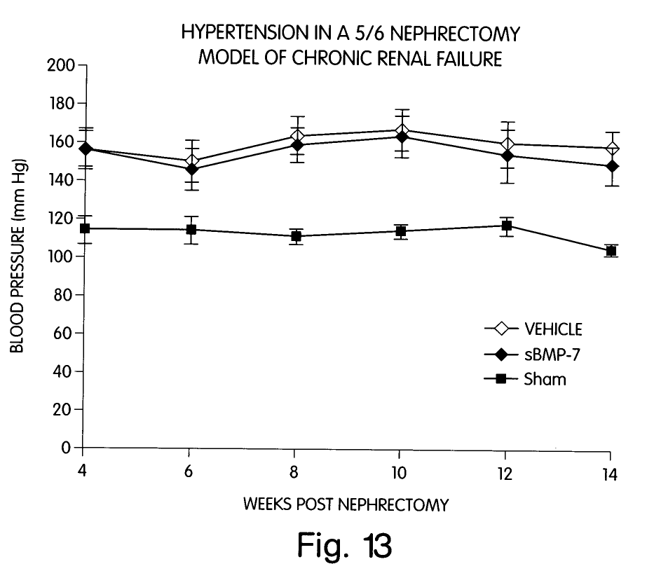

A rat partial (5/6) nephrectomy or rat remnant kidney model (RRKM) model was employed essentially as described (Vukicevic, et al. (1987) J. Bone Mineral Res. 2: 533, the entire content of which is incorporated herein by reference). Male Munich-Wistar rats (2-3 months old, weighing about 200-250 g) were subjected to renal mass ablation. Specifically, the right kidney is excised in conjunction with selective ligation of the left renal artery branches such that one third of the kidney remains perfused (5/6 NPX, see FIG. 12). Immediately following surgery, plasma creatinine and BUN levels rise dramatically due to the loss of renal mass and function. Over the next several weeks of this “acute” failure phase, plasma creatinine and BUN levels of surviving animals decline somewhat toward normal values but remain elevated. Renal function then appears to remain relatively constant or stable for a period of variable duration. After this point, the animals enter a period of chronic renal failure in which there is an essentially linear decline in renal function ending in death. For these reasons, animals are given 4 weeks of recovery prior to initiation of treatments.

Four weeks after nephrectomy, rats were divided into four treatment groups (see Table III), namely OP-1 (150 μg/kg body weight, 3×/week, by i.p. injection), Vehicle (20 mM arginine/150 mM NaCl, 0.1% Tween-80, pH 9.0, 1 ml/kg body weight, 3×/week, by i.p. injection), enalapril (100 mg/L in drinking water, 8-16 mg/kg body weight), and OP-1 with enalapril. For the first two groups (OP-1 and Vehicle), animals were sacrificed 14 weeks after nephrectomy. For the last two groups (enalapril, and OP-1 with enalapril), animals were sacrificed 26 weeks after nephrectomy. As surgical controls, rats were subjected to a “sham” operation in which the kidneys were decapsulated but no renal tissue was removed. Sham-treated animals were sacrificed 26 weeks after operation. None of the rats died in any group during this study. Systolic blood pressure, urine protein and/or glomerulosclerosis were monitored at intervals between 4 and 26 weeks post surgery.

TABLE III
Experimental design in the Nephrectomy Chronic Renal Failure
Injury Model.

| Group | Animals (N) | Treatment | Duration |
|---|---|---|---|
| I | 5 | Sham | 26 weeks |
| II | 10 | OP-1 | Initiate treatment 4 weeks |
| III | 10 | Vehicle | after nephrectomy |
| | | | Terminate animals 14 |
| | | | weeks after nephrectomy |
| IV | 10 | OP-1 + enalapril | Initiate treatment 4 |
| V | 10 | Vehicle + enalapril | weeks after nephrectomy |
| | | | Terminate animals 26 |
| | | | weeks after nephrectomy |

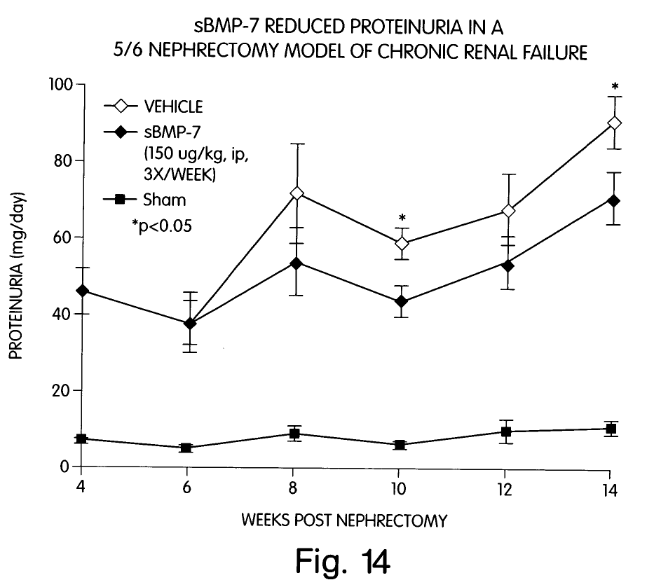

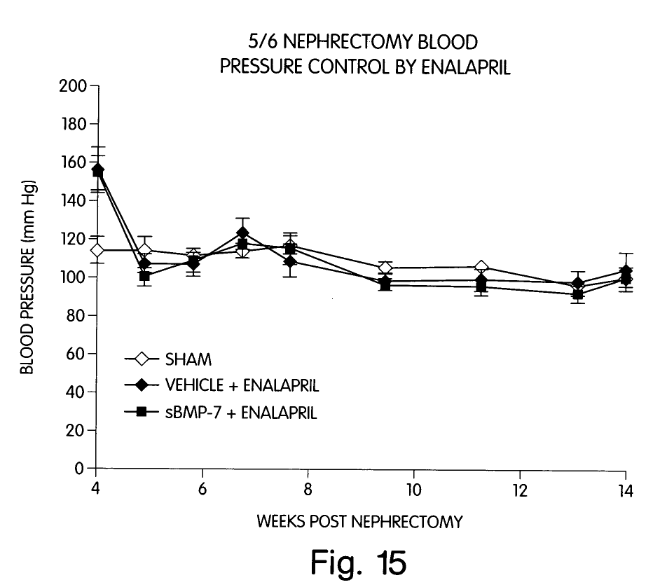

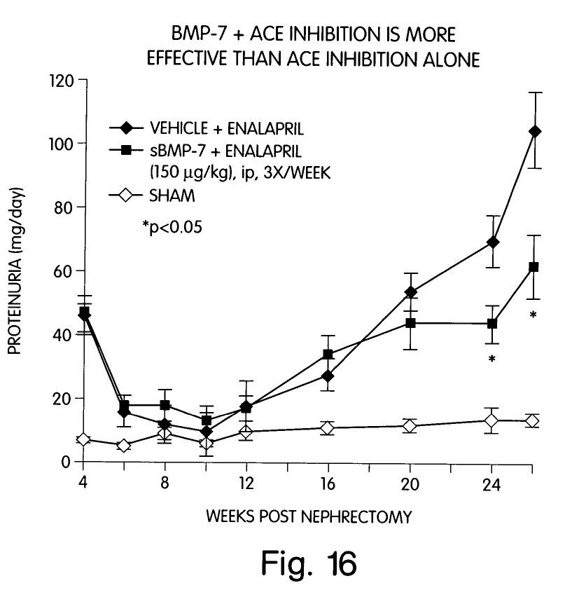

As compared to sham-operated controls, animals after Nephrectomy exhibited symptoms of higher blood pressure and higher proteinuria. While OP-1 did not dramatically lower the blood pressure level (see FIG. 13), OP-1 significantly reduced the proteinuria level, from 91 mg/day to 71 mg/day (P<0.05), after 14 weeks post-Nephrectomy (see FIG. 14). When animals were co-treated with OP-1 and enalapril, there was no additional benefit in reducing the blood pressure of nephrectomized animals to normal level as compared to animals treated by the ACE inhibitor enalapril alone (see FIG. 15). However, the combination of OP-1 and enalapril was more effective in reducing the proteinuria level than enalapril alone, 62 mg/day vs. 105 mg/day (see FIG. 16).

In summary, the results from the Nephrectomy Chronic Renal Failure Injury Model demonstrate that OP-1 improves glomerular filtration rate, reduces glomerulosclerosis, and reduces proteinuria. Co-treatment of OP-1 and enalapril reduces late-stage proteinuria more than enalapril alone, thus may have a better effect on renal functions.

### Example 2

### Unilateral Ureteral Obstruction (UUO) Renal Fibrosis Model

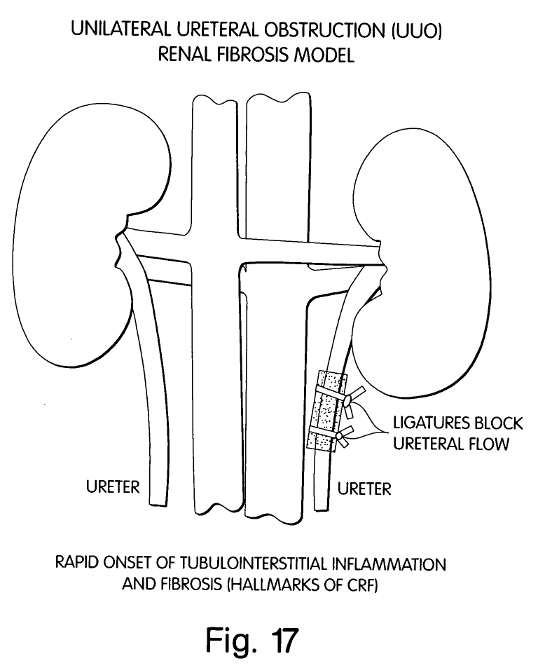

This UUO model was employed essentially as described (Moller, et al. (1984) Virchows Arch 402: 209-237, the entire contents of which are incorporated herein by reference). Sprague-Dawley rats (about 250 g) underwent either a sham operation (ureter manipulated but not ligated) or unilateral ureteral ligation. Two ligatures, 5 mm apart, were placed in the upper two-thirds of the ureter over a section of polyethylene tubing placed around the ureter (see FIG. 17). The suture tied to obstruct the ureter was removed along with the tubing at day 5, relieving the obstruction. In this model, hypertension, proteinuria, and lipid dysregulation do not contribute to progressive nephron destruction, and glomerular injury is not prominent early in the course of the injury produced. Uremia is avoided by the function of the contralateral kidney, which undergoes hypertrophy and hyperplasia as the obstructed kidney is destroyed. The renal injury of UUO is mediated in part through stimulation of renal angiotensin II production, which activates type-1 A-II Receptor and the downsteam TGF-β in a cascade of events culminating in tubulointerstitial inflammation and fibrosis. Inhibition of angiotensin II production by ACE inhibitors (or inhibition of A-II receptors) decreases expansion of the renal interstitium associated with fibrosis.

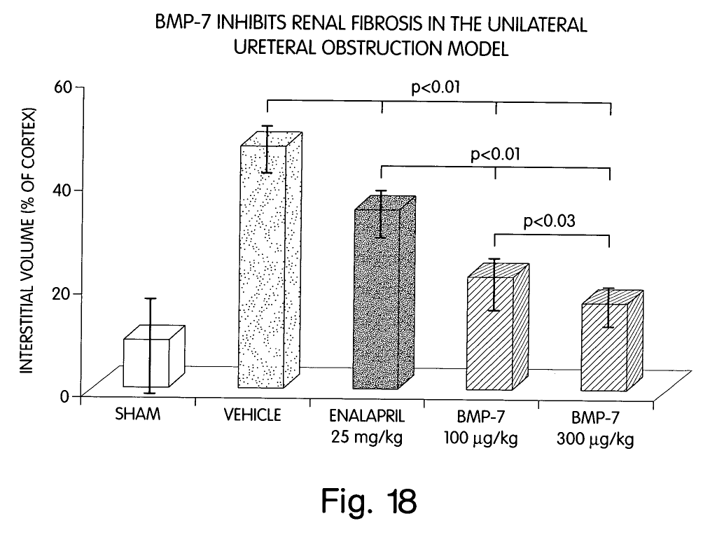

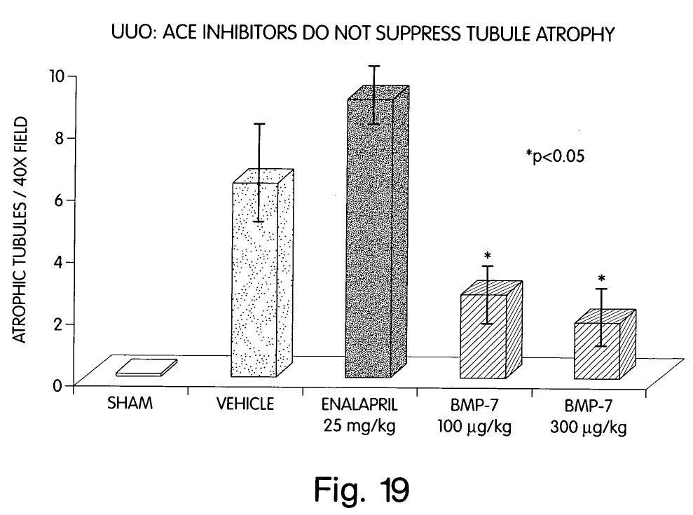

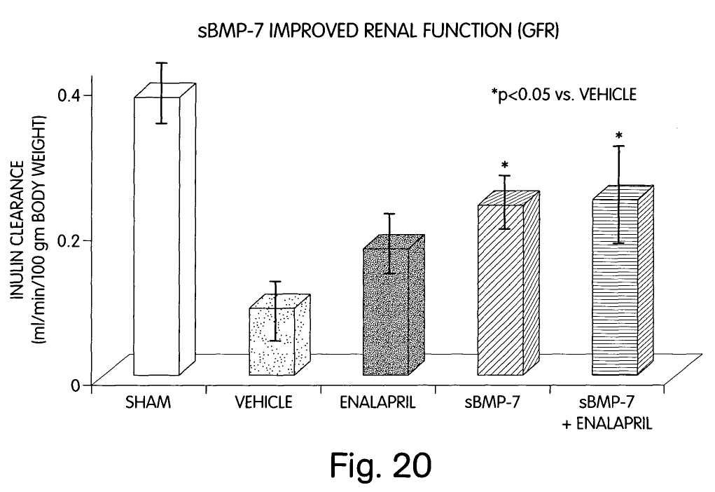

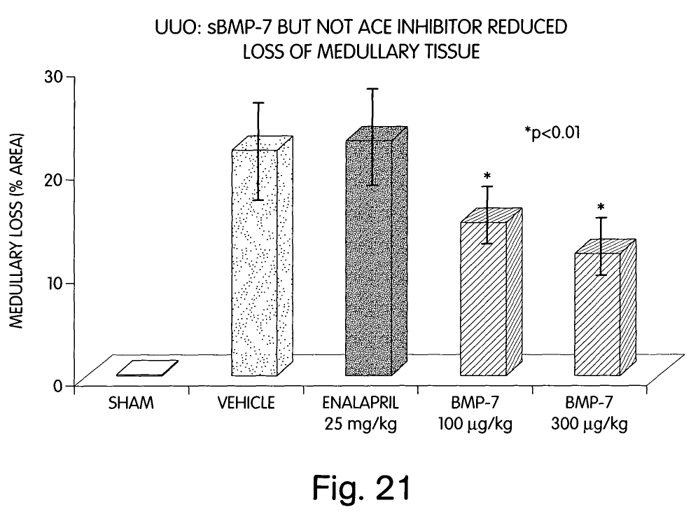

The effect of OP-1 on Renal fibrosis has been published (Hruska, et al. (2000) Am J Physiol Renal Physiol 279:F130-43, incorporated herein by reference), and a summarization is as follows. Administration of OP-1 (100 or 300 μg/kg body weight) prevented interstitial inflammation and fibrogenesis during the first 5 days after obstruction. Compared with ACE inhibition (by enalapril treatment), OP-1 was more effective in preventing tubulointerstitial fibrosis and in preserving renal function (see FIG. 18). The mechanism of OP-1-induced renal protection was associated with prevention of tubular atrophy, an effect not shared with enalapril (see FIG. 19). OP-1 blocked the stimulation of epithelial cell apoptosis produced by UUO, which promoted maintenance of tubular epithelial integrity. OP-1 preserved renal blood flow (RBF) during UUO, but enalapril also stimulated RBF. OP-1 was more efficacious than enalapril in improving the glomerular filtration rate as evidenced by the inulin clearance rate (see FIG. 20). OP-1 also inhibited tubular epithelial disruption stimulated by the renal injury of UUO. Additional effects of OP-1 have been observed in this rat UUO model. For example, OP-1, but not ACE inhibitor, significantly reduced the loss of medullary tissue in the kidney (see FIG. 21), from about 24% to about 16% (OP-1 at 100 μg/kg) or about 13% (OP-1 at 300 μg/kg) (P<0.01).

In summary, the results from the UUO Renal Fibrosis Model demonstrate that OP-1 reduces renal fibrosis, increases the glomerular filtration rate, and reduces tubular atrophy and medullary necrosis. OP-1 is more effective than enalapril. However, the combination of OP-1 and enalapril may have additive benefits on renal functions.

### Example 3

### Streptozotocin-Induced Diabetic Nephropathy Model

Nephropathy is one of the most common and most serious complications in type 1 diabetes mellitus. Renal involvement usually starts with renal hypertrophy and glomerular hyperfiltration, which can be observed soon after diabetes onset (Mogensen, et al. (1994) Diabetes Care 17:770-775). Glomerular hyperfiltration is often accompanied by a loss of renal functional reserve. After some years, microalbuminuria (30 to 300 mg/day) may occur as well as morphological changes such as thickening of the glomerular basement membrane and mesangial expansion. The albumin leakage may subsequently become aggravated and overt nephropathy with albuminuria (>300 mg/day) may develop, usually 10 to 20 years after the onset of diabetes. At this time, hypertension becomes more common. Nephrotic syndrome may occur, and glomerular filtration rate declines. The most important therapeutic measures undertaken to avoid, or retard, the progress of nephropathy aim to improve glycemic control and normalize blood pressure. ACE inhibitors have proven effective in the latter respect.

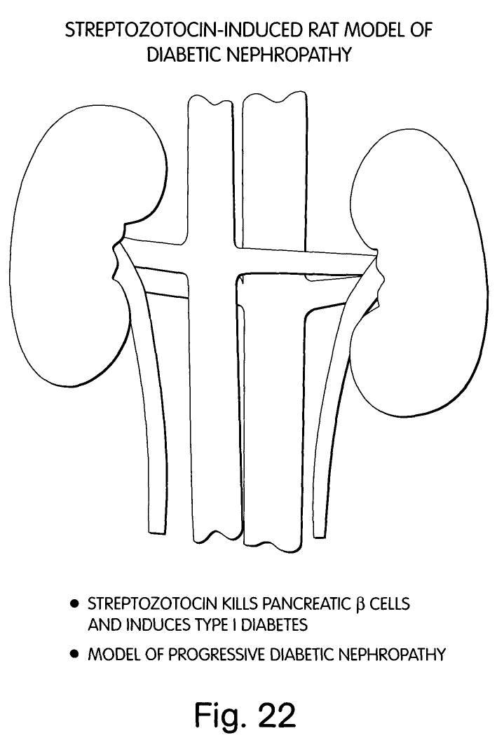

Streptozotocin kills pancreatic β cells and induces type I diabetes (for review, see Cheta et al. (1998) J Pediatr Endocrinol Metab 11:11-9). It is widely used to induce experimental diabetic nephropathy in animals (see FIG. 22). Adult female Sprague-Dawley rats (weighing 200-250 g) were intraperitoneally injected with streptozotocin (60 mg/kg body weight) to induce hyperglycemia. Hyperglycemic rats then received daily injections of insulin to maintain blood glucose between 200-400 mg/dL. At week 16 when renal function declined, animals were treated weekly with OP-1 (10, 30 or 100 μg/kg body weight), enalapril (50 or 100 mg/L in drinking water) or a combination of OP-1 and enalapril. Control animals without streptozotocin treatment were handled in all other ways like treated animals. Animals were sacrificed at week 32 post-streptozotocin treatment.

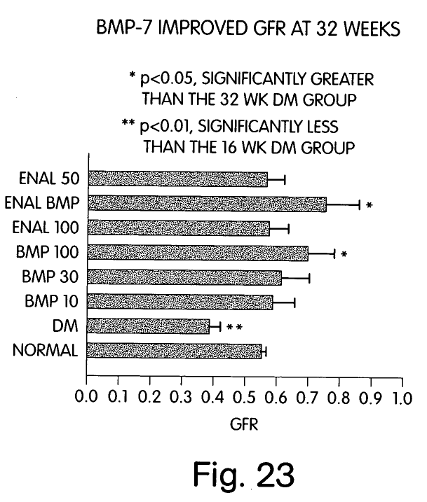

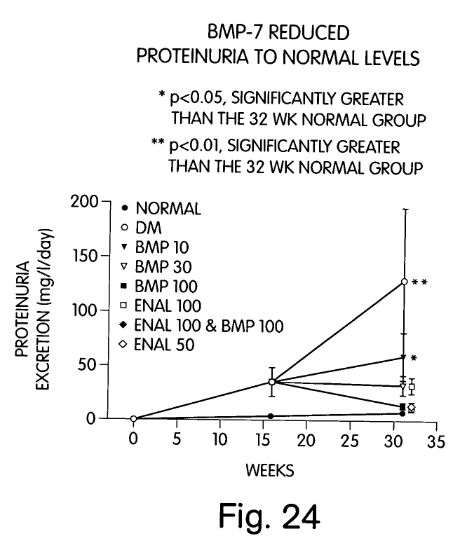

Animals treated with streptozotocin exhibited symptoms of lower glomerular filtration rate and higher proteinuria level. As seen in FIG. 23, OP-1, but not enalapril, significantly increased the glomerular filtration rate (GFR). While the mean GFR in the 32-week diabetic animals was about 0.38 ml/min/100 g body weight, the mean GFR in the animals treated with 100 μg/kg of OP-1 was about 0.7 ml/min/100 g body weight (P<0.05). The mean GFR in the animals co-treated with OP-1 and enalapril was even higher, about 0.75 ml/min/100 g body weight (P<0.05). As seen in FIG. 24, OP-1 (30 and 100 μg/kg), enalapril (50 and 100 mg/L) or the combination of OP-1 and enalapril, significantly reduced the proteinuria level from about 140 mg/day to almost normal level.

In summary, the results from Streptozotocin-Induced Diabetic Nephropathy Model demonstrate that OP-1 increases the glomerular filtration rate and reduces proteinuria. The combinatorial treatment of OP-1 and enalapril may have better effects on renal functions.

### Example 4

### Alloxan-Induced Diabetic Nephropathy Model

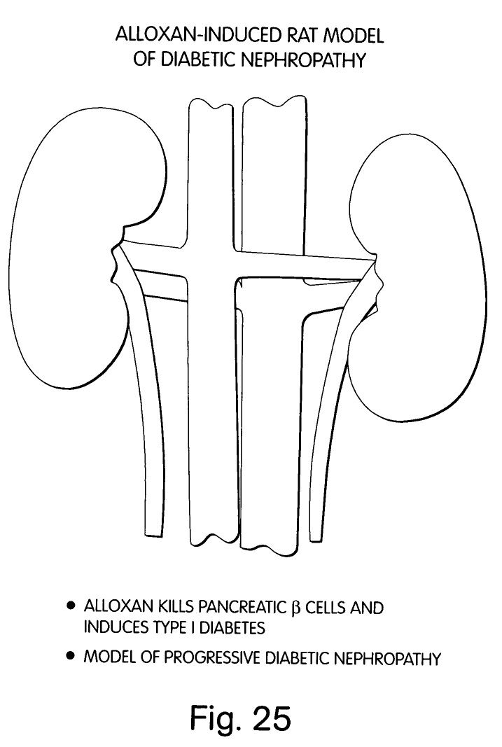

Similar to streptozotocin, alloxan kills pancreatic β cells and induces type I diabetes (for review, see Cheta et al. (1998) J Pediatr Endocrinol Metab 11: 11-9). Alloxan is also widely used to induce experimental diabetic nephropathy in animals (FIG. 25). Adult female Sprague-Dawley rats (weighing 200-250 g) were intraperitoneally injected with alloxan (70 mg/kg body weight) to induce hyperglycemia. Renal arteries were clamped just prior to injection to prevent direct nephrotoxicity, and then clamps were removed 5 minutes after injection. At week 16 when renal function declined, animals were treated with OP-1 (10 μg/kg body weight, 3×/week, or 30 μg/kg body weight, 1×/week), enalapril (100 mg/L in drinking water) or the combination of OP-1 and enalapril. Control animals without alloxan treatment were handled in all other ways like treated animals. The treatment duration was 12 weeks. All rats were sacrificed after 26 weeks.

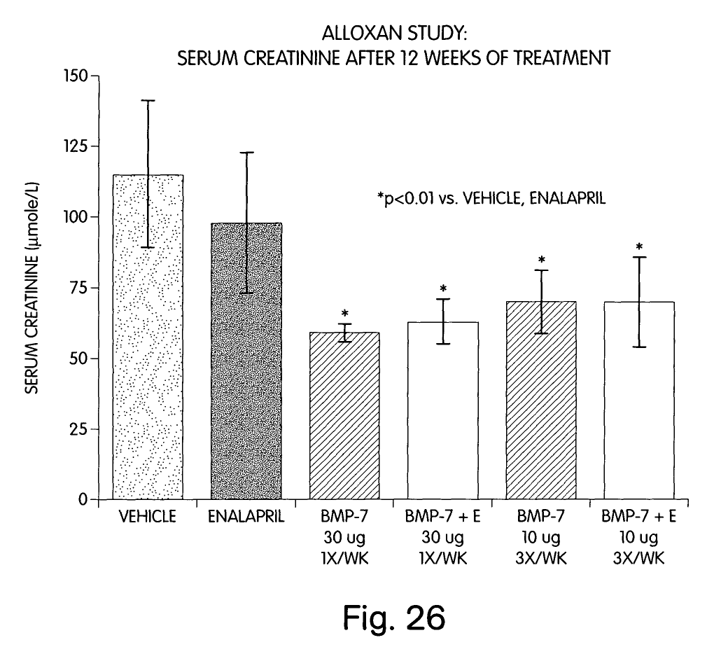

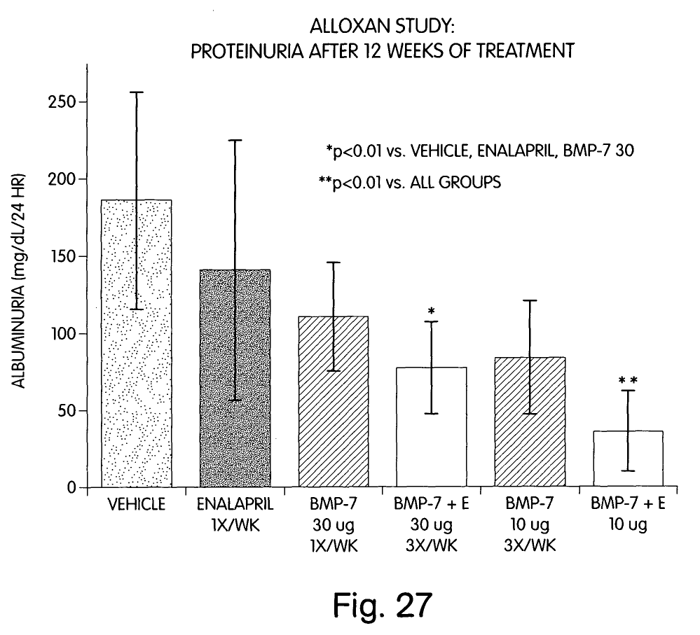

Animals treated with alloxan showed higher serum creatinine level and higher proteinuria after 12-weeks of treatment. As seen in FIG. 26, OP-1 (10 or 30 μg/kg) dramatically reduced the serum creatinine level, from about 115 μmole/L to about 65 μmole/L or 55 μmole/L (P<0.01). Compared with OP-1, elalapril reduced the serum creatinine level at a lesser degree. The combination of OP-1 and elanapril also significantly reduced the serum creatinine level. As seen FIG. 27, OP-1 (10 or 30 μg/kg) or enalapril reduced the proteinuria level, from about 180 mg/dL/24 hr to about 80 (or 110) or 140 mg/dL/24 hr, respectively. In contrast, the combination of OP-1 and elanapril dramaticaly reduced the proteinuria level to as low as about 30 mg/dL/24 hr (P<0.01).

In summary, the results from Alloxan-Induced Diabetic Nephropathy Model demonstrate that OP-1 or enalapril decreases the serum creatinine level and reduces the proteinuria level. The combinatorial treatment of OP-1 and enalapril shows better effects on renal functions.

### Example 4

### Bone Morphogenic Protein-7 (BMP-7), a Novel Effective Therapy for Diabetic Nephropathy

Overview

A long-term streptozotocin model of diabetic nephropathy was used to test and compare the therapeutic actions of BMP-7 with those of Enalapril. The study design was a treatment protocol beginning at 16 weeks when glomerular hypertrophy and proteinuria were established. The effects of therapy with BMP-7 (10, 30, or 100 μg/kg iv, biw) were compared to a maximal dose of Enalapril (20 mg/kg) and to a vehicle control. The highest dose of BMP-7 and Enalapril were equal in partially reversing kidney hypertrophy. In the diabetic rats treated with BMP-7, 100 μg/kg, GFR at 32 weeks was significantly higher than in the diabetic vehicle treated rats, 0.7±0.08 vs 0.34±0.02 ml/min/100 g bw (P<0.05). The GFR of 32 week diabetic Enalapril treated rats was 0.58±0.06 (not significant compared to vehicle treated and sham injected rats 0.55±0.02). Albuminuria was reversed to normal by BMP-7 in a dose dependent manner.

The reduction in proteinuria by the intermediate dose of BMP-7 was similar to the effect of Enalapril therapy. Glomerular area and interstitial volume were significantly decreased in the BMP-7 and Enalapril treated animals. Glomerular sclerosis was prevented by BMP-7 therapy more effectively than by Enalapril. The first insight into the mechanism of BMP-7 therapy, was produced by analysis of blood pressures. Enalapril controlled hypertension throughout the course of therapy while BMP-7 did not effect blood pressure until the final four weeks of therapy. Important mechanistic insight derived from the demonstration that lost epithelial cell differentiation marked by loss at hyperglycemic vehicle treated diabetic rats BMP-7 expression in the kidney. At the same time another developmental morphogen, Wnt4, was widely expressed. BMP-7 and Enalapril therapy restored BMP-7 expression at high levels in the collecting duct without affecting Wnt4 expression. We conclude that BMP-7 reversed diabetic and hyperglycemia induced glomerular hypertrophy and injury, restoring GFR, protein excretion in glomerular histology towards normal and generally outperforming Enalapril. Restoration of BMP-7 expression, representing preservation of the collecting duct phenotype, restored the normal developmental Wnt4 interacting partner. This was associated with a successful repair reaction and a reversal of the ill-fated injury response.

Introduction

Recent studies have made the surprising observation that a renal tubular developmental morphogen, bone morphogenetic protein-7 (BMP-7), was effective in preventing the tubulointerstitial nephritis stimulated by obstructive uropathy (1). The mechanism of action appeared to be preservation of epithelial cell phenotype, inhibition of epithelial-mesenchymal transdifferentiation and inhibition of injury induced epithelial cell apoptosis. These actions of BMP-7 are reminiscent of the effects that the morphogen exercises during development.

During vertebrate development, the permanent kidney is generated by the interactions of the ureteric bud and the metanephric mesenchyme (2,3). At day 11 post-coitum (dpc) in the mouse, the ureteric bud branches out and invades the metanephric mesenchyme. Thereafter, nephrogenesis derives from reciprocal inductive interaction between these two tissues. The metanephric mesenchyme induces the ureteric bud to grow and bifurcate to form the collecting ducts. At the same time permissive survival signals from the ureteric bud interact with mesenchymal signals to induce the conversion of metanephric mesenchyme into an epithelial structure. Epithelialization begins at day 11.5 pc with condensation around the ureteric bud, and it progresses with the condensed mesenchyme segregating into pretubular aggregates. Epithelialization of these aggregates leads to development of the comma shaped bodies, S-shaped bodies and eventually the epithelial component of the nephron including glomerular podocytes.

BMP-7 has been shown to be a required factor leading to the condensation and epithelialization of the metanephric mesenchyme, and the reciprocal induction of collecting duct differentiation (4-6). It is expressed in the ureteric bud, and in the condensing mesenchyme at day 11.5. BMP-7 is a survival factor for the condensing mesenchymal cells which die between 12.5 and 14.5 dpc in its absence (4). At day 12 pc, in the absence of BMP-7, glomerulus formation ceases as the mesenchymal cells apoptose. BMP-7 interacts with another critical tubular inductive morphogen, Wnt4 (7)(8). Wnt4 expression is initiated at day 12.5 in the aggregating mesenchyme and pretubular aggregates. It is required as an inductive signal for epithelization (7). At birth, BMP-7 deficient kidneys are dysgenic, hypoplastic and cystic with severely dilated collecting ducts separated by areas of stromal cells an extracellular matrix. The kidneys are hydronephrotic, and they do not have metanephric mesenchyme or evidence of glomerulus formation in the cortical nephrogenic zone (4,5). Cysts appear to originate from derivatives of the ureteric bud suggesting abnormal activity of the cell cycle and disordered polarity in these cells. Glomerular density is less than 3/section compared to greater than 100/section in wild type kidneys (8). Wnt4 deficient kidneys, on the other hand are also dysplastic and hydronephrotic, but they are totally devoid of glomeruli (8).

Compared to many other morphogens, expression of BMP-7 in the tubular epithelial segments derived from the ureteric bud does not cease following its developmental inductive actions, rather its expression persists and it likely functions physiologically as a collecting duct epithelial cell differentiation factor. As such it inhibits proliferation by blocking progress of the cell cycle at the G1 checkpoint and it prevents apoptosis (1,9).

Tissue injuries frequently stimulate attempts at repair that recapitulate development, including entry of surviving tissue cells into the cell cycle. This would require the actions of differentiation factors to be covercome perhaps by decreasing their levels. In turn, the absence of key factors could cause these attempted repairs to default into a fibrotic process resulting in permanent loss of the starting tissue. Unsuccessful injury repair characterizes many renal diseases that lead to loss of excretory function. Recently, renal injuries, including that caused by high glucose levels, have been shown to decrease BMP-7 levels (1,10-12). Treatment of one of these injuries with BMP-7 prevented renal tubular atrophy and epithelial cell apoptosis. Failure of tubular development and mesenchymal apoptosis are features of development in the BMP-7 deficient state.

Here we report in a proof of concept study that BMP-7 is an effective therapy for diabetic nephropathy. We report the discovery of a new mechanism of renal injury stimulated by diabetes, that of re-expression of a critical tubular epithelial inductive signal, Wnt4, that interacts with BMP-7 during development. Wnt4 is known to stimulate tubulointerstitial fibrosis, and its re-expression during diabetic hyperglycemic injury is a mechanism of synergism with TGF® resulting in a failed injury repair reaction and promotion of disease. Therapy of diabetic nephropathy with BMP-7 partially reversed the renal injury induced by diabetes and hyperglycemia, and it prevented the development of glomerulosclerosis. The actions of BMP-7 were compared to the known therapeutic agent for diabetic nephropathy—angiotensin converting enzyme inhibition. While both agents were efficacious, BMP-7 was more effective in reversing proteinuria and preventing glomerulosclerosis. Diabetic injury resulted in the loss of tubular epithelial and glomerular podocyte phenotype manifested by loss of BMP-7 expression, and therapy with BMP-7 restored the phenotype of the collecting duct manifested by restoration of BMP-7 expression. BMP-7, in turn, stimulated a successful repair reaction possibly by interacting with Wnt4 and inhibiting the actions of TGF®.

Materials and Methods

Animals: Female Sprague-Dawley rats 10 weeks of age weighing 190-220 g were used. Animals were allowed free access to standard rat chow and to tap water. Diabetes mellitus was induced by a single tail-vein injection of Streptozotocin (STZ 62 mg/kg of body weight, Sigma chemical company, St. Louis, Mo.) dissolved in normal saline at day 0. The diabetic state was confirmed 72 hours later by the determination of blood glucose concentration>300 mg/dl. From day 3, all the diabetic rats received daily subcutaneous injections of 1.0-7.0 u human recombinant long-acting insulin injection (Eli Lilly & Co., Indianapolis, Ind.) as required to maintain the blood glucose concentration between 300-500 mg/dl. Tail blood glucose levels were measured twice a week with an Accu—Chek™ Advantage meter (Roche Diagnostic Corporation, Indianapolis, Ind.). Body weights were taken once a week. Food and water intake were monitored daily.

Renal hypertrophy was well developed at 16 weeks prior to treatment with vehicle, BMP-7, or enalapril. At 1.6 weeks, animals were divided into 8 groups. Group 1 were 16 weeks of DM. Group 2 were 16 weeks normal controls. Group 3 DM animals were treated with tail-vein injection of vehicle twice a week for 16 weeks. Group 4, Group 5, and Group 6 were DM animals treated with tail-vein injections of BMP-7 100, 30, and 10 μg/kg body weight respectively twice a week for 16 weeks. Group 7 were treated with Enalapril 20 mg/kg through the drinking water for 16 weeks. Group 8 animals were normal control showed for 32 weeks alongside the diabetic animals. Group 1 and Group 2 animals were sacrificed at 16 weeks. The others were sacrificed at 32 weeks. All treatments began at 16 weeks and continued through 32 weeks. Glomerular filtration rate (GFR), urine albumin excretion, kidney weight, glomerular area, mesangial matrix area, interstitial volume, monocyte and macrophage infiltration, thickness of glomerular basement membrane (GBM) and glomerulosclerosis were measured.

Renal Function: In all animals, glomerular filtration rate (GFR) was measured as the clearance of inulin. Rats were anesthetized with a Ketamine/Xylozine cocktail. A catheter was inserted into the femoral vein under a dissecting microscope for infusion. Another catheter was placed into the femoral artery for collecting blood samples. Urine was collected by bladder cannulation.

After the completion of surgery, a bolus of 2 ml/kg of 3% inulin (Cypros Pharmaceutical Corp, W. Carlsbad, Calif.) and 0.2% p-aminohippurate (PAH) in normal saline was infused as a priming load, followed by a sustaining infusion of the same solution at the rate of 8 ml/h·kg. Urine collection was initiated for three 20-minute collection periods after an hour of equilibration. An arterial blood sample was obtained during each clearance period. Plasma and urine were analyzed for inulin.

Urine Protein Excretion: Before the clearance study, the rats were placed in individual metabolic cages for two 24 hour urine collections. Urine volume was measured and urinary protein concentration was determined with a Bio-Rad protein assay.

Preparation of Kidneys: After the clearance studies, the rats were euthanized. Both kidneys were rapidly removed and placed in ice-cold phosphate buffered saline (PBS). Kidneys were weighed and then sliced on a cold glass plate. Two 2 mm coronal sections were immersed in Histochoice and in 10% buffered formalin. Kidney sections were embedded in paraffin and cut at 3 μm and stained with hematoxylin and eosin, Gomori's Trichrome and periodic acid Schiff (PAS).

Renal Morphology: The morphometric analysis was done in a blind manner. Osteomeasure™ was used for morphometric analysis. Glomeruli were traced at ×400 magnification. Tissue sections stained by PAS were used. The measured glomerular parameters were as follows: (a) glomerular area, determined out of 60 glomeruli per group, which had vascular pole on it from randomly selected sections. (b) mesangial matrix area (defined as PAS-positive material in the mesangium), (c) ratio of the mesangial matrix area to the glomerular area, and (d) focal segmental glomerulosclerosis (glomerular sclerosis was defined as global sclerosis or segments of glomerular tufts demonstrating collapsed, obliterated capillaries with sparseness of normal cellular elements). The percent of sclerotic glomeruli was determined was determined out of 150 glomeruli per animal from randomly selected sections (13).

Interstitial volume was determined by a point-counting technique on tissue sections stained by the Gomori's Trichrome method, and was expressed as the mean percentage of grid points laying within the interstitial area in up to 5 fields in the cortex (1)(14).

Quantitation of monocyte/macrophage infiltration was determined by counting ED-1 antigen positive cells in tissue sections (1)(14). Kidneys were fixed in Histochoice, paraffin embedded, and sectioned. Sections were dewaxed and rehydrated prior to incubation with the ED-1 antibody obtained from Harlan (Indianapolis, Ind.). The location of the primary antibodies was visualized using alkaline phosphatase-linked second antibody. To obtain numbers of infiltrating monocyte/macrophage in the glomeruli, 50 consecutive cross-sections of glomeruli of each animal were evaluated. To obtain numbers of infiltrating monocyte/macrophage in the renal cortical tubulointerstitium, 10 consecutive non-overlapping fields were counted in each section and viewed at 400× magnification.

Electron microscopy: Fresh tissue was fixed in 3% (wt/vol) glutaraldehyde buffer and post-fixed in OsO₄. Tissue was then dehydrated in ethanol and embedded in Poly/bed 812 resin (Poly Science Inc, Warrington, Pa.). Thin sections were stained with uranyl acetate and lead citrate and examined using a transmission electron microscope.

Blood pressure determinations: The tail artery cuff method was used to follow blood pressures.

In situ hybridizations: ³⁵S-UPT labeled sense and antisense constructs were prepared as previously described (15). Briefly frozen sections (4-6 microns) were fixed in 4% formaldehyde in PBA for 20 min at room temperature. Sections were washed once in PBS at three times the normal concentration of salt. Sections were then washed three times in PBS for 5 min and in water for 2 min and once in 0.1 M triethanolamine, pH 8.0 for 10 min. Additional washings for 10 min in 0.25% acetic anhydride in 0.1 M triethanolamine, pH 8.0 and twice for 2 min in 2×andard sodium citrate were performed. The sections were then dehydrated through graded ethanols followed by air drying for 5 min. This was followed by vacuum drying for 1 h at room temperature. Each slide was then hybridized for 18 h at 60° C. with 106 counts/min of ³³P radiolabeled riboprobe in 80 μl of hybridization mixture (50% formamide; 2% Denhardt's solution; 10% dextran sulfate; 0.3 M NaCl; 10 mM Tris, pH=8.0; 2 mM EDTA, 0.25 g/ml tRNA). The sense and antisense probes for BMP-7 were a kind gift of Kuber Sampath. The sense and antisense probes for Wnt4 were previously described (15). Slides were then treated for 20 min in 4×SSC at room temperature; 4×SSC for 5 min at room temperature; Rnase A (20 μg/ml) for 30 min at 37° C. in 0.5 m NaCl, 0.01 M Tris, pH=8.0 and 1 mM EDTA; twice 2×SSC for 5 min at room temperature; 1×SSC for 10 min at room temperature; 0.5×SSC for 10 min at room temperature; and 0.1×SSC for 30 min at 60° C. The sections were then dehydrated using increasing concentrations of ethanol followed by vacuum desiccation for 30 min. Slides were dipped in liquid photographic emulsion (Kodak NTB2) and exposed for 1 wk at 4° C. After development, slides were counterstained with hematoxylin and eosin.

Statistical Analysis: Results were expressed as mean±SEM. In situ method statistical Analysis was carried out by using a one-way ANOVA, or a nonparametric ANOVA. Statistical significance was achieved if the p<0.05. Data were analyzed using the InStat software.

Results

General Group Comparisons: The number of animals in each group, body weight at the beginning and the study end, kidney weight, blood glucose concentration, insulin dose, urine protein excretion, GFR and the mortality data of each group are summarized in Table 1. There were no significant differences in body weight gain between the different groups during the 16 weeks of treatment. Also there were no significant differences in blood glucose levels and insulin doses between the different groups during the 16 weeks of treatment. The blood glucose levels were significantly higher in the 16 week DM group than those of all the 32 weeks treatment groups, and insulin doses in the 16 week DM were significantly lower than those of all the 32 weeks treatment groups (p<0.01). We increased the insulin doses during the study to increase blood sugar control, in order to decrease the mortality we were encountering. Specifically, the blood sugar range was decreased from 400-600 to 300-500. As can be seen from Table 1, these actions were equally applied to all groups. The mortality observed in the various treatment groups was similar. The causes of death were hypoglycemia, ketoacidosis, and anesthesia. Some animals died of unknown reasons because the biochemical data on the day of death were lacking, and autopsies were not informative, suggesting ketoacidosis or hypoglycemia as the causes.

TABLE 1
Characterization of Animals in Various Groups

| | | Body Weight (g) | | | kidney | Blood | Insulin | Protein |
|---|---|---|---|---|---|---|---|---|
| | | | | Weight | weight | sugar | Dose | Ex |
| Group | N | Beginning | End | Gain | (g) | (mg/dl) | (u/wk) | (mg/day) |
| NI 16 | 7 | | 266.7 ± 6.7 | | 0.81 ± 0.02 | | | 3.76 ± 0.39 |
| NI 32 | 14 | | 296.1 ± 5.1 | | 0.85 ± 0.02 | | | 8.24 ± 1.28 |
| DM 16 | 4 | | | | 1.42 ± 0.02 | 508.1 ± 5.7^{a} | 8 ± 1^{a} | 35.63 ± 13.35 |
| DM 32 | 18 | 219.8 ± 6.3 | 302.1 ± 6.0 | 82.3 ± 8.8 | 1.44 ± 0.04 | 472.9 ± 5.2 | 11 ± 1 | 174.44 ± 52.50 |
| BMP 10 | 9 | 213.4 ± 4.1 | 292.6 ± 7.0 | 79.2 ± 5.5 | 1.19 ± 0.03 | 460.0 ± 5.5 | 12 ± 1 | 59.46 ± 21.84 |
| BMP 30 | 9 | 219.1 ± 3.6 | 280.2 ± 5.9 | 65.6 ± 3.6 | 1.19 ± 0.03 | 455.8 ± 5.7 | 13 ± 1 | 33.02 ± 9.11 |
| BMP 100 | 10 | 204.8 ± 4.9 | 290.3 ± 6.6 | 85.5 ± 6.9 | 1.10 ± 0.03 | 461.6 ± 5.5 | 16 ± 1 | 14.27 ± 3.50 |
| E 100 | 10 | 216.1 ± 4.0 | 294.9 ± 4.4 | 78.7 ± 5.9 | 1.09 ± 0.04 | 459.7 ± 5.9 | 14 ± 1 | 33.59 ± 7.58 |

| | GFR | | | | | |
|---|---|---|---|---|---|---|
| | (ml/min/ | | | Cause-Mortality | | |
| Group | 100 g bw) | Mortality | ketoacidosis | hypoglycemia | anesthesia | unknown |
| NI 16 | 0.49 ± 0.04 | 0/7 | | | | |
| NI 32 | 0.55 ± 0.02 | 0/14 | | | | |
| DM 16 | 1.56 ± 0.27 | 0/4 | | | | |
| DM 32 | 0.34 ± 0.02 | 4/18 | | 1/18 | 1/18 | 2/18 |
| BMP 10 | 0.59 ± 0.07 | 2/9 | | | | 2/9 |
| BMP 30 | 0.62 ± 0.09 | 1/9 | | | 1/9 | |
| BMP 100 | 0.70 ± 0.08 | 1/10 | | 1/10 | | |
| E 100 | 0.58 ± 0.06 | 3/10 | 1/10 | 1/10 | | 1/10 |

^{a}p < 0.01 vs the 32 wk groups; Other significant differences demonstrated in figures
Values are means ± SEM

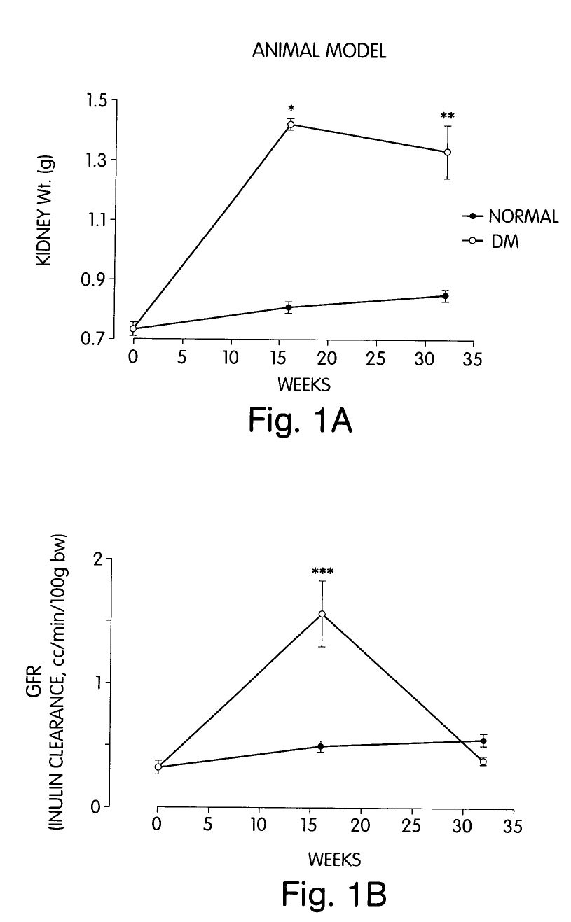

Characterization of the Streptozotocin Diabetic Nephropathy Model: The effects of diabetes on kidney weight and GFR are shown in FIG. 1. DM was induced at week 0. At 16 weeks of DM, kidney weight had increased 1.8 fold compared to normal (1.42±0.02 versus 0.81±0.02 g, p<0.01), while GFR increased 3.2-fold compared to normal (1.56±0.27 versus 0.49±0.04 ml/mim/100 g body wt, p<0.01). These data demonstrated that we had induced significant renal hypertrophy in this animal model. All treatments began at 16 weeks and continued through 32 weeks (or for an additional 16 weeks).

After 16 weeks of vehicle treatment, the kidney weights were not changed (1.44±0.04 g versus 1.42±0.02 g) (FIG. 1). But the GFR of vehicle treated diabetic rats was decreased 75% to significantly lower than normal rats maintained for 32 weeks (0.34□0.02 versus 0.55±0.02 ml/min/100 gbw, p<0.05). This demonstrated that without treatment there was a change from renal hyperfiltration to renal failure in control rats during the treatment period.

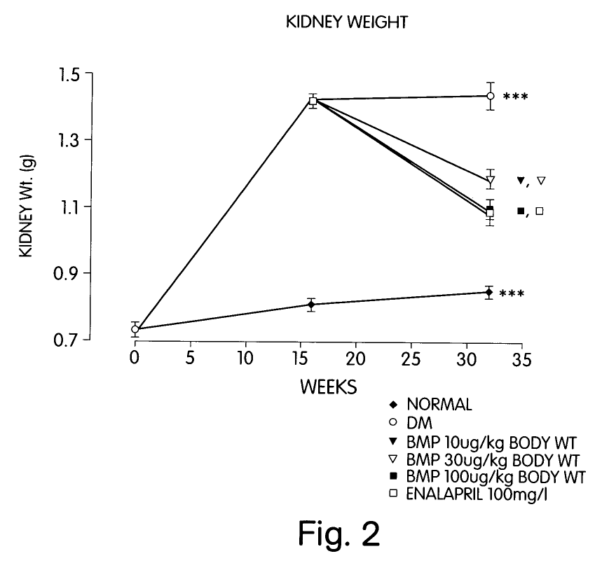

On the other hand, as shown in FIG. 2, BMP-7 and enalapril treated animals had significantly decreased kidney weights (1.10±0.03 in the BMP-7 high dose group, 1.09±0.04 in enalapril treated group) compared to the diabetic vehicle-treated rats (p<0.01). There was a dose-dependent ordering of kidney weights in the BMP-7 treated group (1.19±0.03, 1.19±0.03, 1.10±0.03, respectively). These data demonstrated that BMP-7 and enalapril treatment partially reversed diabetic renal hypertrophy.

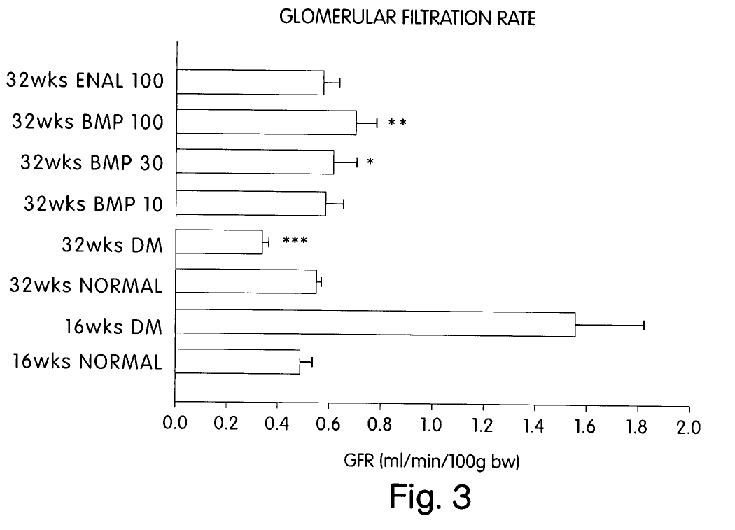

Effects of Therapy on Kidney Hypertrophy: While kidney weight was not affected by 16 weeks of vehicle treatment, GFR was reduced from 1.56±0.27 ml/min/100 g bw to 0.34±0.02, which was 40% reduced from normal of 0.55±0.02 (p<0.05) (FIGS. 1 and 3). The GFR in the BMP-7 and enalapril treatment groups was normal or greater (FIG. 3). The GFR of the BMP-7 high dose group was significantly greater than the GFR of the DM group (0.70±0.08 versus 0.34±0.02, p<0.01). There was a dose-dependent ordering of GFR in the BMP-7 treated groups (0.59±0.07, 0.61±0.09, and 0.70±0.08, respectively). These data indicated that the vehicle control group had developed progressive renal failure during the 16 weeks of vehicle, while the treatment groups had partial reversal of renal hypertrophy and prevention of further injury resulting in GFR significantly greater than normal at the high dose BMP-7 and dose dependently decreasing to normal with decreasing BMP-7 doses.

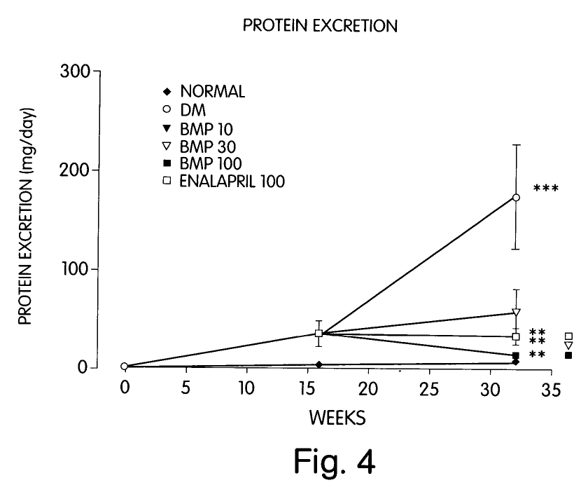

Urine Protein Excretion: The effects of diabetes and treatment on urine protein excretion are shown in FIG. 4. Diabetic rats exhibited a pronounced increase in protein excretion rate compared with nondiabetic rats at both 16 weeks (35.63±13.35 versus 3.76±0.39 mg/day) and 32 weeks (174.44±52.50 versus 8.24±1.28, p<0.01). This response to diabetes was reversed by BMP-7 and enalapril treatment (p<0.01, DM versus BMP10; p<0.001, DM versus BMP30, BMP100 and Enalapril). There was a significant dose-dependent ordering of protein excretion in the BMP-7 treated groups (59.46±21.84, 33.02±9.11, and 14.27±3.50, respectively, p<0.05 comparing low dose to high dose BMP-7).

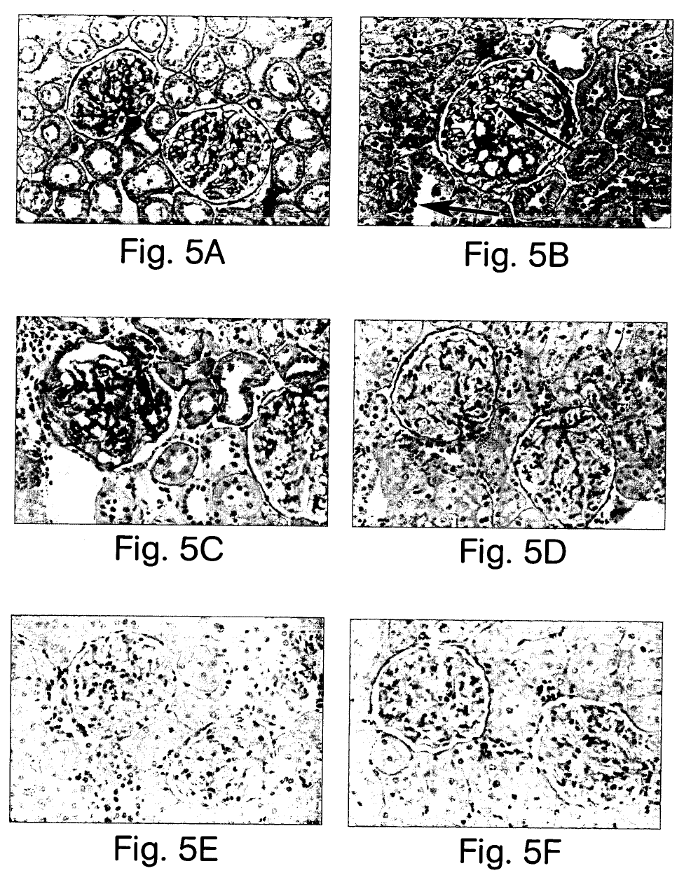

Treatment Effects on Pathology: Compared to normal, kidneys of 16 week diabetic rats had massive glomerular hypertrophy to go along with the increase in kidney weights (FIG. 5). The 16 week diabetic kidneys also had mild increases in mesangial matrix and thickening of glomerular and tubular basement membranes (FIG. 5) in agreement with previous studies (16). By 32 weeks of diabetes, the kidneys of vehicle treated rats continued to be hypertrophied, but a significant component of glomerular area was sclerotic in a focal segmental pattern (FIGS. 5 and 23). The sclerotic areas demonstrated increased matrix, obliteration of capillaries and sparseness of cellular elements. Electron microscopy confirmed the increase in GBM thickness produced by diabetes by 16 weeks as shown in FIG. 5 (data not shown). The focal-segmental nature of glomerular sclerosis in streptozotocin diabetic nephropathy is a known deviation from the human disease pathology of a diffuse global sclerosis with intermittent Kimmelstiel-Wilson nodular glomerular sclerosis lesions (17). This difference has been previously documented (13)(17).

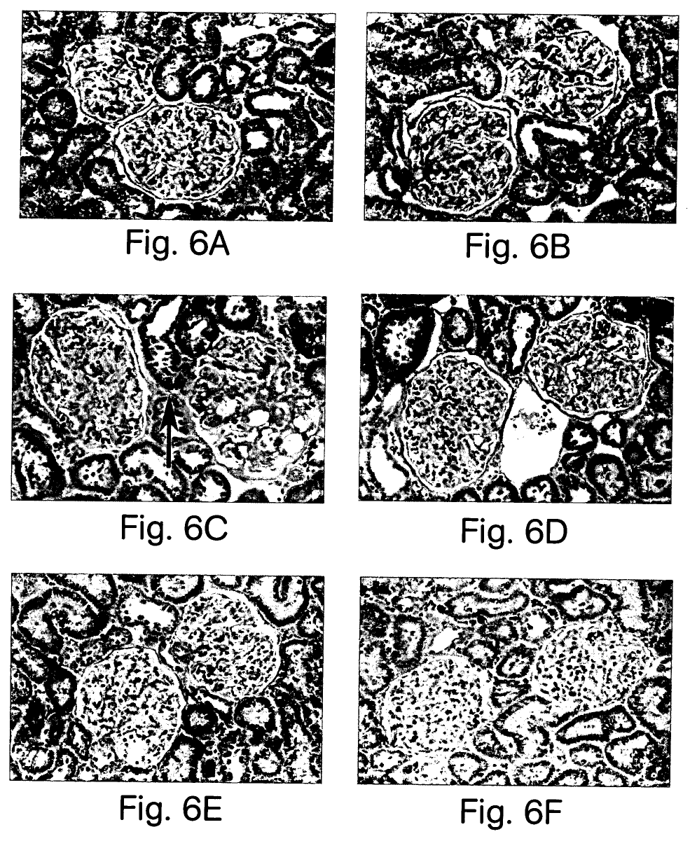

Treatment with BMP-7 prevented the development of glomerulosclerosis along with a dose dependent reduction in the accumulation of mesangial matrix such that the intermediate and high dose BMP-7 treatments were associated with near normal glomerular histology except for incomplete resolution of glomerular hypertrophy (FIGS. 5-7). Enalapril therapy was less effective in preventing glomerulosclerosis, and it was less effective than intermediate or high dose BMP-7 in eliminating accumulation of mesangial matrix (FIG. 5).

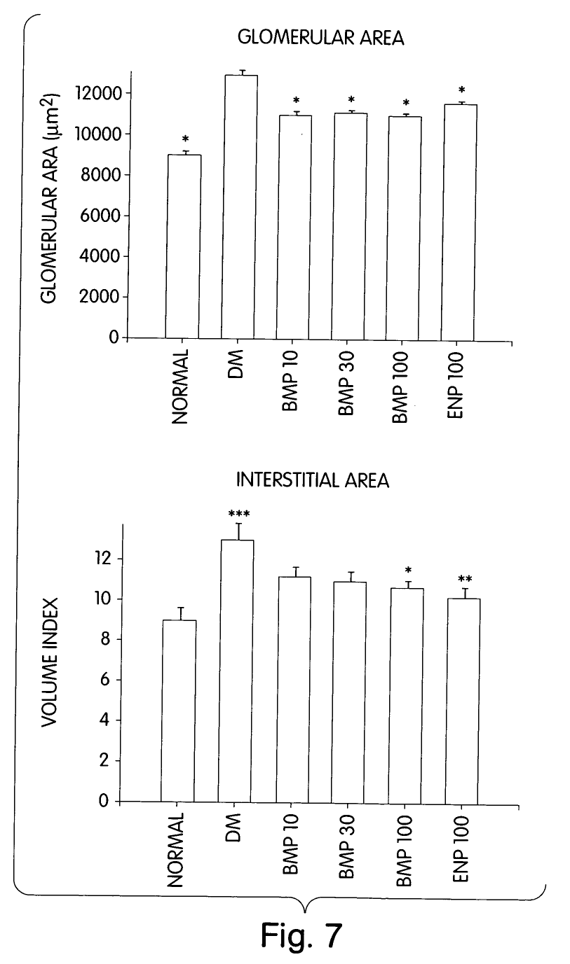

Morphometric Parameters: The effect of diabetes and the various treatments on glomerular area are shown in FIG. 7A. Diabetic rats had a massively larger glomerular area than normal control rats (1.28±0.03 versus 0.90±0.02×104 μm2, p<0.001) concordant with their increased kidney weights. All the treatments except vehicle partially reversed the glomerular hypertrophy (p<0.001). The mesangial matrix area was also increased in diabetic rats compared to all the BMP-7 and enalapril treatment groups, but the ratio of mesangial matrix area to glomerular area were not different between different groups (data not shown).

As shown in FIG. 7B, the cortical interstitial volume was increased from 9.0±0.6% in nondiabetic rats to 13.1±0.7% in the DM rats. High dose BMP-7 and Enalapril treatments significantly reduced the increase in interstitial volume to 10.7±0.3%; and 10.3±0.4%, respectively, p<0.01. DM significantly increased the monocyte/macrophage infiltration both in the glomeruli (2.08±0.24 vs. 1.01±0.17, p<0.01) and in the cortical tubulointerstitium (7.25±0.51 vs. 4.30±0.35, p<0.001) compared to normal rats. BMP-7 and Enalapril treatment decreased the tubulointerstitial infiltration.

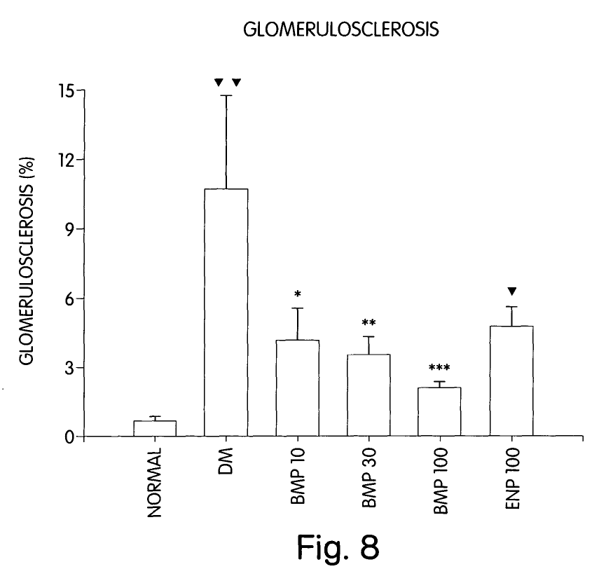

The effect of diabetes, BMP-7, or enalapril treatment on glomerular pathology is shown in FIG. 8. Focal segmental glomerulosclerosis (FSGS) developed in diabetic rats compared with nondiabetic rats at 32 weeks (10.7±4.0% versus 0.7±0.2%, p<0.001). This degree and type of glomerusclerosis is similar to that reported in previous studies with STZ DM (13)(17). Glomerulosclerosis was markedly reduced by BMP-7 and less so by Enalapril treatment. There was a dose-dependent ordering of the reduction in glomerulosclerosis in the BMP-7 treated groups (4.1±1.4%, p<0.05; 3.5±0.8%, p<0.01; and 2.1±0.3%, p<0.001, respectively) (FIG. 8).

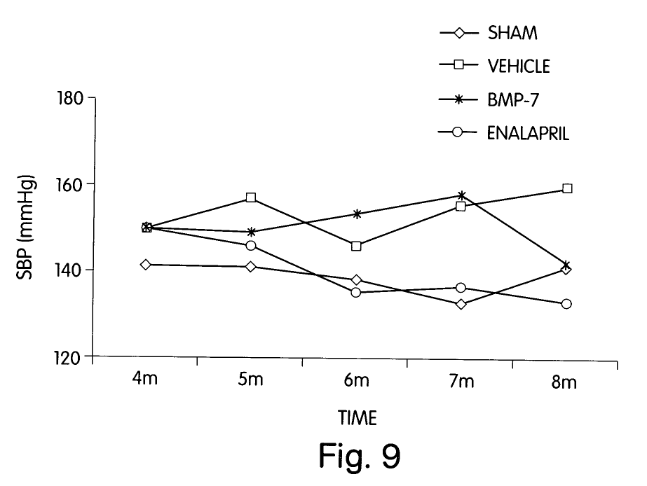

Mechanism of BMP-7 action: A significant component of the renoprotective actions of the positive control in these studies, Enalapril, is attributed to control of hypertension in diabetes (18)(19)(20,21). Therefore, we compared blood pressures between the various treatment groups in this study. As shown in FIG. 9, the 16 week diabetic rats were mildly hypertensive in agreement with previous studies. Mean systolic blood pressures were 160±3 compared to 141±4 in normal control rats (17). By 20 weeks, Enalapril therapy had reversed the hypertension of the diabetic rats, and Enalapril therapy maintained normotension throughout the rest of the 32 weeks. In contrast, BMP-7 therapy, had no effect on blood pressure until after 28 weeks when it began to cause reductions in hypertension until, at 32 weeks, blood pressures were normalized in the BMP-7 high dose treated animals. At 32 weeks the diabetic vehicle treated rats exhibited worsening systolic hypertension (FIG. 9) and widening of their pulse pressures consistent with increasing loss of vascular pliability.

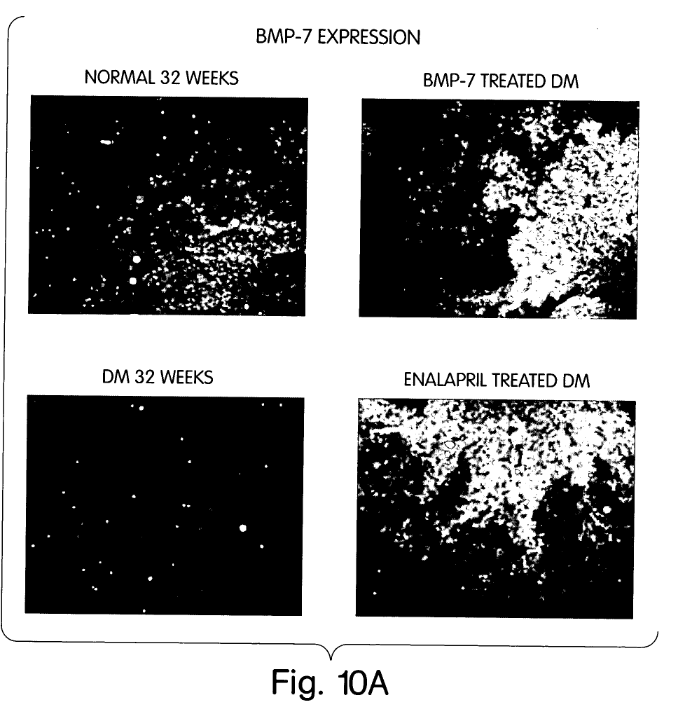

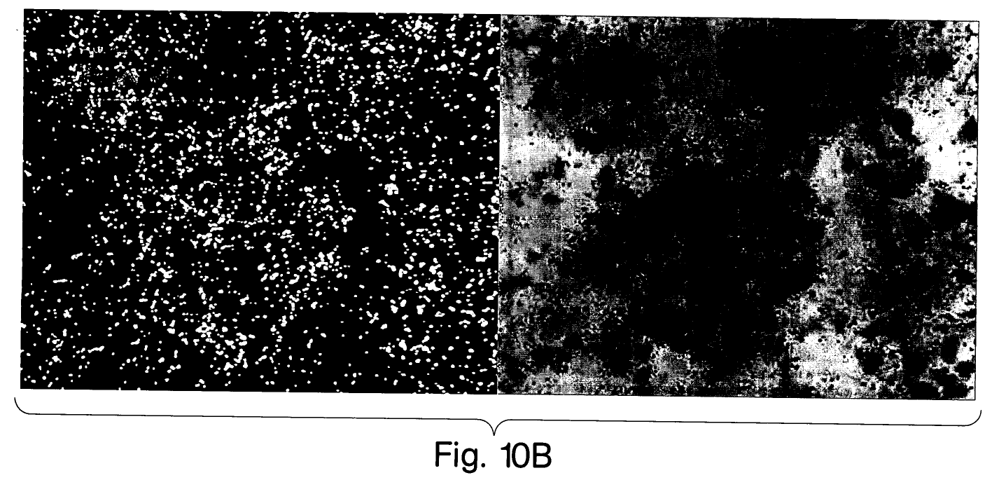

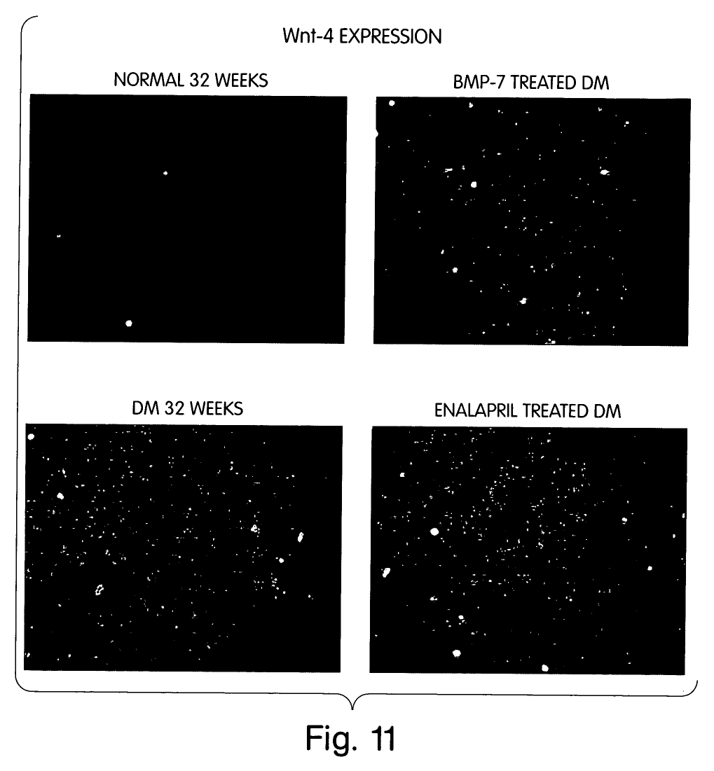

In further pursuit of the mechanism of the renoprotective actions of BMP-7 therapy, we reasoned that a critical pathogenetic action of diabetes might be induced loss of expression of a key differentiation factor/s, such as BMP-7, enabling the ill-fated injury response to be initiated. This turned out to be so as shown in FIG. 10. In the normal rat kidney BMP-7 is expressed in the medulla, the cortical collecting ducts and glomerular podocytes (22). By 16 weeks of diabetes, BMP-7 expression was absent from the kidney, and this absence was maintained during therapy with vehicle (FIG. 10). Treatment with either BMP-7 or Enalapril restored BMP-7 expression in its normal pattern at high levels (FIG. 10). The significance of the changes in BMP-7 expression is related to the expression of another critical kidney developmental morphogen, Wnt4, during renal injury. Wnt4 is normally expressed during kidney development following BMP-7 at the time of epithelializiation of the condensing metanephric mesenchyme around the tip of the ureteric bud (7,8). Wnt4 along with a reciprocal signal from the ureteric bud (BMP-7) is required for epithelial differentiation of the mesenchyme and tubule formation of the condensing mesenchyme leading to formation of the comma and S-shaped bodies and glomerular development. However, in tubulointerstitial injuries, Wnt4 is re-expressed but in the absence of key factors (probably the reciprocal developmental differentiation factor) it promotes interstitial fibrosis (15). As shown in FIG. 11, diabetic injury is associated with Wnt4 expression throughout the kidney, and BMP-7 and Enalapril therapy do not affect Wnt4 expression. During development Wnt4 and BMP-7 interact in the development of the nephron. In the absence of BMP-7 such as in renal injuries, Wnt4 re-expression promotes TGF® induced signaling (23), and this is the likely mechanism by which Wnt4 re-expression appears to promote renal fibrogenesis (15). As shown above, BMP-7 and Enalapril therapy stimulate a successful repair reaction and reinduction of BMP-7 expression. This may indicate that the Wnt4 actions were channeled into the successful repair reaction similar to its role in nephrogenesis.

Discussion

The data presented here demonstrate that the long-term model of streptozotocin induced diabetes is associated with the development of nephropathy and renal failure that resembles the clinical course of human diabetic nephropathy associated with Type I diabetes (24,25). By 16 weeks massive renal hypertrophy, hyperfiltration and proteinuria were established in our model. At that time, the earliest changes of glomerular mesangial matrix accumulation were detectable as previously reported (16). Over the ensuing 16 weeks during the course of the various therapies utilized here, diabetic renal injury either progressed or was partially reversed. In the vehicle treated group, diabetic injury progressed leading to glomerular sclerosis, worsening of the proteinuria, the development of nephrotic syndrome and the development of renal insufficiency. The progression of the nephropathy to severe proteinuria and renal insufficiency was due to progression of the diabetic glomerular pathology to the level of 10% of the glomeruli being sclerotic. This severity of disease is similar to previous reports of glomerulosclerosis in STZ induced DM nephropathy (13,17,26-28).

In three groups of diabetic rats, BMP-7 was administered twice a week intravenously at (10, 30, and 100 μg/kg/bw). The effects of BMP-7 therapy were profound. BMP-7 partially restored kidney weights towards normal, restored glomerular filtration rate to normal, dose dependently eliminated proteinuria, partially reversed glomerular hypertrophy, reversed the increase in the expanded interstitial volume and prevented the development of glomerular sclerosis. The reversal of renal injury was the mechanism by which kidney weights, glomerular area, and glomerular filtration rate were reduced. All of the parameters of nephropathy that were assessed demonstrated dose ordering in their response that was small though present. Only the differences in protein excretion between the low dose and high dose groups achieved statistical significance.

The therapeutic effects of BMP-7 were further characterized by direct comparison to the known therapeutic actions of Enalapril, an angiotensin converting enzyme (ACE) inhibitor. ACE inhibition was first shown to be a renal disease therapeutic agent in STZ diabetes (18,19)(16)(20), and it is especially effective in human diabetic nephropathy (21,29)(30). Subsequently, along with AT-1 receptor blockade, it has become the main renal disease therapeutic for slowing progression of disease (29)(30). In the design of the studies reported here, a treatment of established disease was used. High dose BMP-7 was more effective than Enalapril in reversing proteinuria, maintaining GFR, decreasing mesangial matrix expansion and preventing the development of glomerular sclerosis. BMP-7 and Enalapril therapies were equally effective in reducing kidney weights, reversing glomerular hypertrophy and decreasing interstitial volume expansion. Enalapril therapy normalized blood pressure during the entire 16 week course of therapy. An important component of the therapeutic effectiveness of Enalapril is estimated to be due to the control of systemic and glomerular hemodynamics (18-20). BMP-7 did not affect blood pressure until late in the course of the study, at a time when systolic hypertension was becoming prominent and vascular calcification was present (Davies and Hruska, data not published). We interpret these data to correlate with the actions of BMP-7 to prevent vascular calcification (31). The doses of Enalapril used here (20 mg/kg) were maximal (1,14,32). They were 10-50 fold greater than the doses used in clinical medicine. Preliminary studies demonstrated that there was no difference in the Enalapril response between 5 and 20 mg/kg. Furthermore, we have demonstrated that addition of Enalapril to the drinking water is equally effective as more arduous means of administering Enalapril such as interperitoneal injections and oral/esophageal gavage (Hruska, Wang unpublished data). Therefore, we conclude that BMP-7 therapy of streptozotocin induced diabetic nephropathy in the rat is at least equally effective to Enalapril therapy, and in the area of preventing progression to glomerular sclerosis clearly more effective in this study.

BMP-7 is a critical renal morphogen that is expressed in the normal adult kidney and lost through the influence of renal injury as shown here in diabetic nephropathy. Its therapeutic actions in renal disease appear to be related to recapitulation of its developmental actions. BMP-7 stimulates epithelial differentiation and provides metanephric mesenchymal survival signal during morphogenesis (4)(5)(6). This is similar to the actions of BMP-7 during renal tubulointerstitial injury (1). BMP-7 decreases expression of markers of injury response such as vimentin and α1(I) procollagen, α1(IV) collagen, and it increases expression of epithelial phenotype markers such as E-cadherin (1,5,9,12,22,33) (Hruska, unpublished). It prevents tubular epithelial apoptosis leading to injury prevention and disease resistance (1)(12). BMP-7 is expressed in the Wolffian duct at the time of ureteric bud development (4). It continues to be expressed in the ureteric bud and the developing collecting duct throughout development (34). In addition, BMP-7 is expressed in and required for the condensing metanephric mesenchyme at the tip of the ureteric bud and in the pretubular aggregates beginning at day 12.5 (4). BMP-7 expression subsequently disappears from the comma and S-shaped bodies. In the adult kidney BMP-7 is expressed in glomerular podocytes, the thick ascending limb, distal tubule, and collecting duct (22). The strongest expression is in the collecting duct especially in the medullary segments. In the absence of BMP-7, condensation of metanephric mesenchyme is diminished and mesenchymal cells apoptose between days 12 and 14 dpc (4). This is despite expression of Wnt 4, another critical tubular developmental morphogen expressed in the condensing metanephric mesenchyme at the time of formation of the pretubular aggregates (8). Wnt 4 is required and sufficient for induction of tubulogenesis from day 14 dpc through development of the early nephrons (8). Wnt 4 expression is diminished and absent from the Sshaped bodies following its inductive actions. From their overlapping expression patterns between day 12.5 and 14 dpc, the phenotype of their knockouts, and the overlapping times of their required presence, it is clear that Wnt4 and BMP-7 interact during renal development (4)(5)(7,8). In the adult kidney, Wnt4 expression is limited to the terminal medullary collecting duct (15). However, increased Wnt 4 expression is observed throughout the collecting duct in response to renal injuries while BMP-7 expression is lost, as shown here and elsewhere (1,7,15). Here we show that diabetic nephropathy causes an even more widespread expression of Wnt4 than ureteral obstruction. Wnt4 expression in response to renal injury promotes tubulointerstitial fibrosis (15), and it promotes epithelial to mesenchymal transdifferentiation (EMT) similar to TGFβ (15,35). This is a critical mechanism of producing interstitial myofibroblasts and promoting fibrogenesis (36). These actions of WNT4 are similar to those of TGFβ in renal injury. The mechanism of the relationship between Wnt4 and TGF□ in renal injury is that Wnt 4 signaling stabilizes β-catenin and increases nuclear β-catenin levels (37,38). In the nucleus β-catenin binds in a transcriptional complex with SMAD 4 (23). SMAD 4 in the nucleus exists as a dimer containing a regulatory SMAD. TGF-β induces the regulatory SMAD's 2 and 3, and the SMAD 2/4 and 3/4 dimers in the transcriptional complex associated with β-catenin and the TCF/LEF family regulate gene transcription. The Wnt4/TGFβ interaction leads to promotion of renal fibrosis and EMT (15)(23). Since TGF-β is increased in response to diabetic injury (26,39)(27)(28), and BMP-7 is decreased (data presented here) (40), activation of Wnt4 leads to synergism with TGF-β (23). In the presence of BMP-7, TGF-β signaling is inhibited (9). BMP-7 signaling interacts with Wnt 4 expression leading to the formation of transcriptional complexes containing the BMP-7 stimulated regulatory SMAD's 1,5, and 8 as in development. Thus, the transcriptional signaling induced by TGF-β is competitively inhibited. In addition, BMP-7 stimulates the inhibitory SMAD, SMAD 6 (9), and in the proximal tubule, SMAD 7 (41), which further inhibit TGF-β induced signaling.

The STZ induced DM rat model and type I human DM overexpress TGFβ1,2, and 3 and the TGFβ RII in renal tubular and glomerular cells (26,27,27,28,39,42). TGFβ and the receptor together constitute a major biologic signal inducing a switch toward a profibrotic cellular phenotype (43). Here we demonstrate that another profibrotic cytokine, Wnt4, that synergizes with TGFβ is upregulated in STZ DM. We propose that Wnt4 upregulation may be a common response to renal injury, which, in the absence of BMP-7, stimulates a failed injury response through its interactions with TGFβ.

In summary, the proof of concept studies reported here demonstrate successful treatment of streptozotocin induced diabetic nephropathy in renal insufficiency with BMP-7. BMP-7 effects were equal to and in some respects more potent than a positive control, Enalapril therapy. Diabetic injury induced diffuse Wnt-4 expression representing an injury response and reexpression of a developmental morphogen. In the absence of BMP-7, Wnt4 may synergize with TGF□ in stimulating diabetic injury. BMP-7 therapy restored tubular epithelial phenotype, BMP-7 expression, and inhibited TGFβ actions. As a result a significant positive repair reaction was produced by BMP-7 and glomerular sclerosis was prevented as well as the development of renal insufficiency.

### REFERENCE LIST

1. Hruska, K. A., Guo, G., Wozniak, M., Martin, D., Miller, S., Liapis, H., Loveday, K., Klahr, S., Sampath, T. K., and Morrissey, J. 2000. Osteogenic protein-1 (OP-1) prevents renal fibrogenesis associated with ureteral obstruction. Am J Phys (Renal) 279:F130-F143.
2. Grobstein, C. 1953. Morphogenetic interactions between embyronic mouse tissues separated by a membrane filter. Nature 172:869-871.
3. Saxen, L. 1987. Organogenesis of the Kidney. Camridge University Press, Cambridge, UK.
4. Luo, G., Hofmann, C., Bronckers, A. L., Sohocki, M., Bradley, A., and Karsenty, G. 1995. BMP-7 is an inducer of nephrogenesis, and is also required for eye development and skeletal patterning. Genes Dev. 9:2808-2820.
5. Dudley, A. T., Lyons, K. M., and Robertson, E. J. 1995. A requirement for bone morphogenetic protein-7 during development of the mammalian kidney and eye. Genes Dev 9:2795-2807.
6. Vukicevic, S., Kopp, J. B., Luyten, F. P., and Sampath, T. K. 1996. Induction of nephrogenic mesenchyme by osteogenic protein 1 (bone morphogenetic protein 7). Proc. Natl. Acad. Sci. U.S.A. 93:9021-9026.
7. Stark, K., Vainio, S., Vassileva, G., and McMahon, A. P. 1994. Epithelial transformation of metanephric mesenchyme in the developing kidney regulated by Wnt-4. Nature 372: 679-683.
8. Kispert A., Vainio S., and McMahon, A. P. 1998. Wnt-4 is a mesenchymal signal for epithelial transformation of metanephric mesenchyme in the developing kidney. Development 125:4225-4234.
9. Dorai, H., Vukicevic, S., and Sampath, T. K. 2000. Bone morphogenetic protein-7 (osteogenic protein-1) inhibits smooth muscle cell proliferation and stimulates the expression of markers that are characteristic of SMC phenotype in vitro. J Cellular Physiol 184:37-45.
10. Simon, M., Maresh, J. G., Harris, S. E., Hernandez, J. D., Arar, M., Olson, M. S., and Abboud, H. E. 1999. Expression of bone morphogenetic protein-7 mRNA in normal and ischemic adult rat kidney. Amer. J. Physiol. 276:F382-F389.
11. Wang, S. and Hirschberg, R. 2000. Loss of renal tubular BMP7 during the evolution of experimental diabetic nephropathy. J. Am. Soc. Nephrol. 11:655A (Abstr.)
12. Vukicevic, S., Basic, V., Rogic, D., Basic, N., Shih, M.-S., Shepard, A., Jin, D., Dattatreyamurty, B., Jones, W., Dorai, H. et al. 1998. Osteogenic protein-1 (Bone 30 morphogenetic protein-7) reduces severity of injury after ischemic acute renal failure in rat. J. Clin. Invest. 102:202-214.
13. Lal, M. A., Korner, A., Matsuo, Y., Zelenin, S., Cheng, S. X. J., Jaremko, G., DiBona, G. F., klöf, A.-C., and Aperia, A. 2000. Combined antioxidant and COMT inhibitor treatment reverses renal abnormalities in diabetic rats. Diabetes 49:1381-1389.
14. Ishidoya, S., Morrissey, J., McCracken, R., Reyes, A., and Klahr, S. 1995. Angiotensin II receptor antagonist ameliorates renal tubulointerstitial fibrosis caused by unilateral ureteral ligation. Kidney Int. 47:1285-1294.
15. Surendran, K., McCaul, S. P., and Simon, T. C. 2002. A role for Wnt-4 in renal fibrosis. Am J Physiol Renal Physiol 282:F431-F441.
16. Sassy-Prigent, C., Heudes, D., Jouquey, S., Auberval, D., Belair, M.-F., Michel, O., Hamon, G., Bariety, J., and Bruneval, P. 1995. Morphometric detection of incipient glomerular lesions in diabetic nephropathy in rats. Lab Invest 73:64-71.
17. Remuzzi, A., Perico, N., Amuchastegui, c. s., Malanchini, B., Mazerska, M., Battaglia, C., Bertani, T., and Remuzzi, G. 1993. Short- and long-term effect of angiotensin II receptor blockade in rats with experimental diabetes. J Am Soc Neph 4:40-49.
18. Zatz, R., Dunn, B. R., Meyer, T. W., Anderson, S., Rennke, H. G., and Brenner, B. M. 1986. Prevention of diabetic glomerulopathy by pharmacological amelioration of glomerular capillary hypertension. J. Clin. Invest. 77:1925-1930.
19. Anderson, S., Rennke, H. G., Garcia, D. L., and Brenner, B. M. 1989. Short and long-term effects of antihypertensive therapy in the diabetic rat. Kidney International 36:526-536.
20. Katoh, M., Ohmachi, Y., Kurosawa, Y., Yoneda, H., Tanaka, N., and Narita, H. 2000. Effects of imidapril and captopril on streptozotocin-induced diabetic nephropathy in mice. Eur J Pharmacol 398:381-387.
21. Lewis, E. J., Hunsicker, L. G., Bain, R. P., and Rohde, R. D. 1993. The effect of angiotensin converting-enzyme inhibition on diabetic nephropathy. NEJM 329:1456-1462.
22. Gould, S. E., Day, M., Jones, S., and Dorai, H. 2002. BMP-7 regulates chemokine, cytokine, and hemodynamic gene expression in proximal tubule cells. Kidney International 61:51-60.
23. Nishita, M., Hashimoto, M. K., Ogata, S., Laurent, M. N., Ueno, N., Shibuya, H., and Cho, K. W. Y. 2000. Interaction between Wnt and TGF-β signalling pathways during formation of Spemann's organizer. Nature 403:781-785.
24. Sharma, K., Yulin, J., Guo, J., and Ziyadeh, J. 1996. Neutralization of TGF-B by anti-TGF-β antibody attenuates kidney hypertrophy and the enhanced extracellular matrix gene expression in streptozotocin-diabetic mice. Diabetes 45:522-530. 31
25. Wilson, G. L. and Letter, E. H. 1990. Streptozotocin interactions with pancreatic beta cells and the induction of insulin-dependent diabetes. Curr Top Microbiol Immunol 156:27-54.
26. Yamamoto, T., Nakamura, T., Noble, N. A., Ruoslahti, E., and Border, W. 1993. Expression of transforming growth factor β is elevated in human and experimental diabetic nephropathy. Proc. Natl. Acad. Sci. U.S.A. 90:1814-1818.
27. Nakamura, T., Fukui, M., Ebihara, I., Osada, S., Nagaoka, I., Tomino, Y., and Koide, H. 1993. mRNA expression of growth factors in glomeruli from diabetic rats. Diabetes 42:450-456.
28. Shankland, S. J. and Scholey, J. W. 1994. Expression of transforming growth factor B1 during diabetic renal hypertrophy. Kidney International 46:430-442.
29. Mathiesen, E. R., Hommel, E., Giese, J., and Parving, H. H. 1991. Efficacy of captopril in postponing nephropathy in normotensive insulin dependent diabetic patients with microalbuminuria. British Medical Journal 303:81-87.
30. Wiegmann, T. B., Herron, K. G., Chonko, A. M., MacDougall, M. L., and Moore, W. V. 1992. Effect of angiotensin-converting enzyme inhibition on renal function and albuminuria in normotensive type I diabetic patients. Diabetes 41:62-67.
31. Davies, M. R., Petrosova, T., and Hruska, K. A. 2001. BMP-7 is an effective treatment for vascular calcification in chronic kidney disease (CKD). J. Am. Soc. Nephrol. (Abstr.)
32. Morrissey, J., Hruska, K., Guo, G., Wang, S., Chen, Q., and Klahr, S. 2002. Bone morphogenetic protein-7 improves renal fibrosis and accelerates the return of renal function. J. Am. Soc. Nephrol. 13:S14-S21.
33. Zeisberg, M., Maeshima, Y., Hanai, J.-I., Strutz, F., Dorai, H., and Kalluri, R. 2001. Bone morphogenic protein-7, an inducer of tubulogenesis in kidney development, is an inhibitor of chronic progressive renal disease. J Am Soc Neph 12:830A (Abstr.)
34. Lyons, K. M., Hogan, B. L., and Robertson, E. J. 1995. Colocalization of BMP 7 and BMP 2 RNAs suggests that these factors cooperatively mediate tissue interactions during murine development. Mech. Dev. 50:71-83.
35. Hay, E. D. and Zuk, A. 1995. Transformations between epithelium and mesenchyme: normal, pathological, and experimentally induced. Am J Kidney Dis 26:678-690.
36. Iwano, M., Plieth, D., Danoff, T. M., Xue, C., Okada, H., and Neilson, E. G. 2002. Evidence that fibroblasts derive from epithelium during tissue fibrosis. J. Clin. Invest. 110:341-350.
37. Cox, R. T. and Peifer, M. 1998. Wingless signaling: the inconvenient complexities of life. Current Biology 8:R140-R144.
38. Hartmann, C. and Tabin, C. J. 2000. Dual roles of Wnt signaling during chondrogenesis in the chicken limb. Development 127:3141-3159. 32
39. Hill, C., Flyvbjerg, A., Grøonbæk, H., Petrik, J., Hill, D. J., Thomas, C. R., Sheppard, M. C., and Logan, A. 2000. The renal expression of transforming growth factor-□ isoforms and their receptors in acute and chronic experimental diabetes in rats. Endocrinology 141:1196-1208.
40. Wang, S.-N., Lapage, J., and Hirschberg, R. 2001. Loss of tubular bone morphogeneteic protein-7 in diabetic nephropathy. J. Am. Soc. Nephrol. 12:2392-2399.
41. Hill-Kapturczak, N., Truong, L., Thamilselvan, V., Visner, G. A., Nick, H. S., and Agarwall, A. 2000. Smad7-dependent regulation of heme oxygenase-1 by transforming growth factor-β in human renal epithelial cells. J. Biol. Chem. 275:40904-40909.
42. Sharma, V. K., Bologa, R M., Xu, G.-P., Li, B., Mouradian, J., Wang, J., Serur, D., Rao, V., and Suthanthiran, M. 1996. Intragraft TGF-[beta]1 mRNA:

A correlate of interstitial fibrosis and chronic allograft nephropathy. Kidney International 49:1297-1303.
43. Sharma, K. and Ziyadeh, F. N. 1995. Hyperglycemia and diabetic kidney disease. The case for transforming growth factor-beta as a key mediator. Diabetes 44:1139-1146.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

All cited references (including scientific publications, abstracts, etc.), patents, patent application publications are hereby incorporated herein by reference.

1. A method of treating or preventing chronic renal failure in a mammal, comprising conjointly administering to said mammal an OP/BMP morphogen and an Angiotensin-Converting Enzyme Inhibitor (ACEI).

2. A method of treating or preventing chronic renal failure in a mammal, comprising conjointly administering to said mammal an OP/BMP morphogen and an Angiotensin II Receptor Antagonist (AIIRA).

3. A method of treating or preventing chronic renal failure in a mammal, comprising conjointly administering to said mammal an inducer of endogenous OP/BMP morphogen expression and an Angiotensin-Converting Enzyme Inhibitor (ACEI).

4. A method of treating or preventing chronic renal failure in a mammal, comprising conjointly administering to said mammal an inducer of endogenous OP/BMP morphogen expression and an Angiotensin II Receptor Antagonist (AIIRA).

5. A method of treating or preventing chronic renal failure in a mammal, comprising conjointly administering to said mammal an agonist of an OP/BMP morphogen receptor and an Angiotensin-Converting Enzyme Inhibitor (ACEI).

6. A method of treating or preventing chronic renal failure in a mammal, comprising conjointly administering to said mammal an agonist of an OP/BMP morphogen receptor and an Angiotensin II Receptor Antagonist (AIIRA).

7. A method of treating or preventing chronic renal failure in a mammal, comprising introducing into the kidney of said mammal a therapeutically effective amount of renal mesenchymal progenitor cells pre-treated conjointly with an ACEI and an agent that increases the abundance of an OP/BMP morphogen.

8. A method of treating or preventing chronic renal failure in a mammal, comprising introducing into the kidney of said mammal a therapeutically effective amount of renal mesenchymal progenitor cells pre-treated conjointly with an AIIRA and an agent that increases the abundance of an OP/BMP morphogen.

9. The method of claim 7 or 8, wherein the agent is an OP/BMP morphogen.

10. The method of claim 7 or 8, wherein the agent is an inducer of an OP/BMP morphogen.

11. The method of claim 7 or 8, wherein the agent is an agonist of an OP/BMP morphogen receptor.

12. A method for delaying the need for, or reducing the frequency of, chronic dialysis treatments, comprising conjointly administering to a mammal an OP/BMP morphogen and an ACEI.

13. A method for delaying the need for, or reducing the frequency of, chronic dialysis treatments, comprising conjointly administering to a mammal an OP/BMP morphogen and an AIIRA.

14. A method for delaying the need for, or reducing the frequency of, chronic dialysis treatments, comprising conjointly administering to said mammal an inducer of endogenous OP/BMP morphogen expression and an ACEI.

15. A method for delaying the need for, or reducing the frequency of, chronic dialysis treatments, comprising conjointly administering to said mammal an inducer of endogenous OP/BMP morphogen expression and an AIIRA.

16. A method for delaying the need for, or reducing the frequency of, chronic dialysis treatments, comprising conjointly administering to said mammal an agonist of an OP/BMP morphogen receptor and an ACEI.

17. A method for delaying the need for, or reducing the frequency of, chronic dialysis treatments, comprising conjointly administering to said mammal an agonist of an OP/BMP morphogen receptor and an AIIRA.

18. A method as in any one of claims 1-17, wherein said mammal is afflicted with a condition selected from: chronic renal failure (CRF), end-stage renal disease (ESRD), chronic diabetic nephropathy, diabetic glomerulopathy, diabetic renal hypertrophy, hypertensive nephrosclerosis, hypertensive glomerulosclerosis, chronic glomerulonephritis, hereditary nephritis, or renal dysplasia.

19. A method as in any one of claims 1-17, wherein examination of a renal biopsy of said mammal indicates that said mammal is afflicted with a condition selected from: glomerular hypertrophy, tubular hypertrophy, glomerulosclerosis, or tubulo interstitial sclerosis.

20. A method as in any one of claims 1-17, wherein examination of said mammal indicates renal fibrosis.

21. The method of claim 20, wherein said examination is an ultrasound, NMR or CAT scan of said mammal.

22. A method as in any one of claims 1-17, wherein said mammal possesses a number of functional nephron units which is less than about 40% of a number of functional nephron units present in a mammal having intact healthy kidneys.

23. The method of claim 22, wherein said mammal possesses a number of functional nephron units which is less than about 20% of a number of functional nephron units present in a mammal having intact healthy kidneys.

24. The method of any one of claims 1-17, wherein said mammal is a kidney transplant recipient.

25. The method of any one of claims 1-17, wherein said mammal possesses only one kidney.

26. The method of any one of claims 1-17, wherein examination of a urinary sediment of said mammal indicates a presence of broad casts.

27. The method of any one of claims 1-17, wherein said mammal has a GFR which is chronically less than about 40% of a GFRₑₓₚ for said mammal.

28. The method of claim 27, wherein said mammal has a GFR which is chronically less than about 20% of a GFRₑₓₚ for said mammal.

29. The method of any one of claims 1-17, wherein said mammal is a human male weighing at least about 50 kg and has a GFR which is chronically less than about 40 ml/min.

30. The method of any one of claims 1-17, wherein said mammal is a human female weighing at least about 40 kg and has a GFR which is chronically less than about 30 ml/min.

31. The method of any one of claims 1-17, wherein said treatment or prevention reduces serum creatinine levels in said mammal by at least about 5% over 3 months.

32. The method of any one of claims 1-17, wherein prior to said treatment or prevention, said mammal presented a chronic decline in a clinical indicator of renal function, and after at least about 3 months of said treatment or prevention, said indicator stabilizes.

33. The method of any one of claims 1-6 and 12-17, wherein at least one of said ACEI, said AIIRA or said morphogen is administered orally, parenterally, intravenously, intraperitoneally, or into a renal capsule, or by an implanted device.

34. The method of claim 33, wherein a stent has been implanted into said mammal for said administration of at least one of said ACEI, said AIIRA or said morphogen.

35. The method of any one of claims 1-6 and 12-17, wherein at least one of said ACEI or said AIIRA, and at least one of said morphogen are conjointly administered at least once a week for a period of at least about one month.

36. The method of any one of claims 1-6 and 12-17, wherein at least one of said ACEI or AIIRA, and at least one of said morphogen are conjointly administered at least once a week for a period of at least about one year.

37. The method of any one of claims 1-6 and 12-17, wherein said ACEI or said AIIRA, and said morphogen are administered through different routes.

38. The method of any one of claims 1-6 and 12-17, wherein said ACEI or said AIIRA, and said morphogen are conjointly administered at different frequencies.

39. The method of any one of claims 1-6 and 12-17, wherein said morphogen is administered at a dosage of about 0.01-1000 μg/kg body weight of said mammal.

40. The method of claim 39, wherein said morphogen is administered at a dosage of about 10-300 μg/kg body weight of said mammal.

41. The method of any one of claims 1, 3, 5, 12, 14 and 16, wherein said ACEI is administered orally at a concentration of about 1-10,000 mg/L, preferably 10-1000 mg/L, 10-100 mg/L, 100-1000 mg/L, most preferably 100 mg/L.

42. The method of any one of claims 2, 4, 6, 13, 15 and 17, wherein said AIIRA is administered orally at a concentration of about 0.01-100 mg/kg body weight, preferably 0.1-10 mg/kg body weight, 0.2-5 mg/kg body weight, 0.5-2 mg/kg body weight, most preferably 1 mg/kg body weight.

43. The method of any one of claims 1-6 and 12-17, wherein said OP/BMP morphogen and, ACEI or AIIRA are administered in a single pharmaceutical composition.

44. The method of any one of claims 1-6 and 12-17, wherein said OP/BMP morphogen and, ACEI or AIIRA are administered in separate pharmaceutical compositions at or around the same time.

45. The method of any one of claims 1-6 and 12-17, wherein said OP/BMP morphogen and, ACEI or AIIRA are administered in separate pharmaceutical compositions at different times.

46. The method of any one of claims 1-17, wherein said morphogen (a) induces chondrogenesis in an ectopic bone assay; (b) prevents, inhibits, delays or alleviates loss of renal function in an animal model of chronic renal failure, or (c) causes a clinically significant improvement in a standard marker of renal function when administered to a mammal in, or at risk of, chronic renal failure.

47. The method of of any one of claims 1-17, wherein said morphogen comprises a polypeptide including at least a C-terminal cysteine domain of a protein selected from: a pro form, a mature form, or a soluble form of a polypeptide, wherein said polypeptide is: OP-1, OP-2, OP-3, BMP2, BMP3, BMP4, BMP5, BMP6, or BMP9.

48. The method of claim 47, wherein said morphogen comprises a polypeptide including at least a C-terminal cysteine domain of a polypeptide selected from: a pro form, a mature form, or a soluble form of human OP-1.

49. The method of claim 1, wherein said morphogen comprises a polypeptide having at least 70% homology or 50% identity with an amino acid sequence of a C-terminal seven-cysteine domain of human OP-1 (SEQ ID NO: 2).

50. The method of claim 49, wherein said polypeptide has at least 75% homology or 60% identity with an amino acid sequence of a C-terminal. seven-cysteine domain of human OP-1 (SEQ ID NO: 2).

51. The method of claim 49, wherein said polypeptide has at least 80% homology or 70% identity with an amino acid sequence of a C-terminal seven-cysteine domain of human OP-1 (SEQ ID NO: 2).

52. The method of claim 53, wherein said polypeptide has at least 90% identity with an amino acid sequence of a C-terminal seven-cysteine domain of human OP-1 (SEQ ID NO: 2).

53. The method of any one of claims 1, 3, 5, 7, 9-12, 14, and 16, wherein said ACEI is: any one compound of the formulas I-XXVIII or their salts thereof; acylmercapto and mercaptoalkanoyl prolines; captopril (1-[(2S)-3-mercapto-2-methylpropionyl]-L-proline); ether or thioether mercaptoacyl prolines; zofenopril; carboxyalkyl dipeptides; enalapril (N-(1-ethoxycarbonyl-3-phenylpropyl)-L-ananyl-L-proline); lisinopril; quinapril; ramipril; carboxyalkyl dipeptide mimics; cilazapril; benazapril; phosphinylalkanoyl prolines; fosinopril; trandolopril; phosphonamidate substituted amino or imino acids; phosphonate substituted amino or imino acids and salts thereof; ceronapril ((S)-1-[6-amino-2-[[hydroxy](4-phenylbutyl)phosphinyl]oxy]-1-oxohexyl]-L-proline); BRL 36,378; MC-838; CGS 14824 (3-([1-ethoxycarbonyl-3-phenyl-(1S)-propyl]-amino)-2,3,4,5-tetrahydro-2-oxo-1-(3S)-benzazepine-1 acetic acid HCL); CGS 16,617 (3(S)-[[(1S)-5-amino-1-carboxypentyl]amino]2,3,4,5-tetrahydro-2-oxo-1H-1-benzazepine-1-ethanoic acid); Cetapril (alacepril, Dainippon); Ru 44570; Cilazapril; Ro 31-2201; Lisinopril; Indalapril (delapril); Rentiapril (fentiapril, Santen); Indolapril; Spirapril; Perindopril; Quinapril; CI 925 ([3S-[2[R(*)R(*)]]3R(*)]-2-[2-[[1-(ethoxy-carbonyl)-3-phenylpropyl]amino[-1-oxopropyl]-1,2,3,4-tetrahydro-6,7-dimethoxy-3-isoquinolinecarboxylic acid HCL); WY-44221; mercapto-containing compounds; pivopril; YS980; Omapatrilat; Alacepril; moveltopril; quinaprilat; moexipril; perinodpril (S-9490); pentopril; ancovenin; phenacein; or nicotianamin.

54. The method of any one of claims 2, 4, 6, 8-11, 13, 15, and 17, wherein said AIIRA is: Losartan (Cozaar®), Valsartan (Diovan®), Irbesartan (Avapro®), Candesartan (Atacand®), Telmisartan (Micardis®), tasosartan, zolarsartan, Teveten (eprosartan mesylate) or olmesartan medoxomil (Benicar).

55. The method of any one of claims 1, 3, 5, 7, 9-12, 14, and 16, wherein said ACEI is Enalapril.

56. A pharmaceutical composition comprising a therapeutically effective amount an ACE inhibitor and an OP/BMP morphogen formulated with pharmaceutically acceptable salt, carrier, excipient or diluent.

57. A pharmaceutical composition comprising a therapeutically effective amount an AIIRA and an OP/BMP morphogen formulated with pharmaceutically acceptable salt, carrier, excipient or diluent.

58. The pharmaceutical composition of claim 56, wherein the ACE inhibitor is Enalapril.

59. The pharmaceutical composition of claim 57, wherein the AIIRA is: Losartan (Cozaar®), Valsartan (Diovan®), Irbesartan (Avapro®), Candesartan (Atacand®), Telmisartan (Micardis®), tasosartan, zolarsartan, Teveten (eprosartan mesylate) or olmesartan medoxomil (Benicar).

60. The pharmaceutical composition of claim 56 or 57, wherein the morphogen is the polypeptide of SEQ ID NO: 3.

61. The pharmaceutical composition of claim 56 or 57, wherein the morphogen is a first polypeptide including at least a C-terminal cysteine domain of a protein selected from: a pro form, a mature form, or a soluble form of a second polypeptide, wherein said second polypeptide is: OP-1, OP-2, OP-3, BMP2, BMP3, BMP4, BMP5, BMP6, or BMP9.

62. The pharmaceutical composition of claim 56 or 57, wherein said morphogen comprises a polypeptide having at least 70% homology or 50% identity with an amino acid sequence of a C-terminal seven-cysteine domain of human OP-1 (SEQ ID NO: 2).

63. The pharmaceutical composition of claim 62, wherein said polypeptide has at least 75% homology or 60% identity with an amino acid sequence of a C-terminal seven-cysteine domain of human OP-1 (SEQ ID NO: 2).

64. The pharmaceutical composition of claim 62, wherein said polypeptide has at least 80% homology or 70% identity with an amino acid sequence of a C-terminal seven-cysteine domain of human OP-1 (SEQ ID NO: 2).

65. The pharmaceutical composition of claim 62, wherein said polypeptide has at least 90% identity with an amino acid sequence of a C-terminal seven-cysteine domain of human OP-1 (SEQ ID NO: 2).

66. The pharmaceutical composition of claim 56, wherein said ACEI is: any one compound of the formulas I-XXVIII or their salts thereof; acylmercapto and mercaptoalkanoyl prolines; captopril (1-[(2S)-3-mercapto-2-methylpropionyl]-L-proline); ether or thioether mercaptoacyl prolines; zofenopril; carboxyalkyl dipeptides; enalapril (N-(1-ethoxycarbonyl-3-phenylpropyl)-L-ananyl-L-proline); lisinopril; quinapril; ramipril; carboxyalkyl dipeptide mimics; cilazapril; benazapril; phosphinylalkanoyl prolines; fosinopril; trandolopril; phosphonamidate substituted amino or imino acids; phosphonate substituted amino or imino acids and salts thereof; ceronapril ((S)-1-[6-amino-2-[[hydroxy](4-phenylbutyl)phosphinyl]oxy]-1-oxohexyl]-L-proline); BRL 36,378; MC-838; CGS 14824 (3-([1-ethoxycarbonyl-3-phenyl-(1S)-propyl]-amino)-2,3,4,5-tetrahydro-2-oxo-1-(3S)-benzazepine-I acetic acid HCL); CGS 16,617 (3(S)-[[(1S)-5-amino-1-carboxypentyl]amino]2,3,4,5-tetrahydro-2-oxo-1H-1-benzazepine-1-ethanoic acid); Cetapril (alacepril, Dainippon); Ru 44570; Cilazapril; Ro 31-2201; Lisinopril; Indalapril (delapril); Rentiapril (fentiapril, Santen); Indolapril; Spirapril; Perindopril; Quinapril; CI 925 ([3S-[2[R(*)R(*)]]3R(*)]-2-[2-[[1-(ethoxy-carbonyl)-3-phenylpropyl]amino[-1-oxopropyl]-1,2,3,4-tetrahydro-6,7-dimethoxy-3-isoquinolinecarboxylic acid HCL); WY-44221; mercapto-containing compounds; pivopril; YS980; Omapatrilat; Alacepril; moveltopril; quinaprilat; moexipril; perinodpril (S-9490); pentopril; ancovenin; phenacein; or nicotianamin.

67. The pharmaceutical composition of claim 57, wherein said AIIRA is: Losartan (Cozaar®), Valsartan (Diovan®), Irbesartan (Avapro®), Candesartan (Atacand®), Telmisartan (Micardis®), tasosartan, zolarsartan, Teveten (eprosartan mesylate) or olmesartan medoxomil (Benicar).

68. A package pharmaceutical comprising the pharmaceutical composition of any one of claims 56-67, in association with instructions for administering the composition to a mammal for treatment or prevention of chronic renal failure.


SEQUENCE LISTING (31 sequences)

SEQ ID NO: 1 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 97 aa, Homo sapiens
HRRLRSQERREMQREILSILGLPHRPRPHLQGKHNSAPMFMLDLYNAMAVEEGGGPGGQGFSYPYKAVFSTQGPPLASLQDSHFLTDADMVMSFVNL

SEQ ID NO: 3 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 4 - protein, 139 aa, Mus musculus
STGGKQRSQNRSKTPKNQEALRMASVAENSSSDQRQACKKHELYVSFRDLGWQDWIIAPEGYAAYYCEGECAFPLNSYMNATNHAIVQTLVHFINPDTVPKPCCAPTQLNAISVLYFDDSSNVILKKYRNMVVRACGCH

SEQ ID NO: 5 - protein, 139 aa, Homo sapiens
AVRPLRRRQPKKSNELPQANRLPGIFDDVHGSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 6 - protein, 139 aa, Mus musculus
AARPLKRRQPKKTNELPHPNKLPGIFDDGHGSRGREVCRRHELYVSFRDLGWLDWVIAPQGYSAYYCEGECAFPLDSCMNATNHAILQSLVHLMKPDVVPKACCAPTKLSATSVLYYDSSNNVILRKHRNMVVKACGCH

SEQ ID NO: 7 - protein, 588 aa, Drosophila melanogaster
MRAWLLLLAVLATFQTIVRVASTEDISQRFIAAIAPVAAHIPLASASGSGSGRSGSRSGGASTSTALAKAFNPFSEPASFSDSDKSHRSKTNKKPSKSDANRQFNEVHKPRTDQLENSKNMSKQLVNKPNHNKMAVKEQRSHHKKSHHHRSHQPKQASASTESHQSSSIESIFVEEPTLVLDREVASINVPANAKAIIAEQGPSTYSKEALIKDKLKPDPSTLVEIEKSLLSLFNMKRPPKIDRSKIIIPEPMKKLYAEIMGHELDSVNIPKPGLLTKSANTVRSFTHKDSKIDDRFPHHHRFRLHFDVKSIPADEKLKAAELQLTRDALSQQVVASRSSANRTRYQVLVYDITRVGVRGQREPSYLLLDTKTVRLNSTDTVSLDVQPAVDRWLASPQRNYGLLVEVRTVRSLKPAPHHHVRLRRSADEAHERWQHKQPLLFTYTDDGRHKARSIRDVSGGEGGGKGGRNKRQPRRPTRRKNHDDTCRRHSLYVDFSDVGWDDWIVAPLGYDAYYCHGKCPFPLADHFNSTNHAVVQTLVNNMNPGKVPKACCVPTQLDSVAMLYLNDQSTVVLKNYQEMTVVGCGCR

SEQ ID NO: 8 - protein, 360 aa, Xenopus laevis
MVWLRLWAFLHILAIVTLDPELKRREELFLRSLGFSSKPNPVSPPPVPSILWRIFNQRMGSSIQKKKPDLCFVEEFNVPGSVIRVFPDQGRFIIPYSDDIHPTQCLEKRLFFNISAIEKEERVTMGSGIEVQPEHLLRKGIDLRLYRTLQITLKGMGRSKTSRKLLVAQTFRLLHKSLFFNLTEICQSWQDPLKNLGLVLEIFPKKESSWMSTANDECKDIQTFLYTSLLTVTLNPLRCKRPRRKRSYSKLPFTASNICKKRHLYVEFKDVGWQNWVIAPQGYMANYCYGECPYPLTEILNGSNHAILQTLVHSIEPEDIPLPCCVPTKMSPISMLFYDNNDNVVLRHYENMAVDECGCR

SEQ ID NO: 9 - protein, 438 aa, Mus musculus
MRKMQKEILSVLGPPHRPRPLHGLQQPQPPVLPPQQQQQQQQQQTAREEPPPGRLKSAPLFMLDLYNALSNDDEEDGASEGVGQEPGSHGGASSSQLRQPSPGAAHSLNRKSLLAPGPGGGASPLTSAQDSAFLNDADMVMSFVNLVEYDKEFSPHQRHHKEFKFNLSQIPEGEAVTAAEFRVYKDCVVGSFKNQTFLISIYQVLQEHQHRDSDLFLLDTRVVWASEEGWLEFDITATSNLWVVTPQHNMGLQLSVVTRDGLHVNPRAAGLVGRDGPYDKQPFMVAFFKVSEVHVRTTRSASSRRRQQSRNRSTQSQDVSRGSGSSDYNGSELKTACKKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 10 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 11 - DNA, 1751 bp, Homo sapiens
ggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttattt

SEQ ID NO: 12 - protein, 472 aa, Homo sapiens
MAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQFLRKAKENEEFLIGFNITSKGRQLPKRRLPFPEPYILVYANDAAISEPESVVSSLQGHRNFPTGTVPKWDSHIRAALSIERRKKRSTGVLLPLQNNELPGAEYQYKKDEVWEERKPYKTLQAQAPEKSKNKKKQRKGPHRKSQTLQFDEQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 13 - protein, 372 aa, Homo sapiens
MPPPQQGPCGHHLLLLLALLLPSLPLTRAPVPPGPAAALLQALGLRDEPQGAPRLRPVPPVMWRLFRRRDPQETRSGSRRTSPGVTLQPCHVEELGVAGNIVRHIPDRGAPTRASEPVSAAGHCPEWTVVFDLSAVEPAERPSRARLELRFAAAAAAAPEGGWELSVAQAGQGAGADPGPVLLRQLVPALGPPVRAELLGAAWARNASWPRSLRLALALRPRAPAACARLAEASLLLVTLDPRLCHPLARPRRDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 14 - protein, 455 aa, Drosophila melanogaster
MSGLRNTSEAVAVLASLGLGMVLLMFVATTPPAVEATQSGIYIDNGKDQTIMHRVLSEDDKLDVSYEILEFLGIAERPTHLSSHQLSLRKSAPKFLLDVYHRITAEEGLSDQDEDDDYERGHRSRRSADLEEDEGEQQKNFITDLDKRAIDESDIIMTFLNKRHHNVDELRHEHGRRLWFDVSNVPNDNYLVMAELRIYQNANEGKWLTANREFTITVYAIGTGTLGQHTMEPLSSVNTTGDYVGWLELNVTEGLHEWLVKSKDNHGIYIGAHAVNRPDREVKLDDIGLIHRKVDDEFQPFMIGFFRGPELIKATAHSSHHRSKRSASHPRKRKKSVSPNNVPLLEPMESTRSCQMQTLYIDFKDLGWHDWIIAPEGYGAFYCSGECNFPLNAHMNATNHAIVQTLVHLLEPKKVPKPCCAPTRLGALPVLYHLNDENVNLKKYRNMIVKSCGCH

SEQ ID NO: 15 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 16 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 17 - DNA, 1822 bp, Homo sapiens
ggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 18 - DNA, 1873 bp, Mus musculus
ctgcagcaagtgacctcgggtcgtggaccgctgccctgccccctccgctgccacctggggcggcgcgggcccggtgccccggatcgcgcgtagagccggcgcgatgcacgtgcgctcgctgcgcgctgcggcgccacacagcttcgtggcgctctgggcgcctctgttcttgctgcgctccgccctggccgatttcagcctggacaacgaggtgcactccagcttcatccaccggcgcctccgcagccaggagcggcgggagatgcagcgggagatcctgtccatcttagggttgccccatcgcccgcgcccgcacctccagggaaagcataattcggcgcccatgttcatgttggacctgtacaacgccatggcggtggaggagagcgggccggacggacagggcttctcctacccctacaaggccgtcttcagtacccagggcccccctttagccagcctgcaggacagccatttcctcactgacgccgacatggtcatgagcttcgtcaacctagtggaacatgacaaagaattcttccaccctcgataccaccatcgggagttccggtttgatctttccaagatccccgagggcgaacgggtgaccgcagccgaattcaggatctataaggactacatccgggagcgatttgacaacgagaccttccagatcacagtctatcaggtgctccaggagcactcaggcagggagtcggacctcttcttgctggacagccgcaccatctgggcttctgaggagggctggttggtgtttgatatcacagccaccagcaaccactgggtggtcaaccctcggcacaacctgggcttacagctctctgtggagaccctggatgggcagagcatcaaccccaagttggcaggcctgattggacggcatggaccccagaacaagcaacccttcatggtggccttcttcaaggccacggaagtccatctccgtagtatccggtccacggggggcaagcagcgcagccagaatcgctccaagacgccaaagaaccaagaggccctgaggatggccagtgtggcagaaaacagcagcagtgaccagaggcaggcctgcaagaaacatgagctgtacgtcagcttccgagaccttggctggcaggactggatcattgcacctgaaggctatgctgcctactactgtgagggagagtgcgccttccctctgaactcctacatgaacgccaccaaccacgccatcgtccagacactggttcacttcatcaacccagacacagtacccaagccctgctgtgcgcccacccagctcaacgccatctctgtcctctacttcgacgacagctctaatgtcatcctgaagaagtacagaaacatggtggtccgggcctgtggctgccactagctcttcctgagaccctgacctttgcggggccacacctttccaaatcttcgatgtctcaccatctaagtctctcactgcccaccttggcgaggagaacagaccaacctctcctgagccttccctcacctcccaaccggaagcatgtaagggttccagaaacctgagcgtgcagcagctgatgagcgccctttccttctggcacgtgacggacaagatcctaccagctaccacagcaaacgcctaagagcaggaaaaatgtctgccaggaaagtgtccagtgtccacatggcccctggcgctctgagtctttgaggagtaatcgcaagcctcgttcagctgcagcagaaggaagggcttagccagggtgggcgctggcgtctgtgttgaagggaaaccaagcagaagccactgtaatgatatgtcacaataaaacccatgaatgaaaaaaaaaaaaaaaaaaaaaaaaaaaaagaattc

SEQ ID NO: 19 - protein, 430 aa, Mus musculus
MHVRSLRAAAPHSFVALWAPLFLLRSALADFSLDNEVHSSFIHRRLRSQERREMQREILSILGLPHRPRPHLQGKHNSAPMFMLDLYNAMAVEESGPDGQGFSYPYKAVFSTQGPPLASLQDSHFLTDADMVMSFVNLVEHDKEFFHPRYHHREFRFDLSKIPEGERVTAAEFRIYKDYIRERFDNETFQITVYQVLQEHSGRESDLFLLDSRTIWASEEGWLVFDITATSNHWVVNPRHNLGLQLSVETLDGQSINPKLAGLIGRHGPQNKQPFMVAFFKATEVHLRSIRSTGGKQRSQNRSKTPKNQEALRMASVAENSSSDQRQACKKHELYVSFRDLGWQDWIIAPEGYAAYYCEGECAFPLNSYMNATNHAIVQTLVHFINPDTVPKPCCAPTQLNAISVLYFDDSSNVILKKYRNMVVRACGCH

SEQ ID NO: 20 - DNA, 1723 bp, Homo sapiens
ggcgccggcagagcaggagtggctggaggagctgtggttggagcaggaggtggcacggcagggctggagggctccctatgagtggcggagacggcccaggaggcgctggagcaacagctcccacaccgcaccaagcggtggctgcaggagctcgcccatcgcccctgcgctgctcggaccgcggccacagccggactggcgggtacggcggcgacagaggcattggccgagagtcccagtccgcagagtagccccggcctcgaggcggtggcgtcccggtcctctccgtccaggagccaggacaggtgtcgcgcggcggggctccagggaccgcgcctgaggccggctgcccgcccgtcccgccccgccccgccgcccgccgcccgccgagcccagcctccttgccgtcggggcgtccccaggccctgggtcggccgcggagccgatgcgcgcccgctgagcgccccagctgagcgcccccggcctgccatgaccgcgctccccggcccgctctggctcctgggcctggcgctatgcgcgctgggcgggggcggccccggcctgcgacccccgcccggctgtccccagcgacgtctgggcgcgcgcgagcgccgggacgtgcagcgcgagatcctggcggtgctcgggctgcctgggcggccccggccccgcgcgccacccgccgcctcccggctgcccgcgtccgcgccgctcttcatgctggacctgtaccacgccatggccggcgacgacgacgaggacggcgcgcccgcggagcggcgcctgggccgcgccgacctggtcatgagcttcgttaacatggtggagcgagaccgtgccctgggccaccaggagccccattggaaggagttccgctttgacctgacccagatcccggctggggaggcggtcacagctgcggagttccggatttacaaggtgcccagcatccacctgctcaacaggaccctccacgtcagcatgttccaggtggtccaggagcagtccaacagggagtctgacttgttctttttggatcttcagacgctccgagctggagacgagggctggctggtgctggatgtcacagcagccagtgactgctggttgctgaagcgtcacaaggacctgggactccgcctctatgtggagactgaggacgggcacagcgtggatcctggcctggccggcctgctgggtcaacgggccccacgctcccaacagcctttcgtggtcactttcttcagggccagtccgagtcccatccgcacccctcgggcagtgaggccactgaggaggaggcagccgaagaaaagcaacgagctgccgcaggccaaccgactcccagggatctttgatgacgtccacggctcccacggccggcaggtctgccgtcggcacgagctctacgtcagcttccaggacctcggctggctggactgggtcatcgctccccaaggctactcggcctattactgtgagggggagtgctccttcccactggactcctgcatgaatgccaccaaccacgccatcctgcagtccctggtgcacctgatgaagccaaacgcagtccccaaggcgtgctgtgcacccaccaagctgagcgccacctctgtgctctactatgacagcagcaacaacgtcatcctgcgcaaacaccgcaacatggtggtcaaggcctgcggctgccactgagtcagcccgcccagccctactgcag

SEQ ID NO: 21 - protein, 402 aa, Homo sapiens [sequence omitted]

SEQ ID NO: 22 - DNA, 1926 bp, Mus musculus
gccaggcacaggtgcgccgtctggtcctccccgtctggcgtcagccgagcccgaccagctaccagtggatgcgcgccggctgaaagtccgagatggctatgcgtcccgggccactctggctattgggccttgctctgtgcgcgctgggaggcggccacggtccgcgtcccccgcacacctgtccccagcgtcgcctgggagcgcgcgagcgccgcgacatgcagcgtgaaatcctggcggtgctcgggctaccgggacggccccgaccccgtgcacaacccgccgctgcccggcagccagcgtccgcgcccctcttcatgttggacctataccacgccatgaccgatgacgacgacggcgggccaccacaggctcacttaggccgtgccgacctggtcatgagcttcgtcaacatggtggaacgcgaccgtaccctgggctaccaggagccacactggaaggaattccactttgacctaacccagatccctgctggggaggctgtcacagctgctgagttccggatctacaaagaacccagcacccacccgctcaacacaaccctccacatcagcatgttcgaagtggtccaagagcactccaacagggagtctgacttgttctttttggatcttcagacgctccgatctggggacgagggctggctggtgctggacatcacagcagccagtgaccgatggctgctgaaccatcacaaggacctgggactccgcctctatgtggaaaccgcggatgggcacagcatggatcctggcctggctggtctgcttggacgacaagcaccacgctccagacagcctttcatggtaaccttcttcagggccagccagagtcctgtgcgggcccctcgggcagcgagaccactgaagaggaggcagccaaagaaaacgaacgagcttccgcaccccaacaaactcccagggatctttgatgatggccacggttcccgcggcagagaggtttgccgcaggcatgagctctacgtcagcttccgtgaccttggctggctggactgggtcatcgccccccagggctactctgcctattactgtgagggggagtgtgctttcccactggactcctgtatgaacgccaccaaccatgccatcttgcagtctctggtgcacctgatgaagccagatgttgtccccaaggcatgctgtgcacccaccaaactgagtgccacctctgtgctgtactatgacagcagcaacaatgtcatcctgcgtaaacaccgtaacatggtggtcaaggcctgtggctgccactgaggccccgcccagcatcctgcttctactaccttaccatctggccgggcccctctccagaggcagaaacccttctatgttatcatagctcagacaggggcaatgggaggcccttcacttcccctggccacttcctgctaaaattctggtctttcccagttcctctgtccttcatggggtttcggggctatcaccccgccctctccatcctcctaccccaagcatagactgaatgcacacagcatcccagagctatgctaactgagaggtctggggtcagcactgaaggcccacatgaggaagactgatccttggccatcctcagcccacaatggcaaattctggatggtctaagaaggccctggaattctaaactagatgatctgggctctctgcaccattcattgtggcagttgggacatttttaggtataacagacacatacacttagatcaatgcatcgctgtactccttgaaatcagagctagcttgttagaaaaagaatcagagccaggtatagcggtgcatgtcattaatcccagcgctaaagagacagagacaggagaatctctgtgagttcaaggccacatagaaagagcctgtctcgggagcaggaaaaaaaaaaaaaacggaattc

SEQ ID NO: 23 - protein, 399 aa, Mus musculus
MAMRPGPLWLLGLALCALGGGHGPRPPHTCPQRRLGARERRDMQREILAVLGLPGRPRPRAQPAAARQPASAPLFMLDLYHAMTDDDDGGPPQAHLGRADLVMSFVNMVERDRTLGYQEPHWKEFHFDLTQIPAGEAVTAAEFRIYKEPSTHPLNTTLHISMFEVVQEHSNRESDLFFLDLQTLRSGDEGWLVLDITAASDRWLLNHHKDLGLRLYVETADGHSMDPGLAGLLGRQAPRSRQPFMVTFFRASQSPVRAPRAARPLKRRQPKKTNELPHPNKLPGIFDDGHGSRGREVCRRHELYVSFRDLGWLDWVIAPQGYSAYYCEGECAFPLDSCMNATNHAILQSLVHLMKPDVVPKACCAPTKLSATSVLYYDSSNNVILRKHRNMVVKACGCH

SEQ ID NO: 24 - protein, 97 aa, Artificial Sequence
  Generic sequence of morphogenic polypeptides from several species with an optional cysteine at position 36
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 25 - protein, 102 aa, Artificial Sequence
  Generic sequence of morphogenic polypeptides from multiple species with optional cysteine at possition 41
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 26 - protein, 5 aa, Artificial Sequence
  N-terminal sequence of generic sequence 1 from morphogenic polypeptides from various species
CXXXX

SEQ ID NO: 27 - protein, 97 aa, Artificial Sequence
  Composite amino acid sequences of morphogenic proteins with six cysteine skeleton from various species
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 28 - protein, 102 aa, Artificial Sequence
  Composite amino acid sequences of morphogenic proteins with seven cysteine skeleton from multiple species
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 29 - protein, 101 aa, Artificial Sequence
  Generic amino acid sequence of morphogenic seven-cysteine skeleton from multiple species
CXXHELYVSFXDLGWXDWXIAPXGYXAYYCEGECXFPLXSXMNATNHAIXQXLVHXXXPXXVPKXCCAPTXLXAXSVLYXDXSXNVILXKKRNMVXACGCH

SEQ ID NO: 30 - protein, 4 aa, Artificial Sequence
  proteolytic site of morphogenic proteins from multiple species
RXXR

SEQ ID NO: 31 - protein, 4 aa, Homo sapiens
GGPP